(19) United States
(12) Patent Application Publication
(10) Pub. No.: US 2023/0022710 A1
(43) Pub. Date: Jan. 26, 2023

(54) SYSTEMS AND METHODS FOR PROCESSING AND ANALYZING KINEMATIC DATA FROM INTELLIGENT KINEMATIC DEVICES

(71) Applicant: CANARY MEDICAL SWITZERLAND AG, Baar (CH)

(72) Inventors: Patrick Aubin, Seattle, WA (US); Kin Chi Chan, Menlo Park, CA (US); Paul Detchemendy, San Diego, CA (US); Barbara Elashoff, San Mateo, CA (US); Kevin Gemmell, San Diego, CA (US); Jeffrey M. Gross, Carlsbad, CA (US); William L. Hunter, Vancouver (CA); Michael Kane, New Haven, CT (US); David Lee, New York, NY (US); Kimberly Salant, Asbury, NJ (US); John Savage, Fair Haven, NJ (US); Peter J. Schiller, San Marcos, CA (US)

(21) Appl. No.: 17/855,422

(22) Filed: Jun. 30, 2022

Related U.S. Application Data
(60) Provisional application No. 63/217,700, filed on Jul. 1, 2021.
(60) Provisional application No. 63/238,709, filed on Aug. 30, 2021.
(60) Provisional application No. 63/239,371, filed on Aug. 31, 2021.

(51) Int. Cl. G06F 30/20; A61B 5/00; A61B 5/11

(52) U.S. Cl. CPC G06F 30/20; A61B 5/4528; A61B 5/686; A61B 5/1114; A61B 2562/0219

(57) ABSTRACT

An apparatus for predicting an outcome of a patient includes a processor, and a memory storing computer executable instructions, which when executed by the processor cause the processor to perform operations comprising obtaining patient kinematic data of the patient; deriving one or more patient kinematic features from the patient kinematic data; and determining the outcome based on the one or more patient kinematic features and at least one additional data element of the patient using an outcome model trained on a training set of kinematic features of the same type as the patient kinematic features and the at least one additional data element.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### TECHNICAL FIELD

The present disclosure relates generally to systems and methods for processing and analyzing data from medical devices, and more particularly, to systems and method for processing and using kinematic data from intelligent kinematic devices to train kinematic classification models or other outcome models, to manage device configurations, and to monitor, assess, diagnose, and/or predict clinical outcomes (e.g., movement type, complications, adverse events, device condition, etc.).

### BACKGROUND

Using a knee implant as an example, current implantable medical devices for a total knee arthroplasty (TKA) typically consist of five components: a femoral component, a tibial component, a tibial insert, a tibial stem extension and a patella component. The patella component, which is implanted in front of the joint, is not shown in the figures. Collectively, these five components may be referred to as any one of an implantable medical device, a knee prosthetic system, or a total knee implant (TKI). Each of these five components may also be individually referred to as an implantable medical device. In either case, these components are designed to work together as a functional unit, to replace, provide, and/or enhance the function of a natural knee joint.

To this end, the femoral component is attached to the femoral head of the knee joint and forms the superior articular surface. The tibial insert (also called a spacer) is often composed of a polymer and forms the inferior articulating surface with the metallic femoral head. The tibial component consists of a tibial stem that inserts into the marrow cavity of the tibia and a base plate, which is sometimes called either a tibial plate, a tibial tray, or a tibial base plate that contacts/holds the tibial insert. Optionally, and particularly where the proximal tibial bone quality and/or bone quantity is compromised, a tibial stem extension can be added to the tibial stem of the tibial component, where the tibial stem extension serves as a keel to resist tilting of the tibial component and increase stability.

Commercial examples of TKA products include the Persona™ knee system (I113369) and associated tapered tibial stem extension (K133737), both by Zimmer Biomet Inc. (Warsaw, Ind., USA). The surgery whereby these four components are implanted into a patient is also referred to as a total knee arthroplasty (TKA). Similar prosthetic devices are available for other joints, such as total hip arthroplasty (THA) and shoulder arthroplasty (TSA), where one particular surface is metallic, and the opposing surface is polymeric. Collectively, these devices and procedures (TKA, THA and TSA) are often referred to as total joint arthroplasty (TJA) or partial joint arthroplasty (PJA) if only one joint surface is replaced.

For a TKA, the tibial component and the femoral component are typically inserted into, and cemented in place within, the tibia bone and femoral bone, respectively. In some cases, the components are not cemented in place, as in uncemented knees. Regardless of whether they are cemented in place or not, once placed and integrated into the surrounding bone (a process called osseointegration), they are not easy to remove. Accordingly, proper placement of these components during implantation is very important to the successful outcome of the procedure, and surgeons take great care in implanting and securing these components accurately.

Current commercial TKA systems have a long history of clinical use with implant duration regularly exceeding 10 years and with some reports supporting an 87% survivorship at 25 years. Clinicians currently monitor the progress of TKA patients post implant using a series of in-office appointments including physical examinations at 2-4 weeks, 6-8 weeks, 3 months, 6 months, 12 months post-operatively, and yearly thereafter.

After the TKI has been implanted, and the patient begins to walk with the knee prosthesis, problems may arise and are sometimes hard to identify. Clinical exams are often limited in their ability to detect failure of the prosthesis; therefore, additional monitoring is often required such as CT scans, MRI scans or even nuclear scans. Given the continuum of care requirements over the lifetime of the implant, patients are encouraged to visit their clinician annually to review their health condition, monitor other joints, and assess the TKA implant's function. While the current standard of care affords the clinician and the healthcare system the ability to assess a patient's TKA function during the 90-day episode of care, the measurements are often subjective and lack temporal resolution to delineate small changes in functionality that could be a pre-cursor to larger mobility issues. The long-term (>1 year) follow up of TKA patients also poses a problem in that patients do not consistently see their clinicians annually. Rather, they often seek additional consultation only when there is pain or other symptoms.

Currently, there is no mechanism for reliably detecting misplacement, instability, or misalignment in the TKA without clinical visits and the hands and visual observations of an experienced health care provider. Even then, early identification of subclinical problems or conditions is either difficult or impossible since they are often too subtle to be detected on physical exam or demonstratable by radiographic studies. As a result, it is often difficult to detect complications early in their evolution when non-surgical correction might still be possible. Late detection of many common complications can necessitate manipulation under anesthesia (MUA) and/or replacement of all or part of the prosthesis, making early diagnosis particularly valuable. Furthermore, if early detection were possible, corrective actions would be hampered by the fact that the specific amount of movement and/or degree of improper alignment cannot be accurately measured or quantified, making targeted, successful intervention unlikely. Existing external monitoring devices do not provide the fidelity required to detect instability since these devices are separated from the TKA by skin, muscle, and fat—each of which masks the mechanical signatures of instability and introduce anomalies such as flexure, tissue-borne acoustic noise, inconsistent sensor placement on the surface, and inconsistent location of the external sensor relative to the TKA.

Implants other than TKA implants may also be associated with various complications, both during implantation and post-surgery. In general, correct placement of a medical implant can be challenging to the surgeon and various complications may arise during insertion of any medical implant (whether it is an open surgical procedure or a minimally invasive procedure). For example, a surgeon may wish to confirm correct anatomical alignment and placement of the implant within surrounding tissues and structures. This can, however, be difficult to do during the procedure itself, making intraoperative corrective adjustments difficult.

In addition, a patient may experience a number of complications post-procedure. Such complications include neurological symptoms, pain, stiffness in extension and/or contraction, malfunction (blockage, narrowing, loosening, etc.) and/or wear of the implant, movement or breakage of the implant, bending or deformation of the implant, inflammation and/or infection. While some of these problems can be addressed with pharmaceutical products and/or further surgery, they are difficult to predict and prevent; often early identification of complications and side effects, although desirable, is difficult or impossible.

It is an object of the present invention to overcome the problems known from the prior art.

### SUMMARY

Briefly stated, the present disclosure relates to an intelligent implant that includes an implantable medical device and an implantable reporting processor (IRP) that is associated with the implantable medical device and is configured for placement in boney tissue surrounded by muscle.

Systems and methods process and analyze kinematic data from intelligent kinematic devices to train kinematic classification models or other outcome models, to manage device configurations, and to monitor, assess, diagnose, and/or predict clinical outcomes (e.g., movement type, complications, adverse events, device condition, etc.).

In some aspects, the techniques described herein relate to a computer-implemented method for generating a patient movement classification model, wherein the computer-implemented method includes, as implemented by a computing system including one or more computer processors: obtaining a plurality of records from across a patient population, wherein a record of the plurality of records includes kinematic data representing motion of an implant implanted in a patient of the patient population, and wherein the implant includes a plurality of sensors configured to detect motion of the implant; for individual records of the plurality of records: identifying one or more elements represented by the kinematic data; determining one or more kinematic features based on the one or more elements; and labeling the one or more kinematic features with a movement type of a plurality of movement types to generate one or more labeled kinematic features, wherein each movement type of the plurality of movement types is associated with movement of a body part; and training a machine learning model using the labeled kinematic features to classify motion of a particular implant as a particular movement type.

In some aspects, the techniques described herein relate to a system including: an implant configured to be implanted into a patient, wherein the implant includes a plurality of sensors configured to detect motion of the implant; one or more computer processors programmed by executable instructions to at least: receive a plurality of records from the implant, wherein a record of the plurality of records includes kinematic data representing motion of the implant; determine one or more kinematic features based on the kinematic data; determine, based at least partly on the one or more kinematic features, a movement type of a plurality of movement types, wherein the movement type is associated with movement of a body part of the patient.

This Summary has been provided to introduce certain concepts in a simplified form that are further described in detail below in the Detailed Description. Except where otherwise expressly stated, this Summary is not intended to identify key or essential features of the claimed subject matter, nor is it intended to limit the scope of the claimed subject matter.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIGS. 1A, 1B, and 1C are illustrations different total joint arthroplasty systems with intelligent implants including a total knee arthroplasty system (FIG. 1A), a total hip arthroplasty system (FIG. 1B), and total shoulder arthroplasty system (FIG. 1C).

FIG. 2A is an illustration of an intelligent implant in the form of a tibial component of a knee prosthesis implanted in a tibia and including an implantable reporting processor.

FIG. 2B is an illustration of an implantable reporting processor.

FIG. 3 is an exploded view of the tibial component of FIG. 2A.

FIG. 4 is a side view the implantable reporting processor of FIG. 2A.

FIG. 5 is a block diagram of an implantable reporting processor (IRP).

FIG. 6 is a perspective view of the IRP of FIG. 4 implanted in a tibia of a knee, and showing a set of coordinate axes within the frame of reference of the IRP.

FIG. 7 is a front view of a standing patient in which the IRP of FIG. 6 is implanted and of two of the coordinate axes of the IRP.

FIG. 8 is a side view of the patient of FIG. 7 in a supine position and of two of the coordinate axes of the IRP.

FIG. 9A is a plot, versus time, of acceleration signals aₓ(g), a_{y}(g), and a_{z}(g) (in units of g-force) generated in response to accelerations along the x axis, the y axis, and the z axis of FIG. 6 while the patient of FIG. 7 is walking forward with a normal gait at speeds of 0.5 meters/second.

FIG. 9B is a plot, versus time, of angular-velocity signals Ωₓ(dps), Ω_{y}(dps), and Ω_{z}(dps) (in units of degrees per second) generated in response to angular velocities about the x axis, the y axis, and the z axis of FIG. 6 while the patient is walking forward with a normal gait at a speed of 0.5 meters/second.

FIG. 10A is a plot, versus time, of acceleration signals aₓ(g), a_{y}(g), and a_{z}(g) (in units of g-force) generate in response to accelerations along the x axis, the y axis, and the z axis of FIG. 6 while the patient of FIG. 7 is walking forward with a normal gait at speeds of 0.9 meters/second.

FIG. 10B is a plot, versus time, of angular-velocity signals Ωₓ(dps), Ω_{y}(dps), and Ω_{z}(dps) (in units of degrees per second) generated in response to angular velocities about the x axis, the y axis, and the z axis of FIG. 6 while the patient is walking forward with a normal gait at a speed of 0.9 meters/second.

FIG. 11A is a plot, versus time, of acceleration signals aₓ(g), a_{y}(g), and a_{z}(g) (in units of g-force) generate in response to accelerations along the x axis, the y axis, and the z axis of FIG. 6 while the patient of FIG. 7 is walking forward with a normal gait at speeds of 1.4 meters/second.

FIG. 11B is a plot, versus time, of angular-velocity signals Ωₓ(dps), Ω_{y}(dps), and Ω_{z}(dps) (in units of degrees per second) generated in response to angular velocities about the x axis, the y axis, and the z axis of FIG. 6 while the patient is walking forward with a normal gait at a speed of 1.4 meters/second.

FIG. 12A is a block diagram showing how implant parameters and raw acceleration and gyroscopic data are retrieved from a database and processed into gait parameters

FIG. 12B is an illustration of an implant coordinate system.

FIG. 12C is an illustration of a tibia coordinate system relative to an implant coordinate system.

FIG. 12D is a graph showing how qualified gait cycles are identified by the gait cycle parser.

FIG. 12E is a block diagram showing how “qualified gait cycles” get parsed from raw acceleration and gyroscopic data given a set of qualification requirements.

FIG. 12F is an illustration of an implant relative to a tibia length.

FIG. 12G are top view illustrations of different alignments of an implant relative to a patient's tibia.

FIG. 12H is a trigonometric diagram showing how the transverse plane skew angle is calculated from the first principal component (P1) of the angular velocity matrix (W).

FIG. 12I is an illustration of a tibia coordinate system (tib) and relative to a ground (gnd) coordinate system when walking.

FIG. 12J is an illustration of angular velocity of the tibia in the sagittal plane.

FIG. 12K is a graph of tibia sagittal plane angle with respect to ground as a function of sample number.

FIG. 13 is a schematic diagram of motion of a leg.

FIG. 14 is a flow chart of a method of data sampling that is implemented by the implanted reporting processor of FIG. 5.

FIG. 15 is a block diagram of a system that obtains and processes kinematic data from kinematic implantable devices and uses the data to train machine-learned classification models, to classify motion activity associated with intelligent implants as different types of movements, to track patient recovery and/or implant conditions, and to configure implants to sense motion activity.

FIGS. 16A, 16B, 16C, and 16D are functional block diagrams of a training apparatus of FIG. 15 for generating machine-learned movement classification models based on records of motion activity.

FIG. 17 is an illustration of a raw kinematic signal representation of raw kinematic data obtained from a sensor associated with the tibia and representing motion activity corresponding to a normal gait cycle.

FIG. 18A is an illustration of a filtered version of the raw kinematic signal of FIG. 17.

FIG. 18B is an illustration of the kinematic signal of FIG. 18A marked to indicate different elements in the signal, each element corresponding to a fiducial point C, H, I, R, P, and S of the signal.

FIG. 18C is an illustration of different phases and different events of a normal gait cycle together with fiducial points C, H, I, R, P, and S of the kinematic signal of FIG. 18B.

FIG. 18D is an illustration of the kinematic signal of FIG. 18B marked to indicate different kinematic features that may be derived based on the fiducial points C, H, I, R, P, and S of the signal.

FIG. 19A is an illustration of a kinematic signal sensed during normal walking by a patient relative to a kinematic signal sensed during limping with pain by the patient, together with example kinematic features calculated by the apparatus of FIGS. 16A-16D.

FIG. 19B is an illustration of a kinematic signal sensed during normal walking by another patient relative to a kinematic signal sensed during limping with pain by the patient, together with example kinematic features calculated by the apparatus of FIGS. 16A-16D.

FIG. 19C is an illustration of a kinematic signal sensed during normal walking by a patient relative to a kinematic signal sensed during walking with a limited range of motion by the patient, together with example kinematic features calculated by the apparatus of FIGS. 16A-16D.

FIG. 20 is a functional block diagram of a classification apparatus of FIG. 15 that includes a machine-learned movement classification model generated by the training apparatus of FIGS. 16A-16D that identifies movement types based on records of motion activity.

FIG. 21 is a functional block diagram of a benchmark apparatus for generating a recovery benchmark module that provides benchmark information for tracking the recovery of a subject patient relative to a similar patient population or tracking the condition of a surgical implant.

FIGS. 22A, 22B, and 22C are example recovery tracker curves illustrating different parameters of recovery for a patient relative to percentile curves across a patient population, including range of motion (FIG. 22A), walking speed (FIG. 22B), and cadence (FIG. 22C).

FIG. 23 is a functional block diagram of a tracking apparatus of FIG. 15 for tracking patient recovery and/or implant condition relative to a similar patient population.

FIG. 24 is a functional block diagram of a configuration management apparatus of FIG. 15 for managing operational parameters of the kinematic implantable devices of FIG. 15 to improve the collection of data.

FIG. 25 is a schematic diagram of the training apparatus of FIG. 16.

FIG. 26 is a schematic diagram of the classification apparatus of FIG. 20.

FIG. 27 is a schematic diagram of the benchmark apparatus of FIG. 21.

FIG. 28 is a schematic diagram of the tracking apparatus of FIG. 23.

FIG. 29 is a schematic diagram of the configuration management apparatus of FIG. 24.

FIG. 30 are illustrations of a kinematic signal sensed across all channels of a six-channel IMU associated with a tibia, during normal walking by a patient.

FIG. 31 are illustrations of raw kinematic signals sensed across all channels of a six-channel IMU associated with a tibia, while a patient is walking with knee pain.

FIG. 32 are illustrations of raw kinematic signals sensed across all channels of a six-channel IMU associated with a tibia, while a patient is walking with contracture (limited range of motion).

FIG. 33 are illustrations of a kinematic signal sensed across three accelerometer channels of an IMU associated with a hip, during normal walking by a patient.

FIG. 34 are illustrations of a kinematic signal sensed across three gyroscopes channels of an IMU associated with a hip, during normal walking by a patient.

FIG. 35A are illustrations of different clusters of similar kinematic signals.

FIG. 35B are illustrations of different kinematic signals that are assigned different labels.

FIG. 36A is an illustration of a spectral distribution graph derived from a kinematic signal sensed by an IMU associated with a tibia.

FIG. 36B is an illustration of a spectral distribution graphs derived from a kinematic signal sensed by a gyroscope of an IMU associated with a tibia, during normal walking by a patient.

FIG. 36C is an illustration of a spectral distribution graphs derived from a kinematic signal sensed by a gyroscope of an IMU associated with a tibia, during limping by a patient.

FIG. 37 are illustrations of a raw kinematic signal sensed across three gyroscope channels of an IMU associated with a shoulder, during normal movement by a patient.

FIG. 38 are illustrations of a raw kinematic signal sensed across three accelerometer channels of an IMU associated with a shoulder, during normal movement by a patient.

FIG. 39A is an illustration of a user interface display showing a gait classification of abnormal walking based on a set of kinematic features including swing velocity, reach velocity, knee range of motion, and stride length.

FIG. 39B is an illustration of a user interface display showing a gait classification of normal walking based on a set of kinematic features including swing velocity, reach velocity, knee range of motion, and stride length.

FIG. 40 is a 3D rendering of an exemplary wearable device of the present disclosure. The wearable device of FIG. 40 includes a casing or housing, within which electronic components are held. The housing includes features that allow the wearable device to be secured to a subject, where in FIG. 40 those features are two holes through which a strap may pass (only one of the two holes is shown in the drawing) and then that strap also goes around the leg of the subject. In the drawing, an extruding portion of the housing is present, inside of which an antenna may be located.

FIG. 41 is a line drawing of the exemplary wearable device of FIG. 40, which shows both openings through which a flexible strap may pass to secure the device to a subject. The drawing of FIG. 41 also shows a concave region which is contoured to fit snugly around a portion of the tibia (shin bone) of the subject.

FIG. 42 is a line drawing of the wearable device of FIG. 41, from the perspective of the top of the device, in particular showing the concave portion which fits around a portion of a tibia of a subject.

FIG. 43 is a drawing that shows exemplary internal electronic components for a wearable device of the present disclosure, some (i.e., one or more) or all of which may be present in a wearable device of the present disclosure, and how those components may be positioned relative to the skin of the subject (patient). The housing is denoted as the plastic enclosure in this drawing.

FIG. 44 shows an optional placement of an exemplary wearable device of the present disclosure when the device is secured to a subject. Only selected bones of the subject are shown in the drawing. In the drawing, the wearable device is secured near the top of the tibia bone. The tuberosity of the tibia or tibial tuberosity or tibial tubercle is an elevation on the proximal, anterior aspect of the tibia, just below where the anterior surfaces of the lateral and medial tibial condyles end.

FIG. 45 shows a top view of a charger of the present disclosure which may be used to provide power to a wearable device of the present disclosure. The charger of the present disclosure may have a shape that mates with the shape of the wearable device, such as the device of FIGS. 40, 41, 42 and 43, where this shape is present in the cradle portion of the charger. The charger also has a cable, optionally referred to as a power cord, that transmits power from a power source (e.g., an electrical outlet or a USB port) to the charger, and from the charger to a wearable device of the present disclosure.

FIG. 46 shows a side view of the charger of FIG. 45.

FIG. 47 shows a perspective view of a charger of the present disclosure as also shown in top view in FIG. 45, which may be used to provide power to a wearable device of the present disclosure. The charger of the present disclosure has a shape that mates with the shape of a wearable device of the present disclosure, such as the device of FIGS. 40, 41, 42 and 43.

FIG. 48 shows the mating of the cradle of the charger of FIGS. 45, 46 and 47 with the wearable device of FIGS. 40, 41, 42 and 43, where such mating is advantageous to create proper alignment between the charger and the wearable device to achieve effective charging of the wearable device by the charger. Thus, in one embodiment the present disclosure provides a system comprising a wearable device of the present disclosure and a charger for the wearable device. The charger provides power to the wearable device, thereby replacing power that is consumed by the wearable device during its operation. In one embodiment the charger includes a cradle and a power cord (also referred to as a cable or a power cable), where the cradle is contoured to conform to a shape of the wearable device, so that the cradle mates to a portion of the wearable device and holds the wearable device in a secure position during charging.

### DETAILED DESCRIPTION

The present disclosure may be understood more readily by reference to the following detailed description of preferred embodiments of the disclosure and the examples of implantable medical devices with implantable reporting processors included herein. The following description, along with the accompanying drawings, sets forth certain specific details in order to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components that are associated with the environment of the present disclosure, including but not limited to the communication systems and networks, have not been shown or described in order to avoid unnecessarily obscuring descriptions of the embodiments.

Disclosed herein are systems and methods for obtaining, processing and analyzing kinematic data obtained from an implantable or externally worn device. An implantable device may be referred to herein as an intelligent implant. The system and method collect relevant data on patients as they recover from surgical procedures and motivates new approaches and interventions for both increasing the likelihood of a successful recovery and early identification of complications as well as providing opportunities for longer-term aspects of health related to the procedure and beyond. Advantageously, systems and methods described herein may evaluate kinematic data obtained from a single device per patient, such as a single intelligent implant or externally worn device (e.g., on or adjacent to one body part associated with a joint, such as a tibia). This is in contrast to studies that may make use of multiple devices per patient to generate data for evaluation (e.g., a device above a knee or other joint, such as on or adjacent to a femur, and another device below the joint, such as on or adjacent to a tibia). In one aspect the present disclosure is directed to identifying, locating and/or quantifying problems associated with medical implants, particularly at an early stage, and providing methods and devices to remedy these problems.

Generally, and unless otherwise specifically stated, any system or method described herein for obtaining, processing and analyzing data obtained from an intelligent implant may be applied to data obtained from an externally worn device, and vice versa.

In one embodiment, the intelligent implant is a knee arthroplasty device for patients undergoing knee replacement and includes an inertial measurement unit (IMU). These devices are able to capture orientation and movement information of the device (and the knee in which it is implanted) and upload those data periodically to a central location where it can be processed and analyzed. Connecting the IMU data to these health opportunities can be facilitated by the careful construction of clinically relevant biomarkers that can capture diagnostic, prognostic and potentially predictive features which can then be used to understand and characterize patient populations as well as evaluate the individual-level recovery process. Biomarker development begins by understanding the data, which are collected over short periods called “bouts.” Each bout is represented by multi-channel data, such as data from six separate channels capturing acceleration as well as rotation on each of three axes sampled. Bouts may be recorded based on user input, time of day, or may be triggered based on movement.

Disclosed herein is an IMU tools package, which in one embodiment encapsulates preprocessing functionality as well as providing tools facilitating the creation of biomarkers. The package provides signal processing utilities for analyzing frequency-domain characteristics of bouts, filtering bouts, and visualizing both their spatial and frequency characteristics. Based on this signal processing, the systems and methods detect walking activity, partition walking activity into steps, extract clinically relevant features of a step, and how those step features can be used to evaluate patient prognosis including pain, mobility, and stiffness.

The present disclosure refers to TJA (total joint arthroplasty) which term includes reference to the surgery and associated implantable medical devices such as a TJA prosthesis. Features of methods, devices and systems of the present disclosure may be illustrated herein by reference to a specific TJA prosthesis, however, the disclosure should be understood to apply to any one or more TJA prosthesis, including a TKA (total knee arthroscopy) prosthesis, such as a TKI (total knee implant) which may also be referred to as a TKA system; a PKA (partial knee arthroplasty) system; a TSA (total shoulder arthroscopy) prosthesis, such as a TSI (total shoulder implant) which may also be referred to as a TSI system; a PSA (partial shoulder arthroplasty) system; a THA (total hip arthroscopy) prosthesis, such as a THI (total hip implant) which may also be referred to as a THA system; a PHA (partial hip arthroplasty) system; and other joint replacement systems for elbows, ankles and intervertebral discs.

An “implantable medical device” as used in the present disclosure, is an implantable or implanted medical device that desirably replaces or functionally supplements a subject's natural body part. As used herein, the term “intelligent implant” refers to an implantable medical device with an implantable reporting processor, and is interchangeably referred to as a “smart device.” When the intelligent implant makes kinematic measurements, it may be referred to as a “kinematic implantable device.” In describing embodiments of the present disclosure, reference may be made to a kinematic implantable device, however it should be understood that this is exemplary only of the intelligent medical devices which may be employed in the devices, methods, systems etc. of the present disclosure. Another example of an intelligent medical device is a wearable device. As the context allows, reference herein to methods of processing data from an intelligent implant or an implantable medical device should be understood to also be applicable to the processing of data from a wearable device of the present disclosure.

A “wearable device” or a “wearable medical device” as used in the present disclosure refers to a wearable device that is configured for being secured to a joint or a limb of a mammal, e.g., a person, referred to herein as a subject or the subject. Securing the wearable device includes holding the device at the intended location on the subject, e.g., holding the device secured to a location on the leg or shoulder. Securing the device also includes holding the device in a constant, or near constant configuration relative to the body part of the subject to which the device is secured. In one embodiment, a secured device maintains its positioning at the intended location on the subject and also maintains its orientation. For example, the device does not rotate either clockwise or counterclockwise after being secured to the body part, which movement would be an example of an undesirable change in configuration of the device after it has been secured to a body part of the subject. To secure the configuration of the wearable device, the housing of the device may have a shape that is complementary to the shape of the location where the device should be secured. For example, the housing may include a “V” shape which is contoured to fit around the shin of the subject.

References herein to a “device” may generally be interpreted to apply to either an implantable medical device or a wearable medical device.

The device contains one or more sensors as discussed herein that can detect changes in the environment of the device. For example, the device may contain a kinematic sensor that detects movement of the device and accordingly measures movement of the part of the subject to which the device is secured. Measurement of movement may include, for example, one or more of extent of movement, direction of movement, rate of movement and frequency of movement. When movement is walking, the measurement may provide data to determine gait parameters, such as cadence, stride length, walking speed, tibia range of motion, knee range of motion, step count and distance traveled. The step count, distance traveled, and cadence represent measures of activity and robustness of activity. In one embodiment, the wearable device is a wearable medical device having clinical application.

Examples of a kinematic sensor include accelerometer and gyroscope. In one embodiment, the device includes an accelerometer. In one embodiment, the device includes a gyroscope. Optionally, the gyroscope and accelerometer capture data samples between 25 Hz and 1,600 Hz. In one embodiment, the device includes a magnetometer, where a magnetometer provides orientation information of the device's location with respect to Earth (allows for true orientation).

The device optionally contains a memory to store the information obtained by the sensor. The device optionally includes a second memory to store firmware that provides operating instructions to the device.

The device contains a power source to provide power to the sensor and other features of the device that require power. In one embodiment the power source is a battery, optionally a rechargeable battery, or optionally a non-rechargeable battery. Optionally, the device includes an indicator of the amount of power available in the power source, optionally as a percentage of total possible power available in the power source. The power supply of the device may be recharged as needed, optionally in view of the indicator of the amount of power available in the power supply.

The device optionally contains telemetric capability. Telemetric capability allows the device to transmit the information obtained by the sensor to another device, e.g., a computer or a network or the cloud. Telemetric capability may also allow the device to receive and respond to electronic signals, such as instructions to make a measurement, or instructions to transmit stored information to outside the device. An antenna may be present as part of the device to facilitate telemetric capability. The telemetry capability of a wearable device may be compatible with, or identical to the telemetry capability of an implanted medical device. For example, the telemetric capability may provide for Bluetooth capabilities.

The device may optionally be able to process data collected from sensors into clinically relevant metrics/parameters. Optionally, all or a portion of the data collected from the sensors is transmitted via telemetry to a location outside of the device, whereupon that collected data is processed into clinically relevant metrics/parameters.

In one embodiment the wearable device is hermetically sealed so that no fluid may flow between the exterior of the device and the sensor of the device. In one embodiment the wearable device is not hermetically sealed, however has ingress protection in that a barrier is provided to fluid flow between the exterior of the device and the sensor.

In one embodiment, the wearable device is configured for being secured to a location on the subject near where the subject has, or intends to have, an implanted prosthesis. For example, when the subject has received, or intends to receive, either a full or partial knee replacement, the wearable device may be configured for being secured to either above or below the knee, depending on the details of the prosthesis. In one embodiment, the subject has, or intends to have a total knee arthroplasty (TKA) or a partial knee arthroplasty (PKA). As another example, when the subject has received, or intends to receive, a hip replacement, the wearable device may be configured for being secured on or near the hip, e.g., around the upper leg of the subject. Yet another example is when the subject has received, or intends to receive, a shoulder prosthesis, in which case the wearable device may be configured for being secured on or near the shoulder, e.g., around the upper arm of the subject.

Thus, in one embodiment the present disclosure provides a wearable device that is configured for being secured to a joint or a limb of a subject, and more specifically to a location where the subject has, or intends to have, an implanted prosthesis. The device of the embodiment includes a sensor, a power supply, a memory, and telemetric capability. The implanted prosthesis may optionally include a sensor, a power supply, a memory, and telemetric capability. When each of the wearable device and the implanted prosthesis includes a plurality of sensors, those sensors may be arranged in a similar or identical configuration. For example, the sensors may be secured to a circuit board, and the same circuit board is present in both the wearable device and the implanted prosthetic device, where the x, y, and z directions of the circuit board are the same in both devices. Stated another way, two or more sensors present in the wearable device may be aligned with equivalent sensors present in the implanted prosthetic device. In this way, sensor data obtained from the wearable device is analogous to and may be correlated with sensor data obtained from the implanted prosthetic device. Those sensors may be selected from, for example, accelerometers and gyroscopes, where optionally the accelerometer and gyroscope capture data samples between 25 Hz and 1,600 Hz.

As mentioned previously, the wearable device may include a magnetometer that informs orientation of the device's location. This orientation information may be used to assist in correlating data obtained from the wearable device with data obtained from an implanted device or even with data obtained from a second wearable device.

In one embodiment, the wearable device of the present disclosure is configured to be secured below the knee of the subject and provides information that characterizes the gait of the subject wearing the wearable device. The gait information may be obtained from a single worn device of the present disclosure rather than, e.g., two externally affixed devices that are placed one above the knee and the other below the knee of the subject. A single device is advantageous compared to two devices in terms of cost and convenience. With a single wearable device of the present disclosure, gait information including, for example, range of motion of the knee during walking (functioning) and the presence or absence of limping while walking, and the degree of limping if present, may be determined.

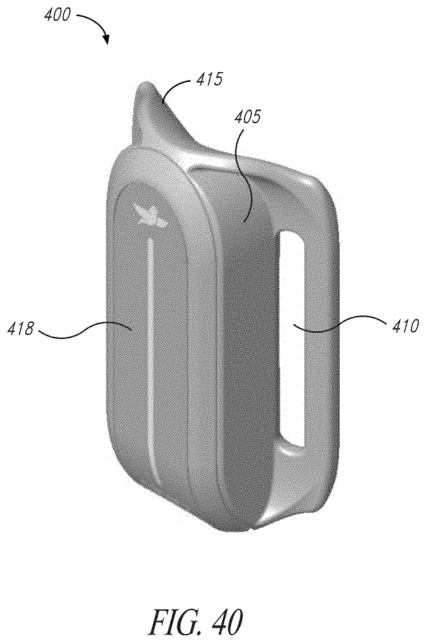

FIG. 40 is a 3D rendering of an exemplary wearable device of the present disclosure. The wearable device (400) of FIG. 40 includes a casing or housing (405), within which electronic components are held. The housing includes features that allow the wearable device to be secured to a subject, where in FIG. 40 those features are two holes (410) through which a strap may pass (only one of the two holes is shown in the drawing) and then that strap also goes around the leg of the subject. Instead of holes and an associated strap, the device may be secured to the subject by other means, for example, by self-adhesive tape. In the drawing, an extruding portion of the housing (415) is present, inside of which an antenna may be located. The extruding portion (415) is an optional portion of the housing (405), where a housing (405) that lacks an extruding portion (415) may have the antenna positioned within the housing at a non-extruding location of the housing. Analogously, straps or other securing features, such as self-adhesive tape, may be used to secure the device (400) to anatomy of the subject, e.g., to a shoulder or to a hip of a subject.

A portion of the housing (405) may be configured to function as a power receiving surface (418), where the power receiving surface (418) may be utilized as an area through which power may be transmitted into the device from a charging device, in order to recharge a battery inside the device (400).

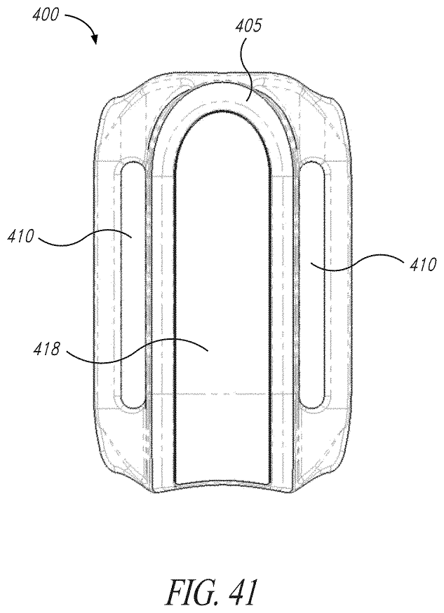

FIG. 41 is a line drawing of the exemplary wearable device of FIG. 40, which shows both openings (410) through which a flexible strap may pass to secure the device to a subject. The drawing of FIG. 41 also shows the power receiving surface (418).

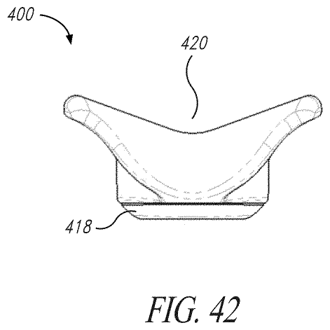

FIG. 42 is a line drawing of the wearable device (400) of FIG. 41, from the perspective of the top of the device, in particular showing the power receiving surface (418) and the contoured portion (420) which fits around a portion of a tibia of a subject. The power receiving surface (418) may be said to be located on the front or face of the device while the portion (420) which fits against the subject wearing the device, may be said to be located on the back or rear of the device. That contour may be adjusted to fit snugly against a different part, e.g., a different limb or part of a limb, of the subject's anatomy if the device is not placed around a portion of the tibia, but instead is placed against, e.g., a shoulder or associated arm, or a hip or associated leg. In one embodiment the contoured surface (420) is designed to be secured to a portion, e.g., a limb, of the subject wearing the device.

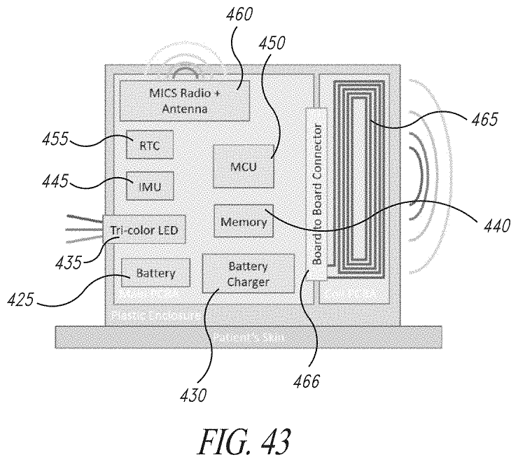

FIG. 43 is a drawing that shows exemplary internal electronic components for a wearable device of the present disclosure, some (i.e., one or more) or all of which may be present in a wearable device of the present disclosure, and how those components may be positioned relative to one another and relative to the skin of the subject (patient). The housing is denoted as the plastic enclosure in this drawing.

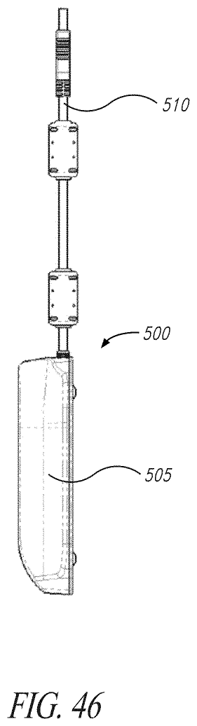

In FIG. 43, exemplary electronic components which may be present in a wearable device of the present disclosure, e.g., wearable device (400) of FIG. 40, FIG. 41 and FIG. 42 are shown. Those components include a battery which serves as a power source (425) for the device; a battery charger connection (430) which may be connected to a charger (not shown in FIG. 43) in order to recharge the battery (425), e.g., the charger shown in FIG. 46; an LED such as a tri-color LED (435) which is indicative of the status of the device, where the LED may change color depending on, for example, the level of power in the battery to thereby indicate when battery charging should be performed, when the device is or is not in wireless communication with a base station, when data is or is not being collected, if there is a fault in the device, etc.; a memory (440) which may be configured to, e.g., store data obtained from one or more sensors and/or to store information that facilitates logging of the device (such as an internal electronic self-test fail); an inertial measurement unit (IMU) (445) configured to capture orientation and movement information of the device (for the limb to which it is secured) and provide generated data to the memory (440); a microcontroller (MCU) integrated circuit (450), a Real-Time Clock (RTC) integrated circuit (455), a telemetry circuit including an antenna (460) to transmit data from the memory to a location outside of the device. As shown in FIG. 43, feature (465) is a wireless charging coil PCBA which allows for wireless charging. The coil PCBA (465) is oriented and facing the flat surface (418) and is as close to the outer surface of the device as possible to allow for most efficient wireless charging.

In one embodiment of the device (400) there are two printed circuit board assemblies (PCBA's). One PCBA may be referred to as the Main PCBA which contains electronic components 425, 430, 435, 440, 445, 450, 455, and 460 referred to above. The other PCBA may be referred to as the wireless charging Coil PCBA (465) also referred to above. The device (400) may also include a board to board Connector (466) located between the Main PCBA and Coil PCBA which allows the wireless charge from the unshown charger to then be connected to the Main PCBA such that the battery is recharged.

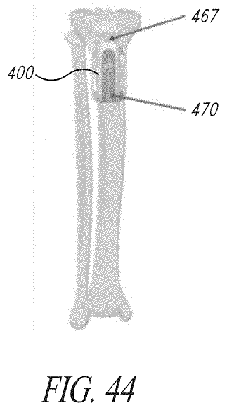

FIG. 44 shows an optional placement of an exemplary wearable device of the present disclosure when the device is secured to a subject. Only selected bones of the subject are shown in the drawing. In the drawing, the wearable device (400) is secured near the top of the tibia bone. The tuberosity of the tibia (467) or tibial tuberosity or tibial tubercle is an elevation on the proximal, anterior aspect of the tibia, just below where the anterior surfaces of the lateral and medial tibial condyles end. When the wearable device is secured to a different limb, it may be configured to be secured very close to an implantable medical device that is placed within the bone of that limb, e.g., a humerus or a femur, during a joint arthroplasty, where the implantable medical device may have sensors such as an accelerometer and/or gyroscope. The device (400) is optionally placed in this particular location shown in FIG. 44 so that it is very close to an implantable medical device that may be placed in the tibia of a subject, and which will also have sensors etc. to monitor movement of the subject.

In one embodiment, the external device of the present disclosure, e.g., the device (400) has a portion of the surface of its housing that is shaped in a complementary manner to the tibial tubercle, so that the device may be secured to the subject and held in place in against the tibial tubercle on the skin or clothing of the subject adjacent to the tibial tubercle. In one embodiment, the external device of the present disclosure, e.g., the device (400), has a portion of the surface of its housing that is shaped in a complementary manner to the tibia, so that the device may be secured to the subject and held in place against the tibia, on the skin or clothing of the subject adjacent to the tibia (shinbone), e.g., just below (towards the foot) of the tibial tubercle as shown in FIG. 44.

The external device of the present disclosure may comprise a mark, visible to the subject wearing the device, which informs the subject as to the direction that the wearable device should be located vis-h-vis the underlying body part. In FIG. 44, that mark (470) is a straight line which runs in the same direction as the tibia (i.e., from the knee to the ankle, i.e., from the lateral condyle of the tibia to the medial malleolus of the tibia). This mark may be referred to as an alignment mark (470).

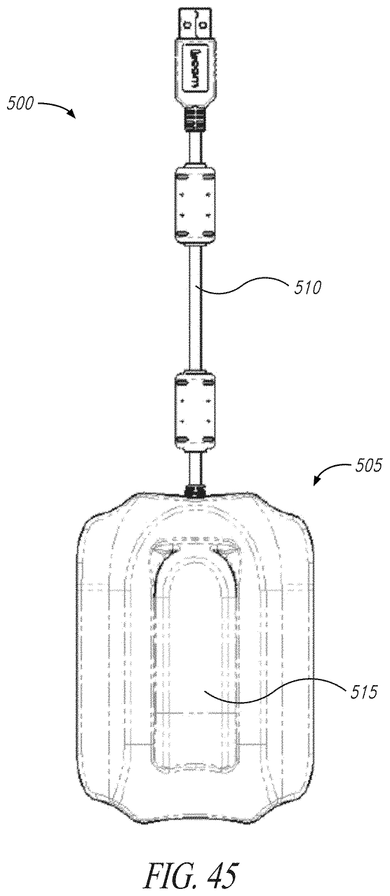

FIG. 45 shows a top view of a charger (500) of the present disclosure which may be used to provide power to a wearable device of the present disclosure. The charger (500) includes a cradle (505) and a cable (510). A portion of the outer surface of the charger of the present disclosure, and in particular a portion of the outer surface of the cradle (505), may be referred to a power providing surface (515) and may have a shape, e.g., a cavity having a shape or contour, that mates with a portion of the outer surface of a wearable device of the present disclosure, and in particular a power receiving surface (e.g., feature 418 in FIG. 40) such as the device of FIGS. 40-45, where this shape is present as a cavity in the cradle portion (505) of the charger (500). The charger also has a cable (510), optionally referred to as a power cord, that transmits power from a power source (e.g., an electrical outlet or a USB port) to the charger, and from the charger to a wearable device of the present disclosure. The charging portion (515) of the charger may have a contoured surface that is shaped to mate with the shape of a wearable device of the present disclosure, e.g., the device of FIGS. 40-45. Optionally, the charger could be flat (no cavity or contoured cradle) and the wearable would rest on the flat surface

FIG. 46 shows a side view of the charger of FIG. 45. In FIG. 46, the charger (500) includes a cradle (505) and a cable (510). In FIG. 46 the power providing surface (515 of FIG. 45) is not visible because that surface (515) is a concave surface in that it extends inwards toward the center of the cradle.

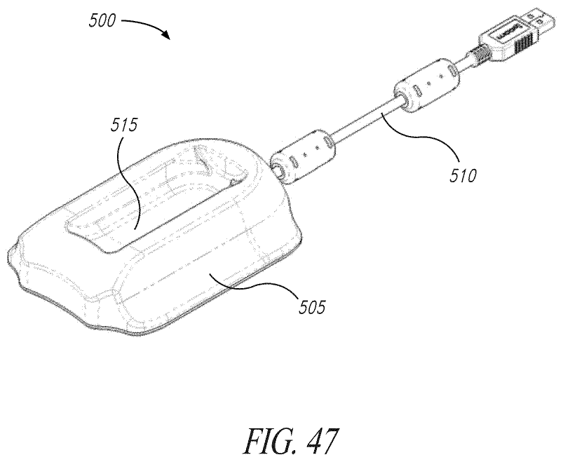

FIG. 47 shows an isometric three-dimensional view of a charger (500) of the present disclosure as also shown in top view in FIG. 45 and inside view in FIG. 46, which may be used to provide power to a wearable device of the present disclosure. The charger (500) includes a cable (510) and a cradle (505). The cradle (505) of the charger (500) of the present disclosure has a shape (see concave power providing surface (515)) that mates with the shape of a wearable device of the present disclosure, such as the device of FIGS. 40-45.

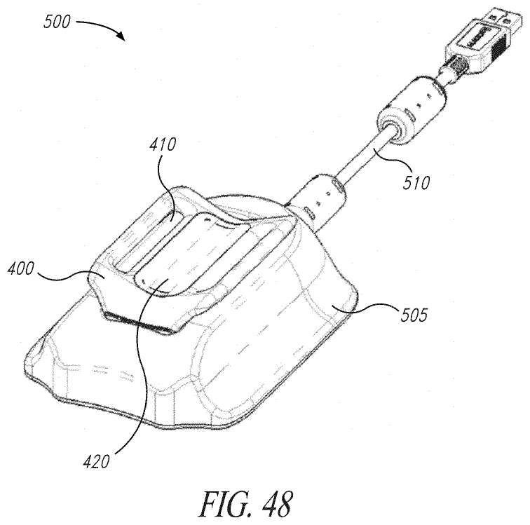

FIG. 48 shows the mating of the cradle (505) of the charger (500) of FIGS. 45-47 with the wearable device of FIGS. 40-42, where such mating is advantageous to create proper alignment between the charger and the wearable device to achieve effective charging of the wearable device by the charger. In FIG. 48 the power receiving surface (418 of FIG. 40, not shown in FIG. 48) of the wearable device (400) has mated to a complementarily contoured power transmitting surface (515) of the cradle (505) of the charger 500). Thus, in one embodiment the present disclosure provides a system comprising a wearable device of the present disclosure and a charger for the wearable device. The charger provides power to the wearable device, thereby replacing power that is consumed by the wearable device during its operation. In one embodiment the charger includes a cradle and a power cord (also referred to as a cable or a power cable), where the cradle is contoured to conform to a shape of the wearable device, so that the cradle mates to a portion of the wearable device and holds the wearable device in a secure position during charging.

Instead of wireless charging with a cradle as described herein, in one embodiment the wearable device is configured to accommodate a wired charger (i.e., power cord connects directly to the wearable to recharge).

In one embodiment the present disclosure provides a device for measuring kinematic movement. The device comprises a housing configured to be securely held to an outer surface of a limb, e.g., a lower leg, of an animal. The device also comprises a plurality of electrical components contained within the housing, where the plurality of electrical components comprises (a) a first sensor configured to sense movement of the limb, e.g., lower leg, and obtain a periodic measure of the movement of the limb and generate a first signal that reflects the periodic measure of the movement, and (b) a second sensor configured to sense movement of the limb, e.g., lower leg and obtain a continuous measure of the movement of the limb and generate a second signal that reflects the continuous measure of the movement. The periodic measure of movement may occur on a regular basis with an interval of a second or more (e.g., at least 2 seconds, or 5 seconds, or 10 seconds) between measurements. The periodic measure of movement may be useful in determining when the subject is making significant movement rather than, e.g., sitting down. The continuous measure of movement may occur for a period of many seconds, e.g., at least 5 seconds, or at least 10 seconds, or at least 15 seconds or at least 20 seconds. During continuous measure of movement, the sensor may obtain data at a sampling rate of between 24 Hz and 1600 Hz, e.g., between 50 Hz and 800 Hz. The device also comprises a memory configured to store data corresponding to the second signal but not the first signal. The device also comprises a telemetry circuit configured to transmit data corresponding to the second signal stored in the memory. The device also comprises a battery configured to provide power to the plurality of electrical components. Optionally, the device the housing of the device is attached to a strap that goes around a limb of a subject, e.g., the lower leg of the subject, to secure the housing to the outer surface of the limb. Optionally, the housing of the device comprises a region with a polymeric surface and the telemetry circuit comprises an antenna that is positioned under the polymeric surface of the housing, to allow transmission of the data corresponding to the second signal through the polymeric surface and to a location separate from the device. Optionally, the telemetry circuit of the device is configured to communicate with a second device via a short range network protocol, such as the medical implant communication service (MICS), the medical device radio communications service (MedRadio), or some other wireless communication protocol such as Bluetooth.

In one embodiment, the intelligent implant is an implanted or implantable medical device having an implantable reporting processor arranged to perform the functions as described herein. The intelligent implant may perform one or more of the following exemplary actions in order to characterize the post-implantation status of the intelligent implant: identifying the intelligent implant or a portion of the intelligent implant, e.g., by recognizing one or more unique identification codes for the intelligent implant or a portion of the intelligent implant; detecting, sensing and/or measuring parameters, which may collectively be referred to as monitoring parameters, in order to collect operational, kinematic, or other data about the intelligent implant or a portion of the intelligent implant and wherein such data may optionally be collected as a function of time; storing the collected data within the intelligent implant or a portion of the intelligent implant; and communicating the collected data and/or the stored data by a wireless means from the intelligent implant or a portion of the intelligent implant to an external computing device. The external computing device may have or otherwise have access to at least one data storage location such as found on a personal computer, a base station, a computer network, a cloud-based storage system, or another computing device that has access to such storage.

Non-limiting and non-exhaustive list of embodiments of intelligent implants include components of a total knee arthroplasty (TKA) or partial knee arthroplasty (PKA) system, including a TKA tibial plate, a TKA femoral component, a TKA patellar component, a tibial extension; components of a total hip arthroplasty (THA) or partial hip arthroplasty (PHA) system, including a THA femoral component, the THA acetabular component, components of a total shoulder arthroplasty (TSA) or partial shoulder arthroplasty (PSA) system, ankle and elbow arthroplasty devices, an intramedullary rod for arm or leg breakage repair, a scoliosis rod, a dynamic hip screw, a spinal interbody spacer, a spinal artificial disc, an annuloplasty ring, a heart valve, an intravascular stent, a cerebral aneurysm coil or diverting stent device, a breast implant, a vascular graft and a vascular stent graft.

In some embodiments, a wearable device may be used to obtain a pre-operative or otherwise baseline data set of kinematic data for a particular patient. After implantation of an intelligent implant (such as an implant placed during a TKA procedure), the implant may be used to obtain a post-operative data set of kinematic data. Analysis of the kinematic data, including any of the statistical and/or machine learning analyses described herein, may be further applied to the pre-operative and post-operative data sets separately or in combination, to compare the pre-operative and post-operative conditions of a patient.

Thus, in one aspect the present disclosure provides a method comprising obtaining pre-operative kinematic data from a patient using a wearable device such as disclosed herein, thereafter obtaining post-operative kinematic data from the patient using an implantable device such as disclosed herein, and comparing the pre-operative data to the post-operative data, where analysis of the kinematic data, including any of the statistical and/or machine learning analyses described herein, may be further applied to the pre-operative and post-operative data sets separately or in combination, to compare the pre-operative and post-operative conditions of a patient. In one embodiment the implantable device is implanted in a joint of the patient during a TJA (total joint arthroplasty) or PJA (partial joint arthroplasty), and the wearable device is worn on or near the joint of the patient, where exemplary joints include knee, hip and shoulder. In one embodiment the implantable device is implanted in a knee of the patient during a TKA (total knee arthroplasty) or PKA (partial knee arthroplasty), and the wearable device is worn on or near the knee of the patient. In one embodiment the implantable device is implanted in a hip of the patient during a THA (total hip arthroplasty) or PHA (partial hip arthroplasty), and the wearable device is worn on or near the hip of the patient. In one embodiment the implantable device is implanted in a shoulder of the patient during a TSA (total shoulder arthroplasty) or PSA (partial shoulder arthroplasty), and the wearable device is worn on or near the shoulder of the patient.

“Kinematic data,” as used herein, individually or collectively includes some or all data associated with a particular kinematic device and available for communication outside of the particular kinematic device. For example, kinematic data may include raw data from one or more sensors of a kinematic device, wherein the one or more sensors may include gyroscopes, accelerometers, pedometers, strain gauges, acoustic sensors, and the like that produce data associated with motion, force, torque, tension, pressure, velocity, rotational velocity, acceleration, or other mechanical forces. Kinematic data may also include processed data from one or more sensors, status data, operational data, control data, fault data, time data, scheduled data, event data, log data, and the like associated with the particular kinematic implantable device. In some cases, high resolution kinematic data includes kinematic data from one, many, or all of the sensors of the kinematic implantable device that is collected in higher quantities, resolution, from more sensors, more frequently, or the like. In one embodiment, the kinematic device is an implantable kinematic device. In one embodiment, the kinematic device is an external, wearable kinematic device.

In one embodiment, kinematics refers to the measurement of the positions, angles, velocities, and accelerations of body segments and joints during motion. Body segments are considered to be rigid bodies for the purposes of describing the motion of the body. They include the foot, shank (leg), thigh, pelvis, thorax, hand, forearm, upper-arm and head. Joints between adjacent segments include the ankle (talocrural plus subtalar joints), knee, hip, wrist, elbow, shoulder, and spine. Position describes the location of a body segment or joint in space, measured in terms of distance, e.g., in meters. A related measurement called displacement refers to the position with respect to a starting position. In two dimensions, the position is given in Cartesian co-ordinates, with horizontal followed by vertical position. In one embodiment, a kinematic implant or kinematic wearable device obtains kinematic data, and optionally obtains only kinematic data.

“Kinematic element” (also referred to herein as “element”) refers to points, marks, peaks, regions, etc. within kinematic data corresponding to motion activity of a body part that are associated with a kinematic aspect of such motion. For example, elements, e.g., fiducial points, in a time-series waveform of rotational velocity may corresponds to inflection points of the waveform that represent zero velocity of the body part, or other points that represent maximum velocity.

“Kinematic feature” refers to metrics or variables that may be derived from elements. For example, continuing with the time-series waveform, metrics such as time intervals between points, ratios of time intervals, peak-to-peak elevations of points, elevation differentials of points, etc. may be derived from the elements. Kinematic features also refers to kinematic parameters, such as cadence, stride length, walking speed, tibia range of motion, knee range of motion, step count and distance traveled, that may be derived from kinematic data. Kinematic features also refers to visual representations of kinematic data, including for example time-series waveforms, spectral distribution graphs, and spectrograms.

“Outcome” refers to a diagnostic outcome or prognostic outcome of interest in relation to a kinematic device and the patient with which the device is associated. Outcomes may include, for example, clinical outcomes such as a movement classification (e.g., patient is walking normally or abnormally), a recovery state (e.g., patient is fully recovered or partially recovered), and a medical condition state (e.g., patient has an infection, or is likely to develop an infection, patient is in pain or is likely to experience pain), device conditions (e.g., implant is loosening). Outcomes may include, for example, economic outcomes, e.g., patient cost of full recovery is likely to cost a certain amount.

“Sensor” refers to a device that can be utilized to do one or more of detect, measure and/or monitor one or more different aspects of a body (anatomy, physiology, metabolism, and/or function/mechanics) and/or one or more aspects of the orthopedic device or implant. Representative examples of sensors suitable for use within the present disclosure include, for example, fluid pressure sensors, fluid volume sensors, contact sensors, position sensors, pulse pressure sensors, blood volume sensors, blood flow sensors, acoustic sensors (including ultrasound), chemistry sensors (e.g., for blood and/or other fluids), metabolic sensors (e.g., for blood and/or other fluids), accelerometers, gyroscopes, magnetometers, mechanical stress sensors and temperature sensors. Within certain embodiments the sensor can be a wireless sensor, or, within other embodiments, a sensor connected to a wireless microprocessor. Within further embodiments one or more (including all) of the sensors can have a Unique Sensor Identification number (“USI”) which specifically identifies the sensor. In certain embodiments, the sensor is a device that can be utilized to measure in a quantitative manner, one or more different aspects of a body (anatomy, physiology, metabolism, and/or function/mechanics) and/or one or more aspects of the orthopedic device or implant. In certain embodiments, the sensor is an accelerometer that can be utilized to measure in a quantitative manner, one or more different aspects of a body (e.g., function) and/or one or more aspects of the orthopedic device or implant (e.g., alignment in the patient).

A wide variety of sensors (also referred to as Microelectromechanical Systems or “MEMS”, or Nanoelectromechanical Systems or “NEMS”, and BioMEMS or BioNEMS) can be utilized within the present disclosure. Representative patents and patent applications include U.S. Pat. Nos. 7,383,071, 7,450,332; 7,463,997, 7,924,267 and 8,634,928, and U.S. Publication Nos. 2010/0285082, and 2013/0215979. Representative publications include “Introduction to BioMEMS” by Albert Foch, CRC Press, 2013; “From MEMS to Bio-MEMS and Bio-NEMS: Manufacturing Techniques and Applications by Marc J. Madou, CRC Press 2011;” Bio-MEMS: Science and Engineering Perspectives, by Simona Badilescu, CRC Press 2011; “Fundamentals of BioMEMS and Medical Microdevices” by Steven S. Saliterman, SPIE—The International Society of Optical Engineering, 2006; “Bio-MEMS: Technologies and Applications”, edited by Wanjun Wang and Steven A. Soper, CRC Press, 2012; and “Inertial MEMS: Principles and Practice” by Volker Kempe, Cambridge University Press, 2011; Polla, D. L., et al., “Microdevices in Medicine,” Ann. Rev. Biomed. Eng. 2000, 02:551-576; Yun, K. S., et al., “A Surface-Tension Driven Micropump for Low-voltage and Low-Power Operations,” J. Microelectromechanical Sys., 11:5, October 2002, 454-461; Yeh, R., et al., “Single Mask, Large Force, and Large Displacement Electrostatic Linear Inchworm Motors,” J. Microelectromechanical Sys., 11:4, August 2002, 330-336; and Loh, N. C., et al., “Sub-10 cm3 Interferometric Accelerometer with Nano-g Resolution,” J. Microelectromechanical Sys., 11:3, June 2002, 182-187; all of the above of which are incorporated by reference in their entirety.

“Biomarker,” as used herein, refers to an objective indication of a medical state, which can be measured accurately and reproducibly, and used to monitor and treat progression of the medical state. Biomarkers individually or collectively include physiological measurements, anatomical measurements, metabolic measurements, and functional/mechanical measurements, such as may be provided by the above-described sensors. Biomarkers also include quantifiable aspects or characteristics of the aforementioned measurements. For example, as disclosed herein biomarkers include kinematic features, e.g., intervals, ratios of intervals, peak-to-peak elevation, and elevation differentials derived from elements identified in kinematic data corresponding to motion activity. Biomarkers also include kinematic features corresponding to kinematic parameters, such as cadence, stride length, walking speed, tibia range of motion, knee range of motion, step count and distance traveled, that may be derived from kinematic data.

“Dataset,” as used herein, individually or collectively includes some or all data or information associated with a particular patient with a kinematic implantable or wearable device. For example, a patient dataset may include kinematic data (as described above) for the patient, biomarkers (as described above) for the patient, medical data of the patient, and demographic data of the patient. Medical data may include information related to the kinematic implantable device implanted in the patient, such as device type information, device component information, manufacturer information, device configuration information (e.g., sensor types, sensor parameters or settings, and sampling schedule), hospital and surgeon performing the surgery, any complications or notes from the surgery, and the date the device was implanted in the patient. Demographic data may include information related to the patient, such as date of birth, gender, ethnicity, geographic location.

### Intelligent Implants

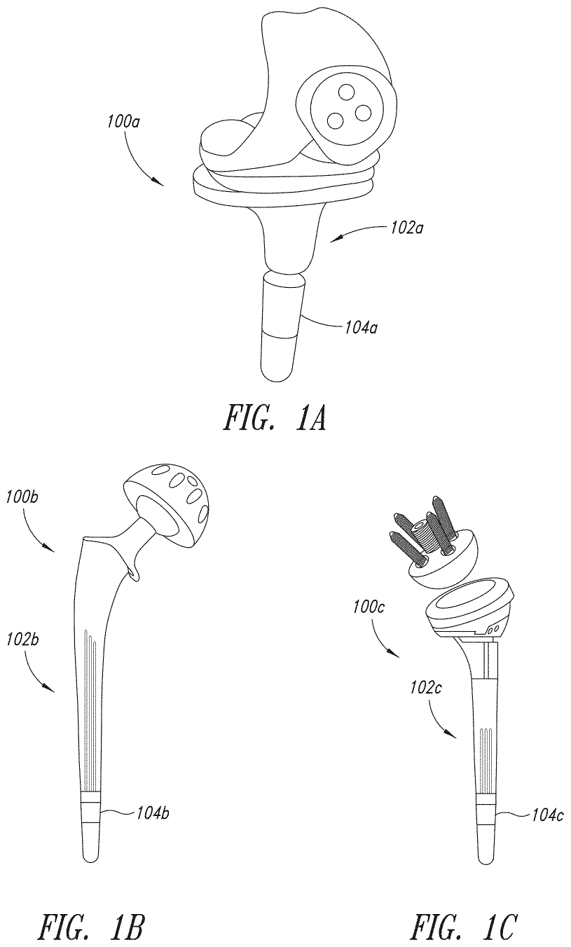

With reference to FIGS. 1A-1C, the present disclosure provides intelligent implants 100a, 100b, 100c, e.g., an implantable medical device 102a, 102b, 102c with an implantable reporting processor (IRP) 104a, 104b, 104c, that may be utilized to monitor and report the status and/or activities of the implant itself, and the patient in which the intelligent implant is implanted. In some embodiments, the intelligent implant 100a, 100b, 100c is part of an implant system, e.g., a total or partial joint arthroplasty system, that replaces a joint of a patient and allows the patient to have the same, or nearly the same, mobility as would have been afforded by a healthy joint. Examples of joint arthroplasty systems with intelligent implants 100a, 100b, 100c include partial and total knee arthroplasty systems (FIG. 1A), partial and total hip arthroplasty systems (FIG. 1B), and partial and total shoulder arthroplasty systems (FIG. 1C). It should be understood that in one embodiment the IPR may be a component of a wearable device of the present disclosure and that reference to an IPR in an implantable device as described herein may also provide a description of an IPR contained as part of a wearable device of the present disclosure.

When the intelligent implant 100a, 100b, 100c is located adjacent to or included in a component of an implant system that replaces a joint, the intelligent implant can collect and provide datasets of kinematic data that may be processed and analyzed to assess patient recovery, potential complications, and implant integrity. For example, as disclosed herein, analysis of kinematic data may determine how well a patient is recovering from surgery. Analysis of kinematic data may also detect implant complications, e.g., micromotion, contracture, aseptic loosening, and infection, that may require an early intervention, such as bracing, changing one or more components of the implant, administration of systemic or local antibiotics, or manipulation of the extremity and implant. The intelligent implant can also monitor displacement or movement of the component or implant system. Examples of joint replacement implant systems in which the intelligent implants disclosed herein may be incorporated, are described in PCT Publication Nos. WO 2014/144107, WO 2014/209916, WO 2016/044651, WO 2017/165717, and WO 2020/247890, the disclosures of which are incorporated herein.

With reference to FIG. 1A, for the intelligent implant 100a embodiment described in detail in this disclosure, the implantable medical device 102a is a tibial extension of a knee replacement system for a partial or total knee arthroscopy (TKA). The IRP 104a of the intelligent implant 100a, which extends into the tibia, can monitor and provide data that can be used to characterize movement of the knee implant and by proxy, movement of the body part in which the intelligent implant is implanted. For example, the IRP 104a may provide data on the movement of the patient's leg. In general, there are three types of three-dimensional motion or movement that the intelligent implant 100a can detect within and around a joint: core gait (or limb mobility in the case of a shoulder or elbow arthroplasty), macroscopic instability, and microscopic instability. Details of these types of motion are described in detail in PCT Publication Nos. WO 2017/165717 and WO 2020/247890.

In other embodiments, the implantable medical device may be adjacent to, or included in, a partial or total hip replacement prosthesis including one or more of a femoral stem, femoral head and an acetabular implant, and an IRP. In another embodiment, the implantable medical device may be adjacent to, or included in, a partial or total shoulder replacement prosthesis including one or more of a humeral stem, humeral head and a glenoid implant, and an IRP. Examples of a spinal implant that includes pedicle screws, spinal rods, spinal wires, spinal plates, spinal cages, artificial discs, bone cement, as well as combinations of these (e.g., one or more pedicle screws and spinal rods, one or more pedicle screws and a spinal plate).

### Tibial Extension—Structure and Assembly

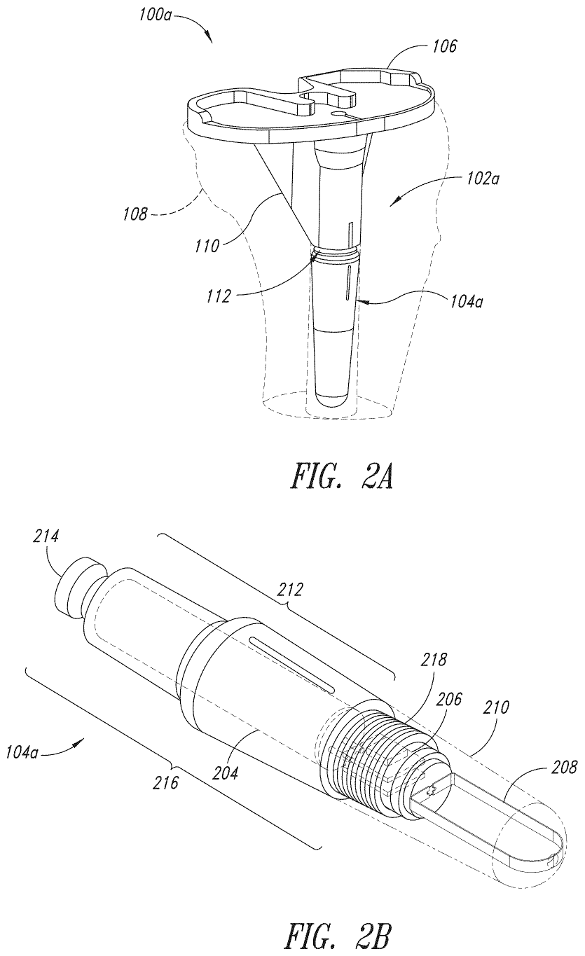

With reference to FIG. 2A, an embodiment of an intelligent implant 100a corresponding to a tibial extension includes an implantable medical device 102a and an implantable reporting processor (IRP) 104a. The implantable medical device 102a includes a tibial plate 106 physically attached to an upper surface of a tibia 108 and support structure 110 that extends downward from the tibial plate 106. The support structure 110 includes a receptacle 112 configured to receive the IRP 104a. Prior to, or during the implant procedure, the IRP 104a is physically attached to the support structure 110 and is implanted into the tibia 108.

With reference to FIG. 2B, in some embodiments the IRP 104a includes an outer casing or housing that encloses a power component (battery) 204, an electronics assembly 206, and an antenna 208. The housing of the implantable reporting processor 104 includes a radome 210 or cover and an extension 216. The extension 216 includes a central section 212, an upper coupling section 214, and a lower coupling section 218 with which the cover 210 is configured to couple.

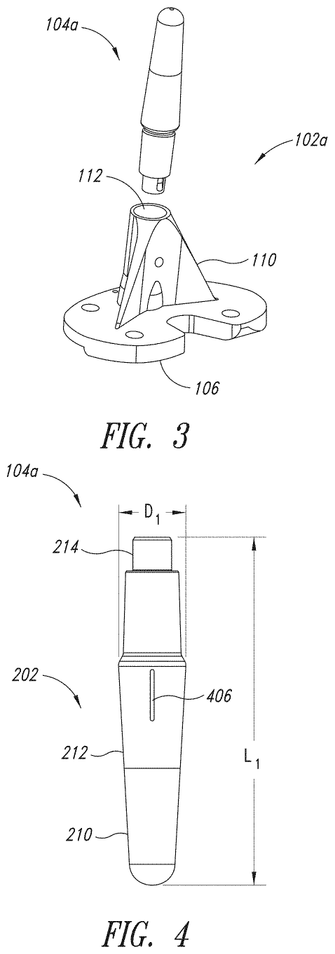

With additional reference to FIGS. 3 and 4, the housing 202 has a length L₁ of about 73 millimeters (mm), and has a diameter D₁ of about 14 mm at its widest cross section. In various embodiments, an implantable reporting processor 104 may have a length L₁ selected from 70 mm, or 71 mm, or 72 mm, or 73 mm, or 74 mm, or 75 mm, or 76 mm, or 77 mm, or 78 mm, or 79 mm, or 80 mm, or 85 mm, or 90 mm, or 95 mm, or 100 mm, and a range provided by selecting any two of these L₁ values. In various embodiments, an implantable reporting processor 104 may have a diameter D₁ at its widest cross-section of 5 mm, or 13 mm, or 14 mm, or 15 mm, or 16 mm, or 17 mm, or 18 mm, or 19 mm, or 20 mm, or 22 mm, or 24 mm, or 26 mm, or 28 mm, or 30 mm, and range provided by selecting any two of the D₁ values. It should be noted that the term diameter is used in a broad sense to refer to a maximum cross-sectional distance, where that cross-section need not be an exact circle, but may be other shapes such as oval, elliptical, or even 4-, 5- or 6-sided.

The radome 210 covers and protects the antenna 208, which allows the implantable reporting processor 104 to receive and transmit data/information (hereinafter “information”). The radome 210 can be made from any material, such as plastic or ceramic, which allows radiofrequency (RF) signals to propagate through the radome with acceptable levels of attenuation and other signal degradation. In some embodiments the radome 210 is comprised of polyether ether ketone (PEEK).

The central section 212 and the upper coupling section 214, which are integral with one another, cover and protect the electronics assembly 206 and the battery 204, and can be made from any suitable material, such as metal, plastic, or ceramic. Furthermore, the central section 212 includes an alignment mark 406, which is configured to align with a corresponding alignment mark (not shown in FIGS. 3 and 4) on the outside of the receptacle 112. Aligning the alignment mark 406 with the mark on the receptacle 112 when the tibial component 102a of the knee implant is implanted ensures that the implantable reporting processor 104a is in a desired orientation relative to the support structure 110.

The upper coupling section 214 is sized and otherwise configured to fit into the receptacle 112 of the support structure 110. The fit may be snug enough so that no securing mechanism (e.g., adhesive, set-screw) is needed, or the upper coupling section 214 can include a securing mechanism, such as threads, clips, and/or a set-screw (not shown) and a set-screw engagement hole, for attaching and securing the implantable reporting processor 104a to the support structure 110.

The primary components of the implantable reporting processor 104a include the battery 204, the electronics assembly 206, and the antenna 208. The battery 204 is configured to power the electronic circuitry of the implantable reporting processor 104a over a significant portion (e.g., 1-15+ years, e.g., 10 years, or 15 years), or the entirety (e.g., 18+ years), of the anticipated lifetime of the implantable reporting processor.

In some embodiments, the battery 204 has a lithium-carbon-monofluoride (LiCFx) chemistry, a cylindrical housing or cylindrical container, a cathode terminal, and an anode terminal, which is a plate that surrounds the cathode terminal. LiCFx is a non-rechargeable (primary) chemistry, which is advantageous for maximizing the battery energy storage capacity. The cathode terminal makes conductive contact with an internal cathode electrode and couples to the cylindrical container using a hermetic feed-through insulating material of glass or ceramic. The use of the hermetic feed through prevents leakage of internal battery materials or reactive products to the exterior battery surface. Furthermore, the glass or ceramic feed-through material electrically insulates the cathode terminal from the cylindrical container, which makes conductive contact with the internal anode electrode. The anode terminal is welded to the cylindrical container. By locating the cathode terminal and the anode terminal on the same end of the battery 204, both terminals can be coupled to the electronics assembly 206 without having to run a lead, or other conductor, to the opposite end of the battery.

The container can be formed from any suitable material, such as titanium or stainless steel, and can have any configuration suitable to limit expansion of the battery 204 as the battery heats during use. Because the battery 204 is inside of the extension 216, if the battery were to expand too much, it could crack the container or the extension 216, or irritate the subject's tibia or other bodily tissue.

With its LiCFx chemistry, the battery 204 can provide, over its lifetime, about 360 milliampere hours (mAh) at 3.7 volts (V), although one can increase this output by about 36 mAh for each 5 mm of length added to the battery (similarly, one can decrease this output by about 36 mAh for each 5 mm of length subtracted from the battery). It is understood that other battery chemistries can be used if they can achieve the appropriate power requirements for a given application subject to the size and longevity requirements of the application. Some additional potential battery chemistries include, but are not limited to, Lithium ion (Li-ion), Lithium Manganese dioxide (Li—MnO2), silver vanadium oxide (SVO), Lithium Thionyl Chloride (Li—SOCl2), Lithium iodine, and hybrid types consisting of combinations of the above chemistries such as CFx-SVO.

The electronics assembly 206 includes one or more sensors and a processor configured to receive and process information from the sensors relating to the state and functioning of the implantable reporting processor 104 and the state of the patient within which the implantable reporting processor is implanted. The electronics assembly 206 is further configured to transmit the processed information to an external device through the antenna 208.

The electronics assembly 206 is coupled physically and electrically to the antenna 208 through terminals on the antenna terminal board 208, and to the power component (e.g., battery) through terminals on the battery terminal board. The PCBs may include an Inertial Measurement Unit (IMU) integrated circuit, a Real-Time Clock (RTC) integrated circuit, a memory integrated circuit (Flash), and other circuit components on one side, and a microcontroller (MCU) integrated circuit, a radio transmitter (RADIO) integrated circuit, and other circuit components on the other side. In any event, the folded electronics assembly 206 provides a compact configuration that conserves a significant amount of physical space in the implantable reporting processor.

The antenna 208 is designed to transmit information generated by the electronics assembly 206 to a remote destination outside of the body of a subject in which the intelligent implant is implanted, and to receive information from a remote source outside of the subject's body.

In some embodiments, the implantable reporting processor 104a further comprises an epoxy material that encapsulates the antenna 208 within the cover 210. The epoxy material may be medical grade silicone. Encapsulating the antenna 208 increases structural rigidity of the implantable reporting processor 104a, and isolates the antenna from tissue and body fluid.

Thus disclosed is an IRP 104a structure wherein all active electronics and the battery 204 are contained within a hermetic assembly 126. The ground reference potential of the battery 204 is physically welded to the lower shroud 606 and the extension 216. By virtue of the intimate contact between the extension 216 and the tibial plate 106 with surrounding tissue, the IRP 104 ground reference potential is equal to the body tissue potential (electrically neutral with surrounding tissue). Within the hermetic assembly 126, both the battery 204 reference potential (GND) and the battery positive terminal potential (VBATT) are routed throughout the electronics assembly 206 to power the electronic components. The feedthrough 612 provides connections between the electronics inside the hermetic assembly 126 and the radio loop antenna 208 outside the hermetic assembly. The antenna 208 is a conductive loop formed of platinum-iridium (PtIr=90/10) ribbon with one end connected to the radio transceiver and the other end connected to the battery reference potential (GND). The conductive loop antenna 208 provides a magnetic loop, e.g., AC signal in a conductive loop generates magnetic field. The antenna 208 is encapsulated by the PEEK radome 210 and epoxy backfill, both of which are electrically non-conductive. The antenna 208 is the only electrically active component of the IRP 104 outside the hermetic assembly 126 and under normal operating conditions is insulated by the epoxy backfill and PEEK radome from interacting electrically with surrounding tissue.

### Tibial Extension—Electronics

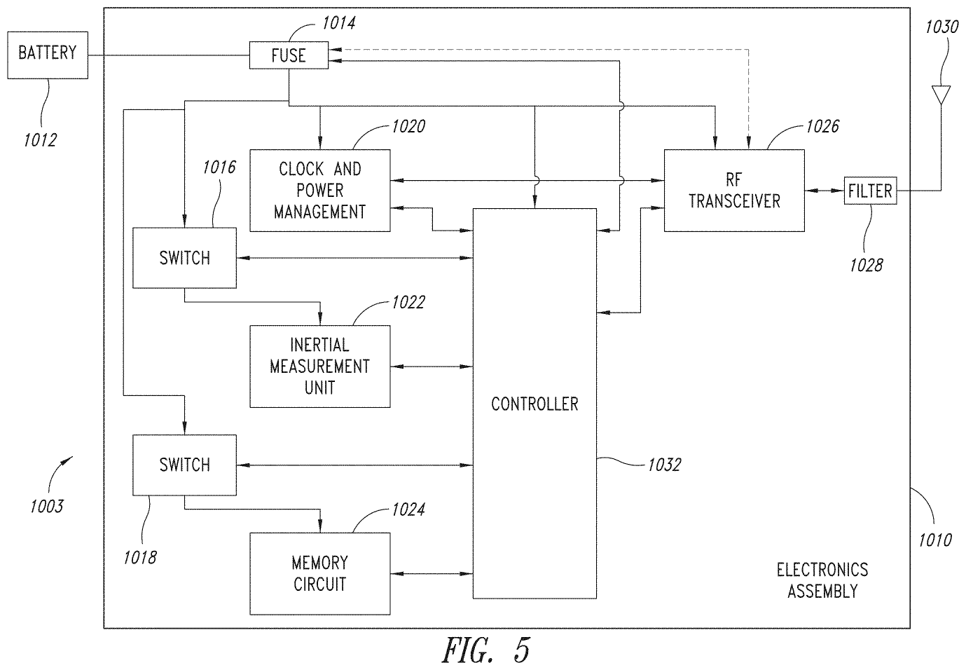

With reference to FIG. 5, as previously described, an embodiment of an implantable reporting processor 1003 includes an electronics assembly 1010, a battery 1012 or other suitable implantable power source, and an antenna 1030. The electronics assembly 1010 includes a fuse 1014, switches 1016 and 1018, a clock generator and clock and power management circuit 1020, an inertial measurement unit (IMU) 1022, a memory circuit 1024, a radio-frequency (RF) transceiver 1026, an RF filter 1028 and a controller 1032. Examples of some or all of these components are described elsewhere in this application and in PCT Publication Nos. WO 2017/165717 and WO 2020/247890, which are incorporated by reference.

The battery 1012 can be any suitable battery, such as a Lithium Carbon Monofluoride (LiCFx) battery, or other storage cell configured to store energy for powering the electronics assembly 1010 for an expected lifetime (e.g., 5-25+ years) of the kinematic implant.

The fuse 1014 can be any suitable fuse (e.g., permanent) or circuit breaker (e.g., resettable) configured to prevent the battery 1012, or a current flowing from the battery, from injuring the patient and damaging the battery and one or more components of the electronics assembly 1010. For example, the fuse 1014 can be configured to prevent the battery 1012 from generating enough heat to burn the patient, to damage the electronics assembly 1010, to damage the battery, or to damage structural components of the kinematic implant.

The switch 1016 is configured to couple the battery 1012 to, or to uncouple the battery from, the IMU 1022 in response to a control signal from the controller 1032. For example, the controller 1032 may be configured to generate the control signal having an open state that causes the switch 1016 to open, and, therefore, to uncouple power from the IMU 1022, during a sleep mode or other low-power mode to save power, and, therefore, to extend the life of the battery 1012. Likewise, the controller 1032 also may be configured to generate the control signal having a closed state that causes the switch 1016 to close, and therefore, to couple power to the IMU 1022, upon “awakening” from a sleep mode or otherwise exiting another low-power mode. Such a low-power mode may be for only the IMU 1022 or for the IMU and one or more other components of the implantable.

The switch 1018 is configured to couple the battery 1012 to, or to uncouple the battery from, the memory circuit 1024 in response to a control signal from the controller 1032. For example, the controller 1032 may be configured to generate the control signal having an open state that causes the switch 1018 to open, and, therefore, to uncouple power from the memory circuit 1024, during a sleep mode or other low-power mode to save power, and, therefore, to extend the life of the battery 1012. Likewise, the controller 1032 also may be configured to generate the control signal having a closed state that causes the switch 1018 to close, and therefore, to couple power to the memory circuit 1024, upon “awakening” from a sleep mode or otherwise exiting another low-power mode. Such a low-power mode may be for only the memory circuit 1024 or for the memory circuit and one or more other components of the electronics assembly 1010.

The clock and power management circuit 1020 can be configured to generate a clock signal for one or more of the other components of the electronics assembly 1010, and can be configured to generate periodic commands or other signals (e.g., interrupt requests) in response to which the controller 1032 causes one or more components of the implantable circuit to enter or to exit a sleep, or other low-power, mode. The clock and power management circuit 1020 also can be configured to regulate the voltage from the battery 1012, and to provide a regulate power-supply voltage to some or all of the other components of the electronics assembly 1010.

The IMU 1022 has a frame of reference with coordinate x, y, and z axes, and can be configured to measure, or to otherwise quantify, acceleration (acc) that the IMU experiences along each of the x, y, and z axes, using a respective one of three accelerometers associated with the IMU. The IMU 1022 can also be configured to measure, or to otherwise quantify, angular velocity (Ω) that the IMU experiences about each of the x, y, and z axes, using a respective one of three gyroscopes associated with the IMU. Such a configuration of the IMU 1022 is at least a six-axis configuration, because the IMU 1022 measures six unique quantities, accₓ(g), acc_{y}(g), acc_{z}(g), Ωₓ(dps), Ω_{y}(dps), and Ω_{z}(dps). Alternatively, the IMU 1022 can be configured in a nine-axis configuration, in which the IMU can use gravity to compensate for, or to otherwise correct for, accumulated errors in accₓ(g), acc_{y}(g), acc_{z}(g), Ωₓ(dps), Ω_{y}(dps), and Ω_{z}(dps). But in an embodiment in which the IMU measures acceleration and angular velocity over only short bursts (e.g., 0.10-100 seconds(s)), for many applications accumulated error typically can be ignored without exceeding respective error tolerances.

The IMU 1022 can include a respective analog-to-digital converter (ADC) for each of the three accelerometers and three gyroscopes. Alternatively, the IMU 1022 can include a respective sample-and-hold circuit for each of the three accelerometers and gyroscopes, and as few as one ADC that is shared by the accelerometers and gyroscopes. Including fewer than one ADC per accelerometer and gyroscope can decrease one or both of the size and circuit density of the IMU 1022, and can reduce the power consumption of the IMU. But because the IMU 1022 includes a respective sample-and-hold circuit for each accelerometer and each gyroscope, samples of the analog signals generated by the accelerometers and the gyroscopes can be taken at the same or different sample times, at the same or different sample rates, and with the same or different output data rates (ODR).

The memory circuit 1024 can be any suitable nonvolatile memory circuit, such as EEPROM or FLASH memory, and can be configured to store data written by the controller 1032, and to provide data in response to a read command from the controller.

The RF transceiver 1026 can be a conventional transceiver that is configured to allow the controller 1032 (and optionally the fuse 1014) to communicate with a base station (not shown in FIG. 4) configured for use with the kinematic implantable device. For example, the RF transceiver 1026 can be any suitable type of transceiver (e.g., Bluetooth, Bluetooth Low Energy (BTLE), and WiFi®), can be configured for operation according to any suitable protocol (e.g., MICS, ISM, Bluetooth, Bluetooth Low Energy (BTLE), and WiFi®), and can be configured for operation in a frequency band that is within a range of 1 MHz-5.4 GHz, or that is within any other suitable range.

The RF filter 1028 can be any suitable bandpass filter, such as a surface acoustic wave (SAW) filter or a bulk acoustic wave (BAW) filter. In some embodiment, the RF filter 1028 includes multiple filters and other circuitry to enable dual-band communication. For example, the RF filter 1028 may include a bandpass filter for communications on a MICS channel, and a notch filter for communication on a different channel, such as a 2.45 GHz.

The antenna 1030 can be any antenna suitable for the frequency band in which the RF transceiver 1026 generates signals for transmission by the antenna, and for the frequency band in which a base station generates signals for reception by the antenna. In some embodiments the antenna 1030 is configured as a flat ribbon loop antenna as described above with reference to FIGS. 2A-2B.

The controller 1032, which can be any suitable microcontroller or microprocessor, is configured to control the configuration and operation of one or more of the other components of the electronics assembly 1010. For example, the controller 1032 is configured to control the IMU 1022 to take measurements of movement of the implantable medical device with which the electronics assembly 1010 is associated, to quantify the quality of such measurements (e.g., is the measurement “good” or “bad”), to store measurement data (also referred to herein as “kinematic data”) generated by the IMU in the memory 1024, to generate messages that include the stored data as a payload, to packetize the messages, to provide the message packets to the RF transceiver 1026 for transmission to an external device, e.g. a base station.

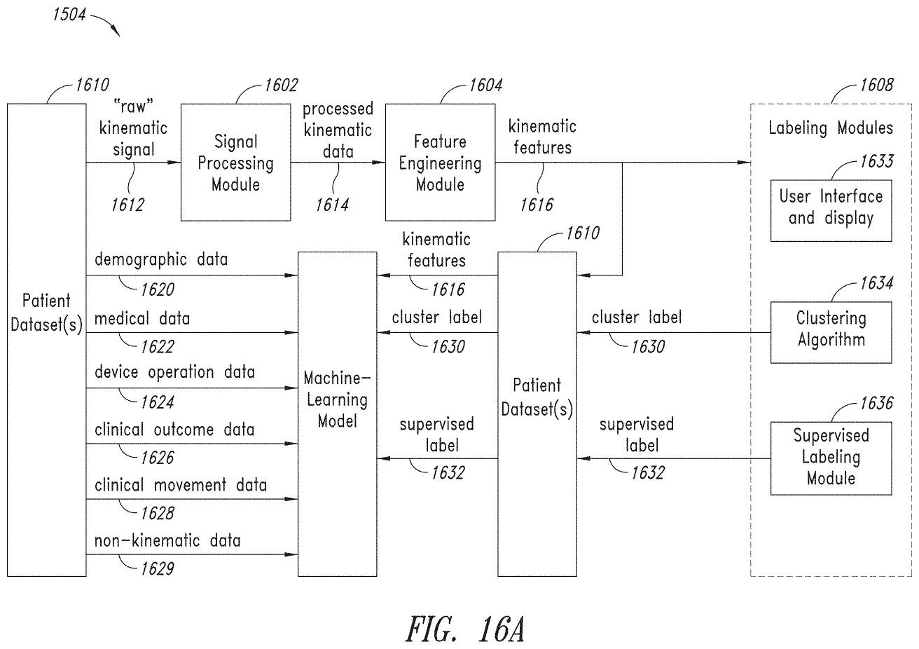

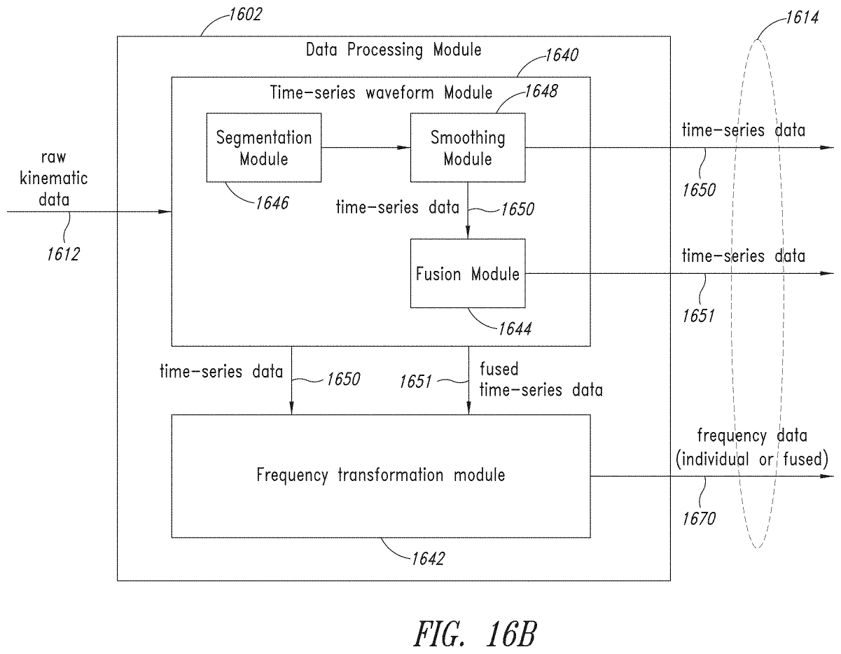

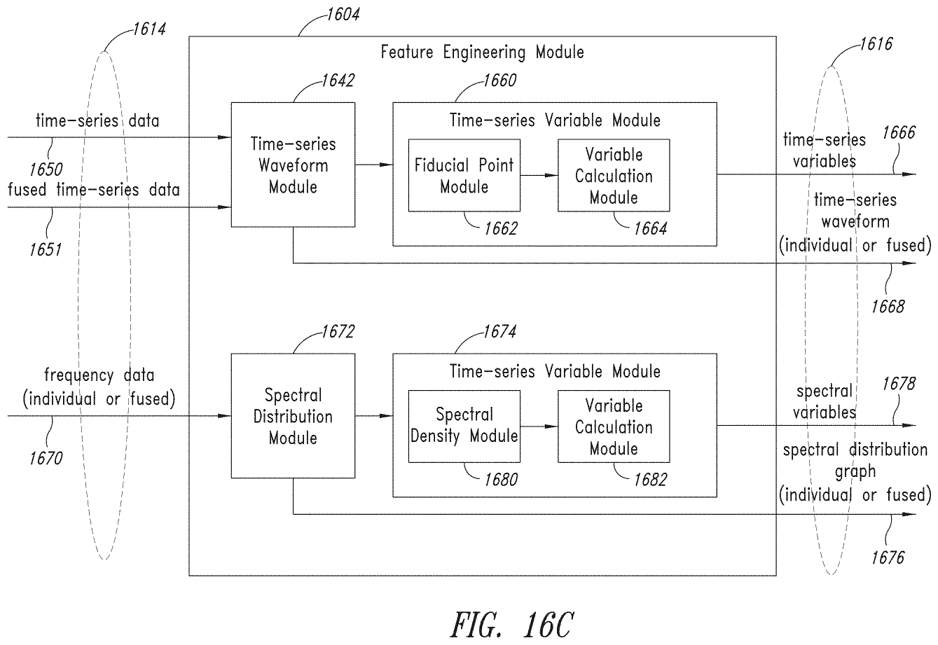

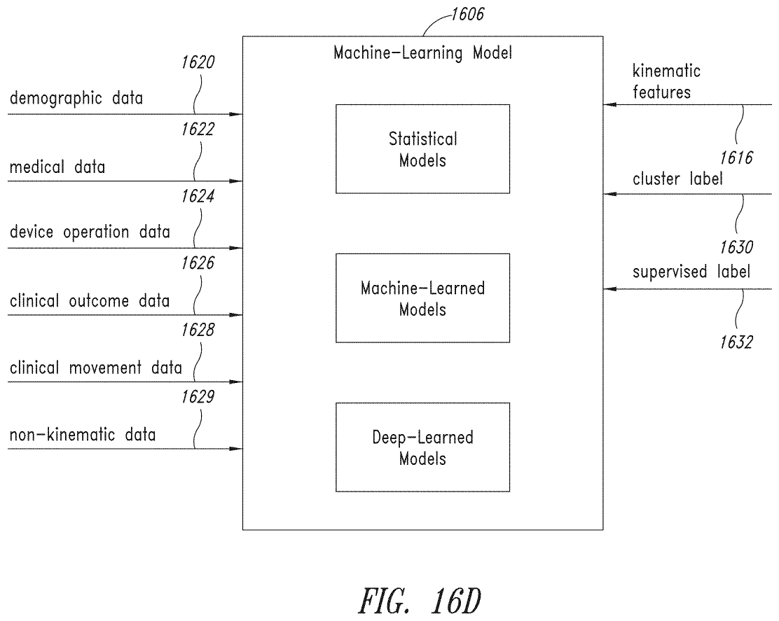

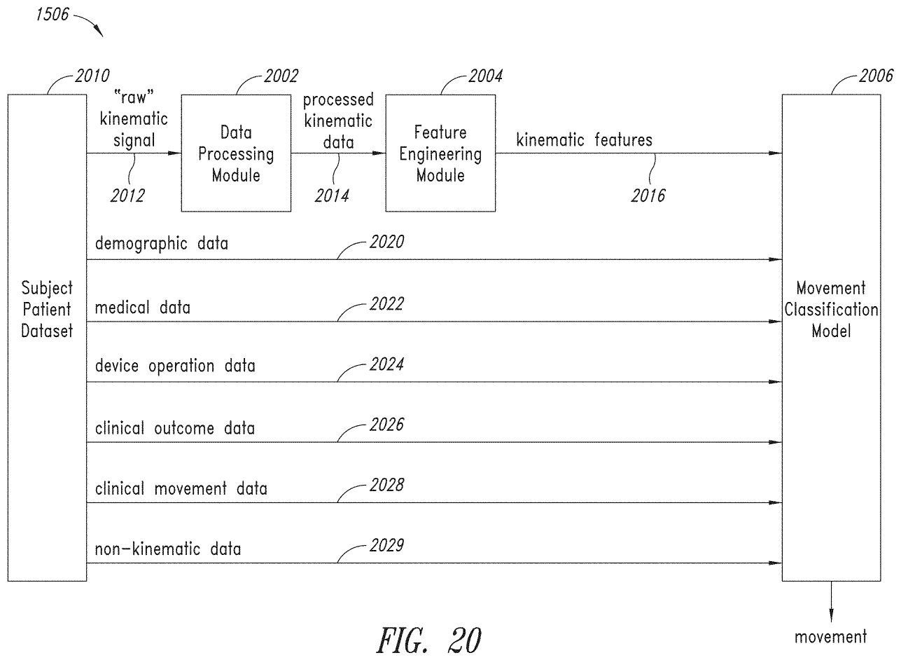

The controller 1032 may include a patient movement classification model (not shown) that is configured to process kinematic data generated by the IMU 1022 to classify the movement of a patient body part, e.g., tibia, hip, shoulder, etc., with which the IMU is associated. The patient movement classification model—also referred to simply as a “movement classification model” for brevity—may correspond to the classification apparatus described further below with reference to FIG. 20. The movement classification model may, for example, process a bout of kinematic data obtained by the IMU 1022 to identify movement activity of the body part, and to classify such activity as one of a normal movement or an abnormal movement or any other movement classification type that the classification model is trained to identify. Example movement classification types are described further below with reference to FIG. 16A-19C. The controller 1032 stores the identified classification type with the corresponding kinematic data and includes it in the payload of the message that is eventually transmitted to an external device.

The controller 1032 may be configured to execute commands received from an external device via the antenna 1030, the RF filter 1028, and the RF transceiver 1026. For example, the controller 1032 can be configured to receive configuration data from a base station, and to provide the configuration data to the component of the electronics assembly 1010 to which the base station directed the configuration data. If the base station directed the configuration data to the controller 1032, then the controller is configured to configure itself in response to the configuration data. The controller 1032 may also be configured to execute data sampling by the IMU 1022 in accordance with one or more programmed sampling schedules, or in response to an on-demand data sampling command received from a base station. For example, as described later below, the IRP 104 may be programmed to operate in accordance with a master sampling schedule and a periodic, e.g., daily, sampling schedule.

### Inertial Measurement Unit Sensing

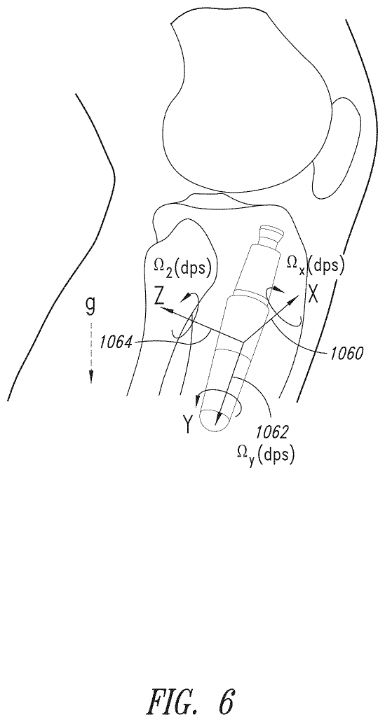

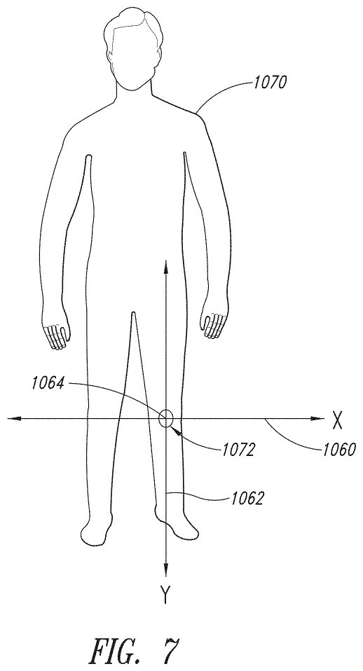

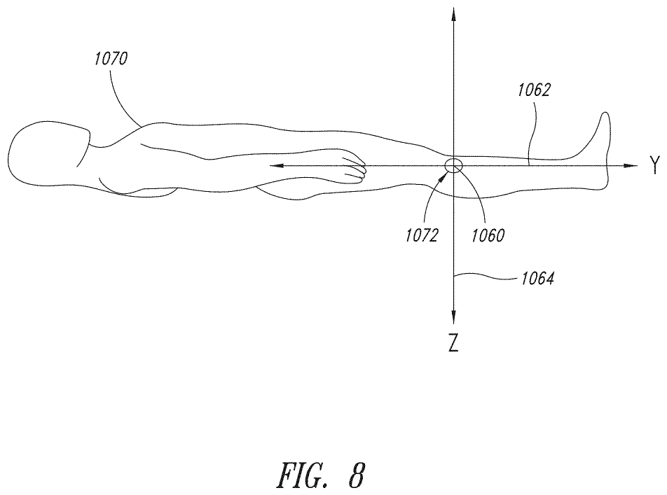

FIG. 6 is a perspective view of the IRP 104a of FIG. 4 implanted in a tibia of a left knee of a patient, and showing a set of coordinate axes 1060, 1062, and 1064 associated with an IMU 1022 of the IRP. The IMU 1022 may be, for example, a Bosch BMI 160 small, low-power, IMU. With respect to the anatomy of the patient, the positive portion of the x-axis 1060 extends in the direction outward from the leg. In other words, the positive portion of the x-axis 1060 extends away from the other leg of the patient. The positive portion of the y-axis 1062 extends in the direction downward toward the foot of the patient. The positive portion of the z-axis 1064 extends in the direction outward from the back of the knee of the patient. FIG. 7 is a front view of a standing patient 1070 with an intelligent implant, e.g., knee prosthesis 1072 with an IRP 104a, implanted to replace his left knee joint, and of the x-axis 1060 and the y-axis 1062 of the IMU 1022 of the IRP. FIG. 8 is a side view of the patient 1070 of FIG. 7 in a supine position, and of the y-axis 1062 and the z-axis 1064 of the IMU 1022 the IRP, wherein the knee prosthesis 1072 is shown through the patient's right leg.

The IMU 1022 of the IRP 104a includes three accelerometers, each of which senses and measures an acceleration a(g) along a respective one of the x-axis 1060, the y-axis 1062, and the z-axis 1064, where aₓ(g) is the acceleration in units of g-force (g) along the x axis, a_{y}(g) is the acceleration along the y axis, and a_{z}(g) is the acceleration along the z axis. Each accelerometer generates a respective analog sense or output signal having an instantaneous magnitude that represents the instantaneous magnitude of the sensed acceleration along the corresponding axis. For example, the magnitude of the magnitude of the accelerometer output signal at a given time is proportional the magnitude of the acceleration along the accelerometer's sense axis at the same time.

The IMU 1022 also includes three gyroscopes, each of which senses and measures angular velocity Ω(dps) about a respective one of the x-axis 1060, the y-axis 1062, and the z-axis 1064, where Ωₓ(dps) is the angular velocity in units of degrees per second (dps) along the x axis, Ω_{y}(dps) is the angular velocity along the y axis, and Ω_{z}(dps) is the angular velocity along the z axis. Each gyroscope generates a respective analog sense or output signal having an instantaneous magnitude that represents the instantaneous magnitude of the sensed angular velocity about the corresponding axis. For example, the magnitude of the gyroscope output signal at a given time is proportional the magnitude of the angular velocity about the gyroscope's sense axis at the same time.

The IMU 1022 in one embodiment includes at least two analog-to-digital converters (ADCs) for each axis 1060, 1062, and 1064, one ADC for converting the output signal of the corresponding accelerometer into a corresponding digital acceleration signal, and the other ADC for converting the output signal of the corresponding gyroscope into a corresponding digital angular-velocity signal. For example, each of the ADCs may be an 8-bit, 16-bit, or 24-bit ADC.

Each ADC may be configured to have respective parameter values that are the same as, or that are different from, the parameter values of the other ADCs. Examples of such parameters having settable values include sampling rate, dynamic range at the ADC input node(s), and output data rate (ODR). One or more of these parameters may be set to a constant value, while one or more others of these parameters may be settable dynamically (e.g., during run time). For example, the respective sampling rate of each ADC may be settable dynamically so that during one sampling period the sampling rate has one value and during another sampling period the sampling rate has another value.

For each digital acceleration signal and for each digital angular-velocity signal, the IMU 1022 can be configured to provide the parameter values associated with the signal. For example, the IMU 1022 can provide, for each digital acceleration signal and for each digital angular-velocity signal, the sampling rate, the dynamic range, and a time stamp indicating the time at which the first sample or the last sample was taken. The IMU 1022 can be configured to provide these parameter values in the form of a message header (the corresponding samples form the message payload) or in any other suitable form.

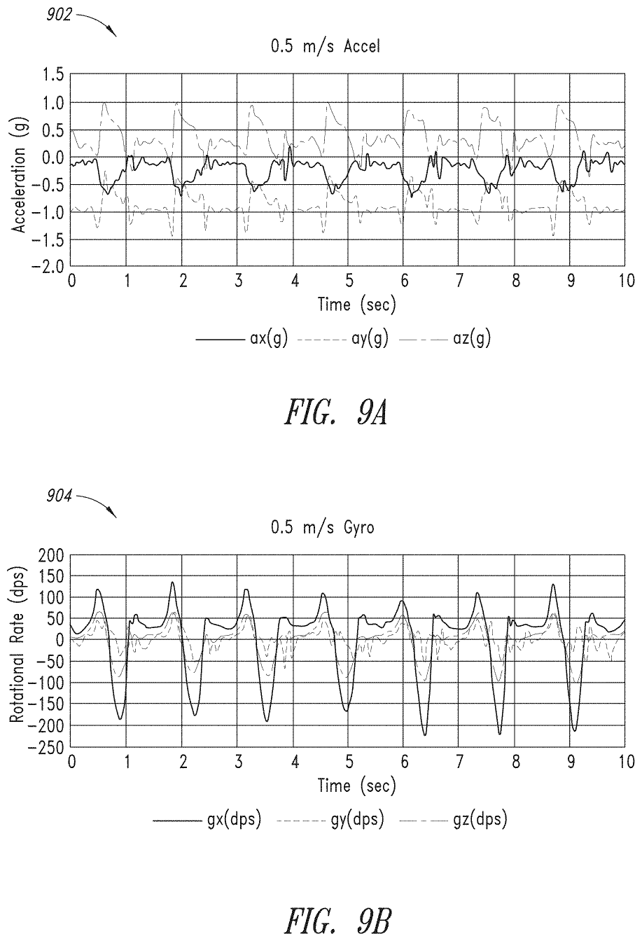

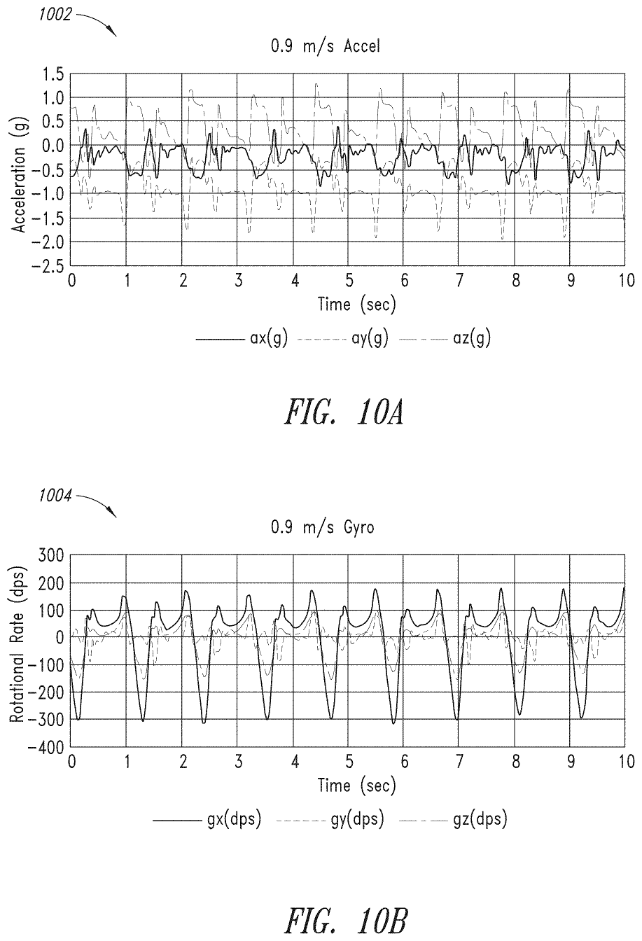

FIG. 9A is a plot 902, versus time, of the digitized versions of the analog acceleration signals aₓ(g), a_{y}(g), and a_{z}(g) as a function of time that the accelerometers of the IMU 1022 respectively generate in response to accelerations along the x axis 1060, the y axis 1062, and the z axis 1064 while the patient 1070 is walking forward with a normal gait at a speed of 0.5 meters/second and for a period of about ten seconds. In this example, the IMU 1022 samples each of the analog acceleration signals aₓ(g), a_{y}(g), and a_{z}(g) at the same sample times, the sampling rate is 3200 Hz, and the output data rate (ODR) is 800 Hz. The ODR is the rate of the samples output by the IMU 1022 and is generated by down sampling the samples taken at 3200 Hz. That is, because 3200 Hz/800 Hz=4, the IMU 1022 generates an 800 Hz ODR by outputting only every fourth sample taken at 3200 Hz.

FIG. 9B is a plot 904, versus time, of the digitized versions of the analog angular-velocity signals Ωₓ(dps), Ω_{y}(dps), and Ω_{z}(dps) (denoted gₓ(dps), g_{y}(dps), and g_{z}(dps), respectively, in FIG. 9B) as a function of time that the gyroscopes of the IMU 1022 respectively generate in response to angular velocities about the x axis 1060, the y axis 1062, and the z axis 1064 while the patient 1070 is walking forward with a normal gait at a speed of 0.5 meters/second and for a period of about ten seconds. In this example, the IMU 1022 samples each of the analog angular-velocity signals Ωₓ(dps), Ω_{y}(dps), and Ω_{z}(dps) and each of the analog acceleration signals aₓ(g), a_{y}(g), and a_{z}(g) at the same sample times and at the same sampling rate of 3200 Hz and ODR of 800 Hz. That is, the plot 904 is aligned, in time, with the plot 902 of FIG. 9A.

FIG. 10A is a plot 1002, versus time, of the digitized versions of the analog acceleration signals aₓ(g), a_{y}(g), and a_{z}(g) as a function of time that the accelerometers of the IMU 1022 respectively generate in response to accelerations along the x axis 1060, the y axis 1062, and the z axis 1064 while the patient 1070 is walking forward with a normal gait at a speed of 0.9 meters/second and for a period of about ten seconds. In this example, the IMU 1022 samples each of the analog acceleration signals aₓ(g), a_{y}(g), and a_{z}(g) at the same sample times, the sampling rate is 3200 Hz, and the output data rate (ODR) is 800 Hz. The ODR is the rate of the samples output by the IMU 1022 and is generated by down sampling the samples taken at 3200 Hz. That is, because 3200 Hz/800 Hz=4, the IMU 1022 generates an 800 Hz ODR by outputting only every fourth sample taken at 3200 Hz.

FIG. 10B is a plot 1004, versus time, of the digitized versions of the analog angular-velocity signals Ωₓ(dps), Ω_{y}(dps), and Ω_{z}(dps) (denoted gₓ(dps), g_{y}(dps), and g_{z}(dps), respectively, in FIG. 10B) as a function of time that the gyroscopes of the IMU 1022 respectively generate in response to angular velocities about the x axis 1060, the y axis 1062, and the z axis 1064 while the patient 1070 is walking forward with a normal gait at a speed of 0.5 meters/second and for a period of about ten seconds. In this example, the IMU 1022 samples each of the analog angular-velocity signals Ωₓ(dps), Ω_{y}(dps), and Ω_{z}(dps) and each of the analog acceleration signals aₓ(g), a_{y}(g), and a_{z}(g) at the same sample times and at the same sampling rate of 3200 Hz and ODR of 800 Hz. That is, the plot 1004 is aligned, in time, with the plot 1002 of FIG. 10A.

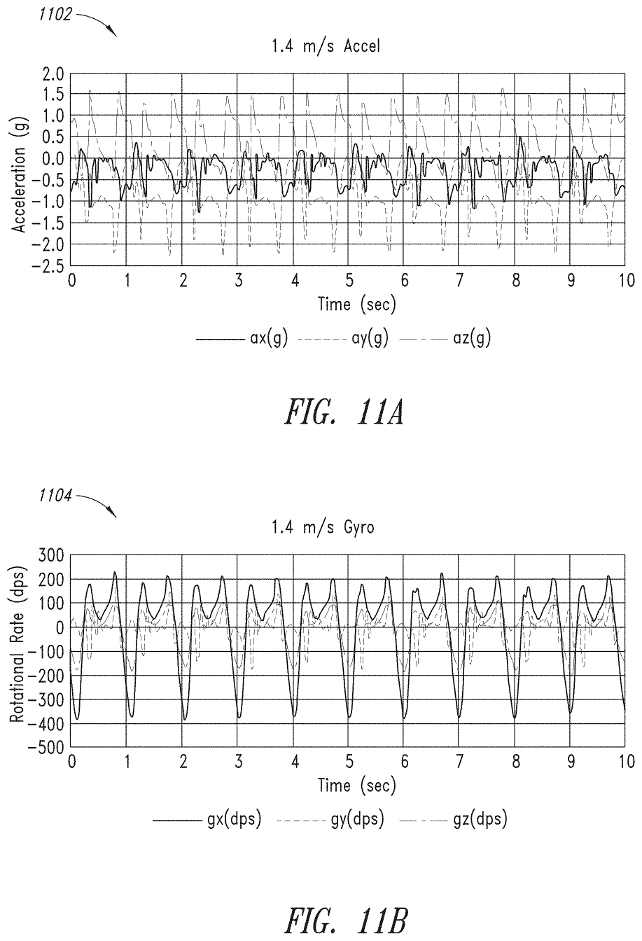

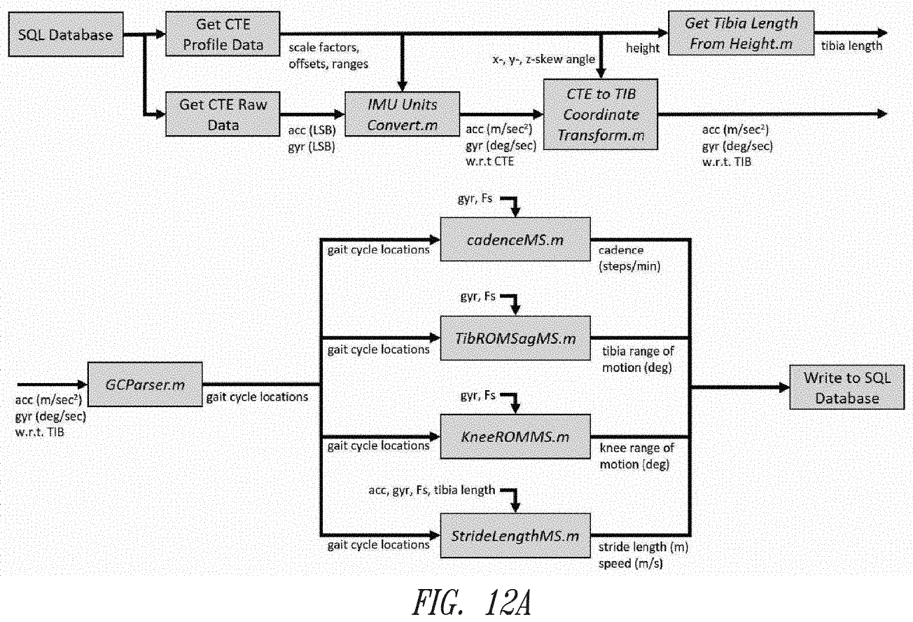

FIG. 11A is a plot 1102, versus time, of the digitized versions of the analog acceleration signals aₓ(g), a_{y}(g), and a_{z}(g) as a function of time that the accelerometers of the IMU 1022 respectively generate in response to accelerations along the x axis 1060, the y axis 1062, and the z axis 1064 while the patient 1070 is walking forward with a normal gait at a speed of 1.4 meters/second and for a period of about ten seconds. In this example, the IMU 1022 samples each of the analog acceleration signals aₓ(g), a_{y}(g), and a_{z}(g) at the same sample times, the sampling rate is 3200 Hz, and the output data rate (ODR) is 800 Hz. The ODR is the rate of the samples output by the IMU 1022 and is generated by down sampling the samples taken at 3200 Hz. That is, because 3200 Hz/800 Hz=4, the IMU 1022 generates an 800 Hz ODR by outputting only every fourth sample taken at 3200 Hz.

FIG. 11B is a plot 1104, versus time, of the digitized versions of the analog angular-velocity signals Ωₓ(dps), Ω_{y}(dps), and Ω_{z}(dps) (denoted gₓ(dps), g_{y}(dps), and g_{z}(dps), respectively, in FIG. 11B) as a function of time that the gyroscopes of the IMU 1022 respectively generate in response to angular velocities about the x axis 1060, the y axis 1062, and the z axis 1064 while the patient 1070 is walking forward with a normal gait at a speed of 0.5 meters/second and for a period of about ten seconds. In this example, the IMU 1022 samples each of the analog angular-velocity signals Ωₓ(dps), Ω_{y}(dps), and Ω_{z}(dps) and each of the analog acceleration signals aₓ(g), a_{y}(g), and a_{z}(g) at the same sample times and at the same sampling rate of 3200 Hz and ODR of 800 Hz. That is, the plot 1104 is aligned, in time, with the plot 1102 of FIG. 11A.

### Gait Parameters

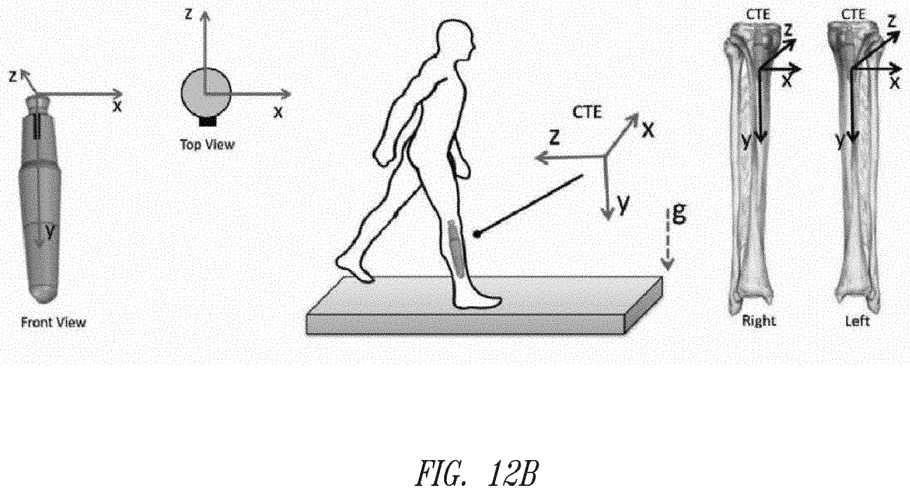

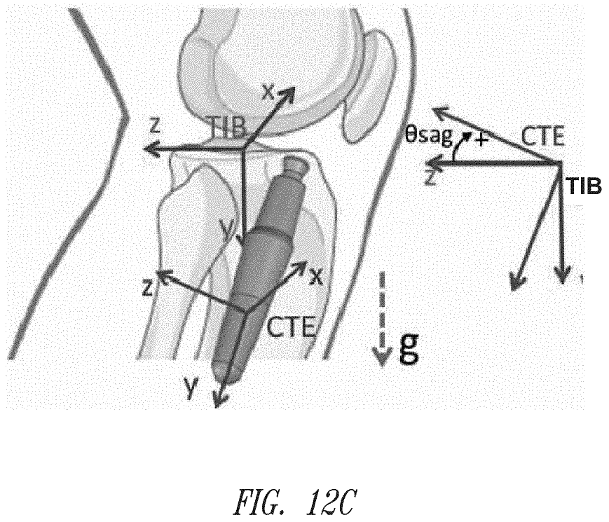

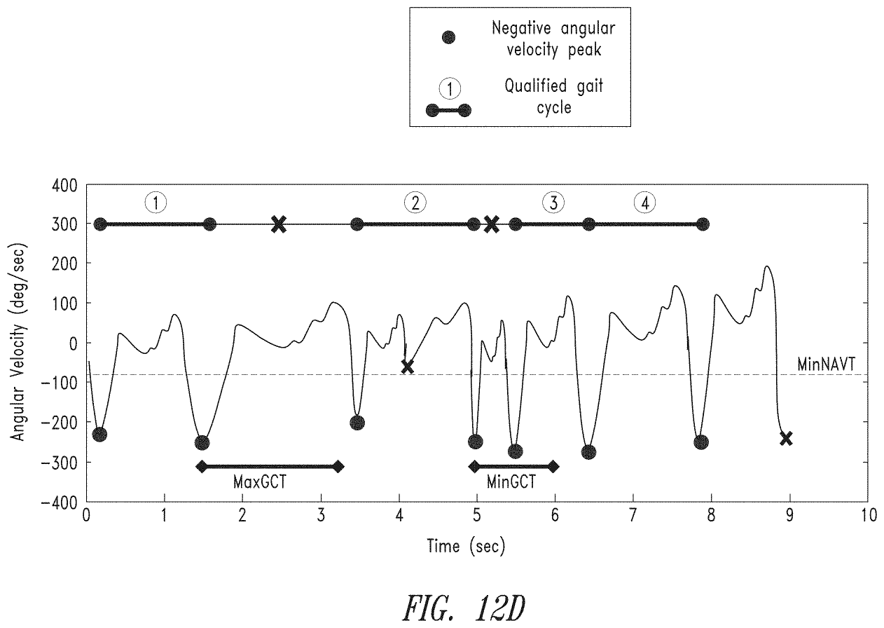

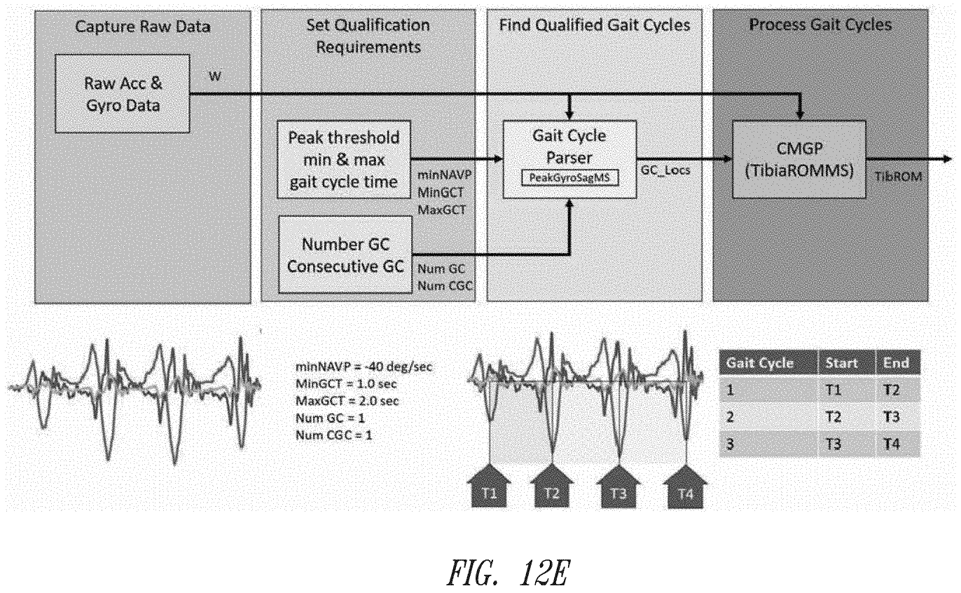

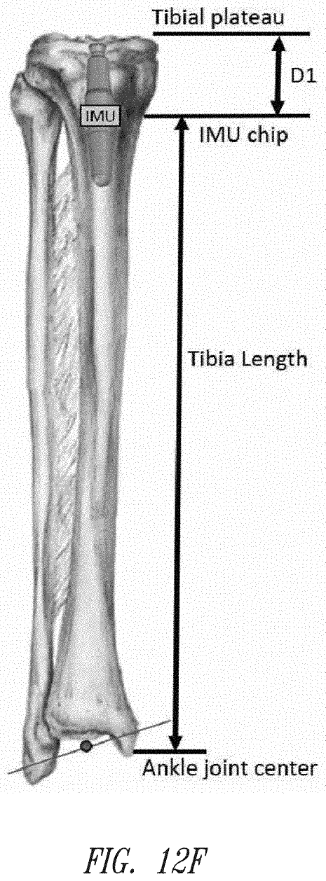

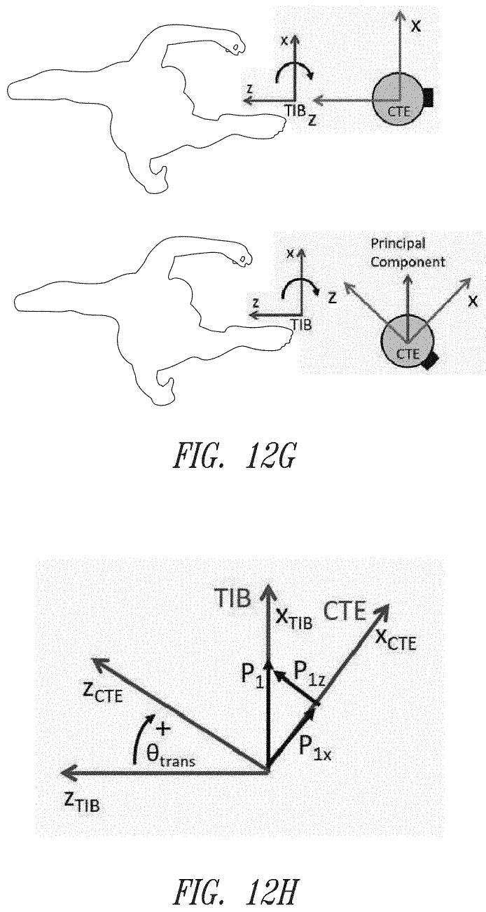

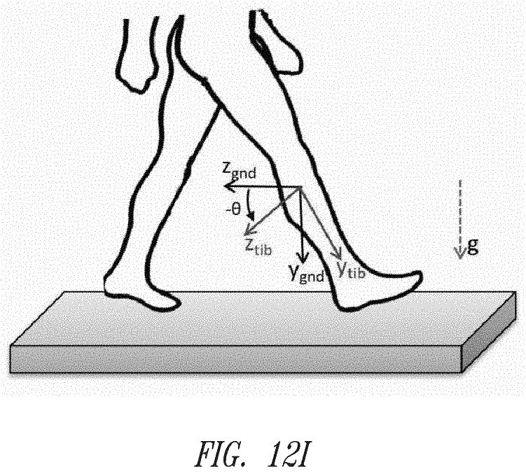

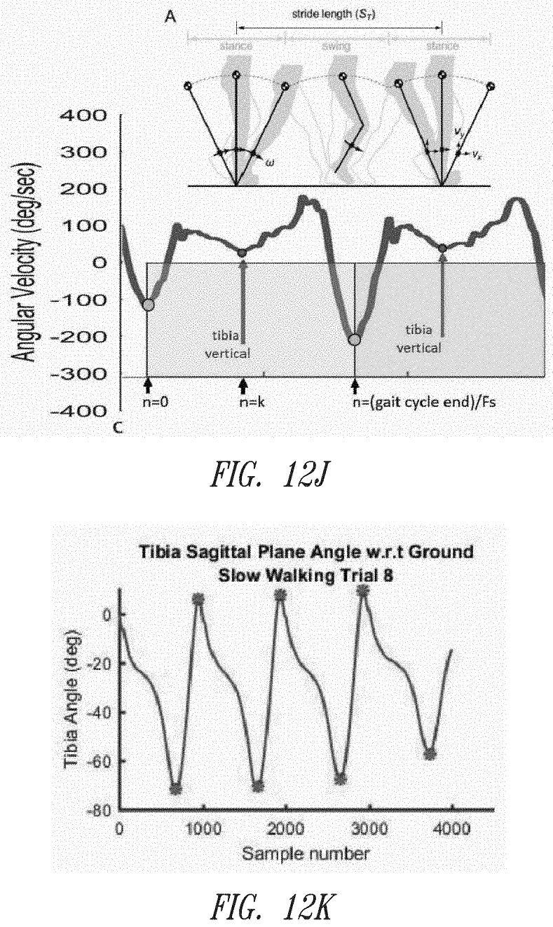

The acceleration signals and angular-velocity signals provided by the IMU 1022 may be processed to detect qualified gait cycles within a bout and to determine kinematic information or kinematic features of the patient based on the qualified gait cycles. For example, the acceleration signals and angular-velocity signals may be processed to determine a set of gait parameters for the bout including: cadence, stride length, walking speed, tibia range of motion, knee range of motion, step count and distance traveled. The step count, distance traveled, cadence, stride length, and walking speed represent measures of activity and robustness of activity. With reference to FIG. 12A, the general programmatic flow to calculate the gait parameters is as follows:

Parameters or calibration values (scale factors, offsets, and ranges) as well as a bout of raw acceleration (LSB) and gyroscope (LSB) data for a subject patient are retrieved. These parameters may be stored in a database. The calibration values (scale factors, offsets, and ranges) are used to convert a bout of raw acceleration and gyroscopic signals into SI units of meters/second and degrees/second, respectively. Transverse plane skew angles (θtrans) are then determined. The acceleration and gyroscopic data are then transformed from an implant coordinate system (sometimes referred to herein as a CTE coordinate system) into a tibia (TIB) coordinate system. A gait cycle parser function operates on the transformed acceleration and gyroscopic signals to identify the temporal start location and end location of qualified gait cycles. Gait cycle start and end locations, sampling frequency (Fs), acceleration and gyroscopic data are then used to calculate the gait parameters. Individual values for these parameters may be based on a single bout, e.g., 10 seconds, of data. Average values of these parameters may be calculated based on bouts of data collected over longer periods of time, e.g., a 24-hour period.

The following sections describe details of the above general process flow of FIG. 12A.

### Implant Coordinate System

Acceleration and gyroscope data are collected with respect to the implant coordinate system. The orientation of the IMU 1022 within the implant establishes the orientation of the implant coordinate system or CTE coordinate system. With reference to FIG. 12B, when implanted into a right tibia the x-axis of the CTE coordinate system points to the left (medially), the y-axis points inferiorly, and the z-axis points posteriorly. When implanted into a left tibia the x-axis points to the left (laterally), the y-axis points inferiorly down the long axis of the tibia, and the z-axis points posteriorly. Continuing with FIG. 12B, the y-axis of the CTE coordinate system points down the long axis of the implant, the z-axis points opposite the black box alignment mark, and the x-axis follows the right-hand rule (left image).

### Tibia Coordinate System

The tibia coordinate system (TIB) is a coordinate system affixed to the tibia with a known constant relationship to the CTE coordinate system. While the CTE coordinate system is defined by the mechanical orientation of the IMU 1022, the TIB coordinate system is expected to be grossly aligned with the anatomical planes of the tibia. The orientation of the implant coordinate system with respect to the TIB coordinate system is defined by the sagittal, frontal, and transverse plane skew angles. Skew angles are used to define the orientation of the CTE coordinate system with respect to the TIB coordinate system. The sagittal plane skew angle rotates the implant about the TIB sagittal plane. The frontal and transverse plane skew angles are similarly defined. With reference to FIG. 12C, the TIB coordinate system is an anatomical coordinate system attached to the tibia. It is expected to be grossly aligned with the anatomical planes of the body. The orientation of the implant with respect to the TIB is defined by the sagittal, frontal, and transverse plane skew angles. In FIG. 12C, the implant is rotated approximately +15 degrees from the TIB coordinate system (θsag=+15°). Positive sagittal plane rotation is defined by right hand rule rotation of the Implant frame about the TIB x-axis.

### Standardized Input and Output Tables

Table 1 defines standard input parameters used to calculate gait parameters. These input parameters are patient specific and may be stored in a database and retrieved at the time of calculating the gait parameters.

TABLE 1

| Parameter | Units | Description |
|---|---|---|
| Height | M | Patient body height during quiet neutral |
| | | upright stance. |
| Skewₜᵣₐₙₛ | Deg | Patient specific transverse plane rotation of |
| | | the implant with respect to the tibia. |
| skeW_{sag} | Deg | Patient specific sagittal plane rotation of the |
| | | implant with respect to the tibia. |
| skew_{front} | Deg | Patient specific frontal plane rotation of the |
| | | implant with respect to the tibia. |

An example of a standard input parameter table with normative values is shown in Table 2.

TABLE 2
Input Parameters

| Height | Skew_{sag} | Skew_{front} | Skewₜᵣₐₙₛ |
|---|---|---|---|
| m | Deg | deg | deg |
| 1.80 | 0 | 0 | 0 |

For each bout of data (^{˜}10 sec) a standardized output table is calculated and stored in the database. The output table contains both intermediate parameters (e.g., qualified gait cycle, gait cycle start, gait cycle end), as well as the gait parameters (e.g., cadence, stride length, walking speed, tibia ROM, estimated knee ROM, step count, distance traveled). Descriptive statistics can then be calculated from these tables to determine means and standard deviations across bouts, days, weeks, months, etc. Additional parameters used by the gait cycle parse are described below. An example standardized output table is shown in Table 3.

TABLE 3

| Intermediate | | | | | | | | | |
|---|---|---|---|---|---|---|---|---|---|
| Parameters | | | Gait Parameters | | | | | | |
| | Gait | Gait | | | | | Estimated | | |
| Qualified | Cycle | Cycle | | Stride | Walking | Tibia | Knee | Step | Distance |
| Gait Cycle | Start | End | Cadence | Length | Speed | ROM | ROM | count | Traveled |

### Qualified Gait Cycles and Associated Parameters

Gait parameters are calculated using qualified gait cycles. A qualified gait cycle (QGC) meets angular velocity and acceleration magnitude requirements, temporal requirements, and requirements on the number of gait cycles per bout of data and their consecutive nature. The definition of a QGC and the parameters used to define QGC are described in Table 4.

TABLE 4

| Parameter | Units | Description |
|---|---|---|
| MinNAVT | deg/sec | Minimum negative angular velocity threshold (MinNAVT). A threshold |
| | | used to define valid negative angular velocity peak (VNAVP). |
| VNAVP | deg/sec | Valid negative angular velocity peak (VNAVP). A negative sagittal plane |
| | | angular velocity local peak that is more negative than MinNAVT. Note |
| | | that tibia angular velocity is negative during swing phase for both a left |
| | | and right legs. |
| MinGCT | sec | Minimum gait cycle time (MinGCT). A gait cycle has at least MinGCT |
| | | second between VNAVP to be declared a gait cycle. Note that (60 |
| | | sec/MinGCT) = maximum cadence (steps/min). For reference, mean |
| | | cadence (SE) in community dwelling older (>60, mean 70.6 years) |
| | | adults was reported as 106.7 steps/min (0.42). |
| MaxGCT | sec | Maximum gait cycle time (MaxGCT). A gait cycle has less than MaxGCT |
| | | second between velocity peaks to be declared a gait cycle. Note that |
| | | (60 sec/MaxGCT) = minimum cadence (steps/min). |
| MaxAcc | m/sec² | Gait cycles with acceleration signals more than MaxAcc m/sec² will not |
| | | be qualified. |
| MaxGyrTrans | deg/sec | Gait cycles with angular velocity in the transverse plane more than |
| | | MaxGyrTrans will not be qualified. |
| MaxGyrFront | deg/sec | Gait cycles with angular velocity in the frontal plane more than |
| | | MaxGyrFront will not be qualified. |
| GC | None | Gait cycle (GC). Two consecutive valid negative tibia angular velocity |
| | | peaks that are temporally separated by more than minimum gait cycle |
| | | time (MinGCT) and less than maximum gait cycle time (MaxGCT). |
| CGC | None | Consecutive gait cycles (CGC). Two gait cycles which share a common |
| | | VNAVP, i.e. the end time of the first gait cycle is identical to the start |
| | | time of the second gait cycle. |
| RGC | None | Required gait cycle (RGC). Bouts of data which have fewer gait cycles |
| | | than the RGC will be considered to have no qualified gait cycles. For |
| | | example, if a bout of data has one GC and the RGC is two, then that GC |
| | | will not be considered a QGC. |
| RCGC | None | Required consecutive gait cycles. Bouts of data which have fewer |
| | | consecutive GCthan RCGC will be considered to have no qualified gait |
| | | cycles. For example, if a bout of data has two consecutive GC and the |
| | | RGC is three, then the two consecutive GC will not be considered QGCs. |
| QGC | None | Qualified gait cycle(s) (QGC). |

Table 5 is a standardized input parameter table with normative values shown.

TABLE 5
Input Parameters

| | | | | | | RGC | RCGC |
|---|---|---|---|---|---|---|---|
| MinNAVT | MinGCT | MaxGCT | MaxAcc | MaxGyrTrans | MaxGyrFront | gait | gait |
| Deg/sec | sec | Sec | m/sec² | deg/sec | deg/sec | cycles | cycles |
| −60 | 1 | 2 | 30 | 320 | 350 | 1 | 1 |

FIG. 12D is a graph showing how qualified gait cycles are identified from a bout of angular velocity data by the gait cycle parser. Local minimum values that are more negative than the minimum negative angular velocity threshold (minNAVT) are shown in large solid dots. Disqualified peaks include the fourth peak because it is more positive than minNAVT and the last peak because it is monotonically decreasing (does not have neighboring data points that are more positive than it). A gait cycle is defined as two negative angular velocity peaks that are temporally separated by more than minimum gait cycle time (MinGCT) and less than maximum gait cycle time (MaxGCT). The time between the second and third negative peak is greater than MaxGCT and thus not a gait cycle, while the fourth and fifth peaks are less than minGCT and thus not a gait cycle. There are four gait cycles in this bout of data. Gait cycle one and two are not consecutive because they do not share a common negative peak, while gait cycle three and four are consecutive.

FIG. 12E is a block diagram showing how qualified gait cycles get parsed from raw IMU data given a set of qualification requirements. In this example, if either the required gait cycle (RGC) or required consecutive gait cycles (RCGC) parameter was set to four then no qualified gait cycles would have been identified because only three gait cycles, which happen to be consecutive, exist in this example bout of data.

The output of the gait cycle parser corresponds to the intermediate parameters listed in Table 3, i.e., the start time and the end time of all the qualified gait cycles in the bout of data. Table 6 below lists the qualification requirements to process each of the gait parameters.

TABLE 6

| Parameter | Data Qualification Requirements |
|---|---|
| Cadence | Calculated from qualified gait cycle(s). |
| Stride length | Calculated from qualified gait cycle(s). |
| Walking speed | Calculated from qualified gait cycle(s). |
| Tibia range of motion | Calculated from qualified gait cycle(s). |
| Knee range of motion | Calculated from qualified gait cycle(s). |
| Step count | None. Commercially available step counter will provide step count. |
| Distance traveled | None. This parameter is derived from step count and mean stride |
| | length. If step count and stride length are qualified this parameter will |
| | be qualified. |

Estimating Tibia Length from Height

Tibia length is used to calculate the stride length and the walking speed gait parameters. With reference to FIG. 12F, tibia length is defined herein as the distance between the ankle joint center and IMU 1022 within the implant. D1 is the distance between the tibial plateau and the IMU 1022 within the implant. The tibia length may be estimated using Equation 1 below:

$$TibiaLength ( m ) = \frac{\frac{( height \times 100 - C 2 )}{C 1} - D 1}{100} \qquad Eq . 1$$

where:
height=the height of the patient,
C1 and C2 are conversion parameters, such as parameters observed in a population; examples of conversion parameters are found in Table 7 (below),
D1=the distance between the ankle joint center and IMU 1022 within the implant

In some embodiments, the conversion parameters may be based on a statistical analysis of tibial length for populations with particular demographic characteristics, such as gender, ethnicity, age, other characteristics, or some combination thereof. Table 7 below provides sample values for conversion parameters based on combinations of values for two different demographic characteristics.

TABLE 7

| Demographic Characteristics | C1 | C2 |
|---|---|---|
| Characteristic 1 = value a; | 2.45 | 75.15 |
| Characteristic 2 = value y | | |
| Characteristic 1 = value b; | 2.60 | 73.23 |
| Characteristic 2 = value y | | |
| Characteristic 1 = value a; | 2.90 | 64.03 |
| Characteristic 2 = value z | | |
| Characteristic 1 = value b; | 2.79 | 70.81 |
| Characteristic 2 = value z | | |
| Mean | 2.685 | 70.805 |

Converting Acceleration and Gyroscope Data from LSB to Meters/Second and Degrees/Second

The parameters defined in Table 8 are retrieved from the database and used in the conversion calculation.

TABLE 8

| Parameter | Nominal | Units | Description |
|---|---|---|---|
| acx_sf | 1.000 | NA | Accelerometer scale factor for the x-, y-, and z-axis as determined |
| acy_sf | | | by the final functional test calibration procedure. Once the implant |
| acz_sf | | | (CTE) is manufactured and calibrated this value is not expected to |
| | | | change. The units are dimensionless. This parameter is stored in |
| | | | the cte_profile table in the database. |
| acx_ofs | 0.000 | m/sec² | Accelerometer offset for the x-, y-, and z-axis determined by the |
| acy_ofs | | | CTE final functional test calibration procedure. Once the CTE is |
| acz_ofs | | | manufactured and calibrated this value is not expected to change. |
| | | | The units are (m/sec²). This parameter is stored in the cte_profile |
| | | | table in the database. |
| AC_RG | 8.000 | g | Accelerometer range. This value is set by the CTE firmware and |
| | | | stored in the cte_dataheader table in the database. Each recorded |
| | | | bout of data has an accelerometer range associated with it. |
| ACU | $\frac{9.8066}{{2}^{15}}$ | $\frac{m}{{s}^{2} \cdot g \cdot LSB}$ | Constant converting binary data and g-based IMU range setting to m/sec². ACU has the constant value of 9.8066/2\hat{ }15. |
| rrx_sf | 1.000 | 1/LSB | Gyroscope scale factor for the x-, y-, and z-axis as determined by |
| rry_sf | | | the final functional test calibration procedure. Once the CTE is |
| rrz_sf | | | manufactured and calibrated this value is not expected to change. |
| | | | The units are (1/LSB). This parameter is stored in the cte_profile |
| | | | table in the database. |
| rrx_ofs | 0.000 | deg/sec | Gyroscope offset for the x-, y-, and z-axis determined by the CTE |
| rry_ofs | | | final functional test calibration procedure. Once the CTE is |
| rrz_ofs | | | manufactured and calibrated this value is not expected to change. |
| | | | The units are (deg/sec). This parameter is stored in the cte_profile |
| | | | table in the database. |
| RR_RG | 1000 | deg/sec | Gyroscope range. This value is set by the CTE firmware and stored |
| | | | in the cte_dataheader table in the database. Each recorded bout |
| | | | of data has a gyroscope range associated with it. |
| RRU | $\frac{1}{{2}^{15}}$ | 1/LSB | Constant used to convert binary (LSB) data to a unitless value which is multiplied by RR_RG to get units of deg/sec. RRU has the |
| | | | constant value of 1/2¹⁵. |

The raw acceleration and gyroscope data are converted from least significant bits (LSB) to SI units of m/sec² and deg/sec using the parameters of Table 8 and following equations:

acx=AC_RG·ACU·[acx_sf·Aₓ]−acx_ofs Eq. 2

rrx=RR_RG·RRU·[rrx_sf·Rₓ]−rrx_ofs Eq. 3

Where Aₓ(LSB) and Rₓ(LSB) are defined as the recorded x-axis acceleration and angular velocity signals in LSB, respectively. These equations are repeated for the y- and z-axis, using the appropriate y- and z-axis parameters to determine the acceleration and angular velocity in (m/s²) and (deg/s) for all three axes.

Transposing Data from Implant Coordinate System to Tibia Coordinate System

Data recorded in the implant (CTE) coordinate system is transformed into data with respect to the tibia (TIB) coordinate system. To this end, with reference to FIG. 12C, the CTE acceleration and angular velocity data are multiplied by a rotation matrix to transform it into the TIB coordinate system. The CTE is described with respect to the TIB coordinate system using fixed angle rotations in the order of transverse (y-axis), sagittal (x-axis), and frontal (z-axis). With this convention, the orientation of the implant (CTE) with respect to the TIB is defined as follows:

Start with the CTE coincident with TIB.

Rotate the CTE about the y-axis of the TIB by amount skewₜᵣₐₙₛ.

Rotate the CTE about the x-axis of the TIB by amount skew_{sag}.

Rotate the CTE about the z-axis of the TIB by amount skew_{front}.

Following this convention, the rotation matrix R_{TIB_CTE} may be mathematically defined to be a matrix that transforms data from the CTE coordinate system into the TIB coordinate system.

R_{TIB_CTE}=R_{Z}(skew_{front})Rₓ(skew_{sag})R_{y}(skewₜᵣₐₙₛ) Eq. 4

Using the definition of elemental rotations about the y-, x-, and z-axes by amounts skewₜᵣₐₙₛ, skew_{sag}, and skew_{front} the following results:

$${R}_{TIB \_ CTE} = [ \begin{matrix}{C}_{front} & - {S}_{front} & 0 \\ {S}_{front} & {C}_{front} & 0 \\ 0 & 0 & 1\end{matrix} ] [ \begin{matrix}1 & 0 & 0 \\ 0 & {C}_{sag} & - {S}_{sag} \\ 0 & {S}_{sag} & {C}_{sag}\end{matrix} ] [ \begin{matrix}{C}_{trans} & 0 & {S}_{trans} \\ 0 & 1 & 0 \\ - {S}_{trans} & 0 & {C}_{trans}\end{matrix} ] \qquad Eq . 5$$

Where C_{front} and S_{front} are shorthand for cosine(skew_{front}) and sine(skew_{front}) respectively. Cosine and sine function operating on the skew_{sag} and skewₜᵣₐₙₛ angles are similarly defined.

Acceleration and angular velocity data can then be transformed from the implant coordinate system to the TIB coordinate using the following equations.

$${[ \begin{matrix}{a}_{x} \\ {a}_{y} \\ {a}_{z}\end{matrix} ]}^{TIB} = {R}_{TIB \_ CTE} \times {[ \begin{matrix}{a}_{x} \\ {a}_{y} \\ {a}_{z}\end{matrix} ]}^{CTE} where : \qquad Eq . 6$$

$${[ \begin{matrix}{a}_{x} \\ {a}_{y} \\ {a}_{z}\end{matrix} ]}^{TIB} is the acceleration with respect to the TIB coordinate system , and$$

$${[ \begin{matrix}{a}_{x} \\ {a}_{y} \\ {a}_{z}\end{matrix} ]}^{CTE} is the acceleration with respect to the CTE coordinate system .$$

$${[ \begin{matrix}{w}_{x} \\ {w}_{y} \\ {w}_{z}\end{matrix} ]}^{TIB} = {R}_{TIB \_ CTE} \times {[ \begin{matrix}{w}_{x} \\ {w}_{y} \\ {w}_{z}\end{matrix} ]}^{CTE} \\ where : \qquad Eq . 7$$

$${[ \begin{matrix}{w}_{x} \\ {w}_{y} \\ {w}_{z}\end{matrix} ]}^{TIB} is the angular velocity with respect to the TIB coordinate system ,$$

$${[ \begin{matrix}{w}_{x} \\ {w}_{y} \\ {w}_{z}\end{matrix} ]}^{CTE} is the angular velocity with respect to the CTE coordinate system .$$

Equation Eq. 6 and Eq. 7 are used to transform data collected in the CTE coordinate system into the TIB coordinate system.

Following CTE implant, the relationship between the implant (CTE) coordinate system and the TIB coordinate system is fixed and constant for that patient. As such, the parameters skew_{sag}, skewₜᵣₐₙₛ, and skew_{front}, which define this relationship are constant, and in some embodiments may be stored as the patient's standard input parameters (see Table 1) in the database.

### Calculating Skew Angles

The skew angle values for an implant may be determined according to expert knowledge or via a dynamic calibration function.

Regarding expert knowledge, the skew angles can be specified according to expert knowledge of the orientation of the CTE implant with respect to the tibia long axis. For example, the sagittal plane skew angle may be set to 5° and the frontal and transverse plane skew angles set to zero per an understanding of the typical CTE alignment following surgical implantation.

If the skew angles are all set to zero than the TIB coordinate system and the CTE coordinate system are identical and a CTE to TIB coordinate function applies a unity transformation (identity matrix multiplication) to the acceleration and gyroscope data. The acceleration and angular velocity data is still expressed in terms of the CTE coordinate system, and the gait parameters are calculated based on the non-transformed IMU data. In one example, the sagittal plane skew angle was manually set to 5° and the transverse and frontal plane skew angles were set to zero based on the presumed orientation of the CTE with respect to the tibia.

Regarding the dynamic calibration function, this function returns a transverse plane skew angle defining a sagittal plane which captures most of the angular velocity signal for that bout of data.

The transverse plane skew angle can be calculated from any walking data using the dynamic calibration function. With reference to FIG. 12G, which shows the transverse plane, this function utilizes principal component analysis to determine the plane, with respect to the implant (CTE) coordinate system which captures, in a least squares sense, the majority of the angular velocity signal. For example, assuming the patient walks such that the majority of his leg swing (IMU angular velocity) is about the CTE coordinate system x-axis. In this scenario the dynamic calibration function is configured to return a zero value (or a value within a threshold range of zero) for the transverse plane skew angle because most of the angular velocity is occurring about the CTE x-axis. A transverse plane skew angle of 0° means the TIB y-z plane is parallel to the CTE y-z plane. In a second hypothetical situation, assuming the patient swings their leg about an axis that is rotated 450 in the CTE transverse plane (right hand rule positive rotation about the CTE y-axis). In this scenario the principal component of the measured signal with respect to the CTE points in the positive CTE x- and z-axis direction. And the dynamic calibration function is configured to return a value of 45° (or a value within a threshold range of 450).

The transverse plane skew angle is calculated as follows.

1. The first principal component (P₁) of the angular velocity time series matrix (W) is calculated.

$${P}_{1} = \begin{matrix}{P}_{x} \\ {P}_{y} = P C A ( W ) \\ {P}_{z}\end{matrix} \qquad Eq . 8$$

2. If the transverse plane skew angle is positive (the implant (CTE) is externally rotated from the TIB coordinate system) for a right leg, P1 is expected to have positive y- and z-axis components.

3. With reference to FIG. 12H, the transverse plane angular rotation of the principal component with respect to the CTE coordinate system is given by the four-quadrant inverse tangent of the z-axis and y-axis components of P1, in degrees (Eq. 9Eq.).

θₜᵣₐₙₛ=atan 2d(P_{1z},P₁ₓ) Eq. 9

The trigonometric diagram of FIG. 12H shows how the transverse plane skew angle is calculated from the first principal component (P1) of the angular velocity matrix (W). Shown here is the CTE coordinate system (CTE) with the first principal component of the angular velocity matrix (P1) shown with a positive transverse plane angular rotation of (θₜᵣₐₙₛ). θₜᵣₐₙₛ is given by the four quadrant inverse tangent of P1z and P1x.

### Step Count

Step count corresponds to the accumulated number of steps (e.g., detected during a bout). The IMU 1022 is configured to provide a step count in accordance with commercially available step counters, such as is included in the Bosch BMI 160 inertial measurement unit.

### Cadence

Cadence may be provided as the average walking step rate measured as steps per minute, using the following equation. Note that there are two steps per gait cycle.

$$cadence ( \frac{step s}{min} ) = 2 \frac{s t e p s}{gait cycle} \cdot \frac{1 ( gait cycle )}{( GC End Time ) - ( GC Start Time ) ( sec )} \cdot \frac{60 ( s e c )}{1 ( min )} \qquad Eq . 10$$

where:

GC End Time and GC Start Time for the relevant gait cycle are found in Table 3.

### Stride Length, Walking Speed, and Distance Traveled

FIG. 12I illustrates the coordinate system of the tibia (tib) and ground (gnd) when walking. Positive rotation of the tibia follows the right-hand rule with the x-axis pointing medially. FIG. 12J is a graph of angular velocity of the tibia in the sagittal plane. Thinking of the leg as an inverted pendulum, the shank reaches a local minimum angular velocity when the tibia is approximately vertical. The gait cycle begins at discrete time n=0, coincident with the first valid negative angular velocity peaks (VNAVP) (first big dot). The midpoint between two angular velocity peaks are marked by smaller dots, at discrete time step k, indicating when the tibia is assumed to be vertical

Now, with reference to FIGS. 12I and 12J, average stride length (measured in meters) and average walking speed (measures in meters/second) may be derived from a bout of acceleration and velocity data as follows:

1. Let n be the discrete time variable. Let the gait cycle start and end time be defined by the sagittal plane angular velocity peaks, thus a negative angular velocity peak exists at n=0, by definition.

2. Assume that the tibia is vertical (aligned with the ground y-axis) at the midpoint of the gait cycle, call this discrete time k. Thus, theta is equal to zero at n=k.

θ_{sag}(n=k)=0 deg Eq. 11.1

θ_{front}(n=k)=0 deg Eq. 11.2

θₜᵣₐₙₛ(n=k)=0 deg Eq. 11.3

3. The tibia angular displacement with respect to time can be expressed as the discrete time integral of the angular velocity over one gait cycle.

θ_{sag}(n)=Σ₀ⁿw_{sag}(n)dτ+θ_{sag}(0) Eq. 12.1

θ_{front}(n)=Σ₀ⁿw_{front}(n)dτ+θ_{front}(0) Eq. 12.1

θₜᵣₐₙₛ(n)=Σ₀ⁿwₜᵣₐₙₛ(n)dτ+θₜᵣₐₙₛ(0) Eq. 12.1

4. Evaluate respective Eqs. 12 at n=k and use corresponding Eqs. 11 to solve for the initial condition θ(0).

θ_{sag}(n=k)=Σ0=Σ₀^{k}w_{sag}(n)dτ+θ_{sag}(0) Eq. 13.1

θ_{front}(n=k)=0=Σ₀^{k}w_{front}(n)dτ+θ_{sag}(0) Eq. 13.2

θₜᵣₐₙₛ(n=k)=0=Σ₀^{k}wₜᵣₐₙₛ(n)dτ+θ_{sag}(0) Eq. 13.3

−Σ₀^{k}w_{sag}(n)dτ=θ_{sag}(0) Eq. 14.1

−Σ₀^{k}w_{front}(n)dτ=θ_{front}(0) Eq. 14.2

−Σ₀^{k}wₜᵣₐₙₛ(n)dτ=θₜᵣₐₙₛ(0) Eq. 14.3

5. Use the respective initial condition θ(0) to calculate the tibia angular displacement with respect to time.

θ_{sag}(n)=Σ₀ⁿw_{sag}(n)dτ+θ_{sag}(0) Eq. 15.1

θ_{front}(n)=Σ₀ⁿw_{front}(n)dτ+θ_{front}(0) Eq. 15.2

θₜᵣₐₙₛ(n)=Σ₀ⁿwₜᵣₐₙₛ(n)dτ+θₜᵣₐₙₛ(0) Eq. 15.3

6. Transform the accelerations measured by the IMU 1022 in the tibia (tib) reference frame to the ground (GND) reference frame given the angular displacement of the IMU 6(n). Subtract gravity from the signal to get the acceleration of the IMU in the GND frame.

$${R}_{G NDTIB} = {R}_{front} {R}_{sag} {R}_{trans} \qquad Eq . 16$$

$$[ \begin{matrix}{a}_{{x}_{g n d}} ( n ) \\ {a}_{{y}_{g n d}} ( n ) \\ {a}_{{z}_{g n d}} ( n )\end{matrix} ] = {R}_{G NDTIB} [ \begin{matrix}{a}_{{x}_{tib}} ( n ) \\ {a}_{{y}_{tib}} ( n ) \\ {a}_{{z}_{tib}} ( n )\end{matrix} ] - [ \begin{matrix}g \\ 0 \\ 0\end{matrix} ] \qquad Eq . 16.2$$

$$[ \begin{matrix}{a}_{z \_ gnd} ( n ) \\ {a}_{x \_ gnd} ( n )\end{matrix} ] = [ \begin{matrix}cos ( θ ( n ) ) & - sin ( θ ( n ) ) \\ sin ( θ ( n ) ) & cos ( θ ( n ) )\end{matrix} ] [ \begin{matrix}{a}_{z \_ tib} ( n ) \\ {a}_{x \_ tib} ( n )\end{matrix} ] - [ \begin{matrix}0 \\ g\end{matrix} ] \qquad Eq . 16.3$$

7. Based on the methods proposed in S. Yang, J. T. Zhang, A. C. Novak, B. Brouwer, and Q. Li, “Estimation of spatio-temporal parameters for post-stroke hemiparetic gait using inertial sensors,” Gait Posture, vol. 37, no. 3, pp. 354-358, March 2013, use knowledge of the angular velocity at time n=k to calculate the linear anterior/posterior (ap) and medial/lateral (ml) velocity at time n=k. Assume the superior/inferior (si) velocity is zero at time n=k.

$${v}_{ap \_ gnd} ( k ) = - {w}_{sag} ( k ) \frac{deg}{s e c} \cdot \frac{2 π L ( m )}{360 ( deg )} \qquad Eq . 17.1$$

$${v}_{ml \_ gnd} ( k ) = - {w}_{front} ( k ) \frac{deg}{s e c} \cdot \frac{2 π L ( m )}{3 \; 60 ( deg )} \qquad Eq . 17.2$$

$${v}_{si \_ gnd} ( k ) = 0 \qquad Eq . 17.3$$

8. The velocity with respect to GND can be written as the discrete time integral of the acceleration with respect to the GND frame.

v_{gnd}(n)=Σ₀^{T}a_{gnd}(n)dτ+v_{gnd}(0) Eq. 19

9. Evaluate Eq. 19 at n=k and use one or more of Eqs. 17 to solve for the initial condition v_{gnd}(0).

$${v}_{ap \_ gnd} ( n = k ) = - {w}_{sag} ( k ) \frac{d e g}{s e c} \cdot \frac{2 π L ( m )}{360 ( d e g )} = {\sum }_{0}^{k} {a}_{ap gnd} ( n ) d τ + {v}_{ap \_ gnd} ( 0 ) \qquad Eq . 20.1$$

$${v}_{ml \_ gnd} ( n = k ) = - {w}_{front} ( k ) \frac{d e g}{s e c} \cdot \frac{2 π L ( m )}{360 ( d e g )} = {\sum }_{0}^{k} {a}_{ml gnd} ( n ) d τ + {v}_{ml \_ gnd} ( 0 ) \qquad Eq . 20.2$$

$${v}_{si \_ gnd} ( n = k ) = 0 = {\sum }_{0}^{k} {a}_{ap gnd} ( n ) d τ + {v}_{si \_ gnd} ( 0 ) \qquad Eq . 20.3$$

$${v}_{ap \_ gnd} ( 0 ) = - {w}_{sag} ( k ) \frac{d e g}{s e c} \cdot \frac{2 π L ( m )}{360 ( d e g )} - {\sum }_{0}^{k} {a}_{ap \_ gnd} ( n ) d τ \qquad Eq . 21.1$$

$${v}_{ml \_ gnd} ( 0 ) = - {w}_{front} ( k ) \frac{deg}{s e c} \cdot \frac{2 π L ( m )}{360 ( deg )} - {\sum }_{0}^{k} {a}_{ml \_ gnd} ( n ) d τ \qquad Eq . 21.2$$

$${v}_{si \_ gnd} ( 0 ) = - {\sum }_{0}^{k} {a}_{si \_ gnd} ( n ) d τ \qquad Eq . 21.3$$

10. Use the initial condition v_{gnd}(0), given by a respective one of Eqs. 20, to solve for the velocity with respect to GND frame by integrating the acceleration.

v_{ap_gnd}(n)=Σ₀^{T}a_{ap_gnd}(n)dτ+v_{ap_gnd}(0) Eq. 22.1

v_{ml_gnd}(n)=Σ₀^{T}a_{mi_gnd}(n)dτ+v_{ml_gnd}(0) Eq. 22.2

v_{si_gnd}(n)=Σ₀^{T}a_{si_gnd}(n)dτ+v_{si_gnd}(0) Eq. 22.3

11. Report walking speed as the 3D magnitude of the mean velocity during this gait cycle.

$$speed = \sqrt{mean {( {v}_{ap \_ gnd} )}^{2} + mean {( {v}_{ml \_ gnd} )}^{2} + mean {( {v}_{si \_ gnd} )}^{2}} \qquad Eq . 23$$

12. Integrate the velocity with respect to the GND frame to calculate the anterior/posterior and superior/inferior position with respect to the GND frame. Ignore medial/lateral translation of the implant (CTE) for step length.

sₐₚ(n)=Σ₀^{T}v_{ap_gnd}(n)dτ Eq. 24.1

sₛᵢ(n)=Σ₀^{T}v_{si_gnd}(n)dτ Eq. 24.2

13. Calculate stride length as the total anterior and superior/inferior distance traveled by the IMU 1022 in the GND frame.

Stride_Length=\sqrt{sₐₚ²(T)+sₛᵢ²(T)} Eq. 25

14. Calculate distance traveled by multiplying total steps by mean stride length.

distance=(step count)·(s̅t̅r̅i̅d̅e̅_̅l̅e̅n̅g̅t̅h̅) Eq. 26

The foregoing process of calculating average stride length and average walking speed is based on techniques disclosed in Q. Li, M. Young, V. Naing, and J. M. Donelan, “Walking speed estimation using a shank-mounted inertial measurement unit,” J. Biomech., vol. 43, no. 8, pp. 1640-1643, May 2010; and L. Wang, Y. Sun, Q. Li, and T. Liu, “Estimation of Step Length and Gait Asymmetry Using Wearable Inertial Sensors,” IEEE Sens. J., vol. 18, no. 9, pp. 3844-3851, May 2018. Average distance traveled (measured in meters) may be derived by multiplying the step count by the average stride length.

### Tibia Range of Motion

The range of motion for the tibia (ROM_{tibia}), calculated from gyroscopic data, represents the angular displacement (arc) of the tibia relative to the ground in the sagittal plane. Simplistically, this can be thought of as the inclusive arc of a pendulum that is translating in the sagittal plane. The tibia ROM is measured based on kinematic data obtained by a sensor while the person is walking, and may be referred to as functional tibia ROM. The tibia ROM may be calculated using the following equation:

$${θ}_{{tibia}_{sag}} ( n ) = {\sum }_{n = 1}^{N} {w}_{sag} ( n ) \cdot T \qquad Eq . 27$$

where:

T is the discrete time sample period (sec.),

n is the discrete time sample number,

N is the total number of samples in the bout of data,

w_{sag} is the angular velocity of the tibia in the sagittal plan, and

θ_{tibia}_{_{sag}} is the angle of the tibia with respect to the floor discrete time signal.

The peaks and valleys of θ_{tibia}_{_{sag}}(n) can be found using peak detection.

$${θ}_{{tibia}_{{sag}_{peaks}}} = {Local Peaks}_{n} [ {θ}_{{tibia}_{sag}} ( n ) ] \qquad Eq . 28$$

$${θ}_{{tibia}_{{sag}_{valleys}}} = 1 \cdot {Local Peaks}_{n} [ - 1 \cdot {θ}_{{tibia}_{sag}} ( n ) ] \qquad Eq . 29$$

With reference to FIG. 12K, the range of motion (ROM) of the tibia in the sagittal plane is defined as the difference between the peaks and valleys of Ø_{tibia}_{_{sag}}.

$${ROM}_{t {ibia}_{sag}} = {θ}_{{tibia}_{{sag}_{peaks}}} - {θ}_{{tibia}_{{sag}_{valleys}}} \qquad Eq . 30$$

It is noted that this is tibia range of motion with respect to the ground, not the femur. Therefore, both hip flexion/extension and knee flexion/extension will influence the tibia range of motion when walking.

### Knee Range of Motion

The IMU measures the motion of the tibia but not the femur. Both hip and knee joint flexion/extension contribute to the angular velocity of the IMU. To estimate the knee joint range of motion, the population mean sagittal plane hip kinematics is added to the tibia sagittal plane kinematics. The knee joint range of motion is calculated assuming “normal” hip joint kinematics as described in D. Winter, The biomechanics and motor control of human gait. Waterloo Ont.: University of Waterloo Press, 1987, in Table 3.32(b).

ϑₕᵢₚ(n) is the population mean “normal” hip kinematics normalized to the gait cycle. Assume peak tibia angular velocity occurs at 87% of gait cycle, as described in FIG. 2 of E. Bishop and Q. Li, “Walking speed estimation using shank-mounted accelerometers,” in 2010 IEEE International Conference on Robotics and Automation, 2010, pp. 5096-5101. To align hip kinematics with gait cycle start (peak tibia angular velocity) circularly shift hip kinematic curve by 87%. The first point of ϑₕᵢₚ(n) is equal to the Winter's normal hip kinematics at 87% of the gait cycle. ϑₕᵢₚ(n) is then resampled to have the same number of data points as w the angular velocity of the tibia in the sagittal plane.

Assume the tibia is vertical at the midpoint of the gait cycle, the angular position of the tibia with respect to the ground, θ_{tibia}_{_{sag}}(n), may be calculated as follows:

$${θ}_{{tibia}_{sag}} ( n ) = {\sum }_{n = 1}^{n} {w}_{sag} ( n ) \cdot T - {\sum }_{n = 1}^{k} {w}_{sag} ( n ) \cdot T \qquad Eq . 31$$

where:
k denotes the discrete time at this point (n=k)
w_{sag} is the angular velocity of the tibia in the sagittal plane,
T is the discrete time sample period (sec.) and
n is the discrete time sample number.

The sagittal plane knee joint angular position (θₖₙₑₑ_{_{sag}}) is estimated as follows:

θₖₙₑₑ_{_{sag}}(n)=[θ_{tibia}_{_{sag}}(n)+ϑₕᵢₚ(n)] Eq. 32

Positive θₖₙₑₑ, and ϑₕᵢₚ are defined as knee flexion and hip flexion, respectively. Via the right-hand rule, positive sensor (IMU) rotation causes knee flexion. Therefore, tibia rotation is added to hip flexion to get knee flexion.

The sagittal plane knee range of motion within one gait cycle (ROMₖₙₑₑ_{_{sag}}) is then be calculated as follows:

$${ROM}_{k n e {e}_{sag}} = {max}_{n} [ {θ}_{k n e {e}_{sag}} ( n ) ] - {min}_{n} [ {θ}_{k n e {e}_{sag}} ( n ) ] \qquad Eq . 33$$

where:
max and min are maximum and minimum operators.

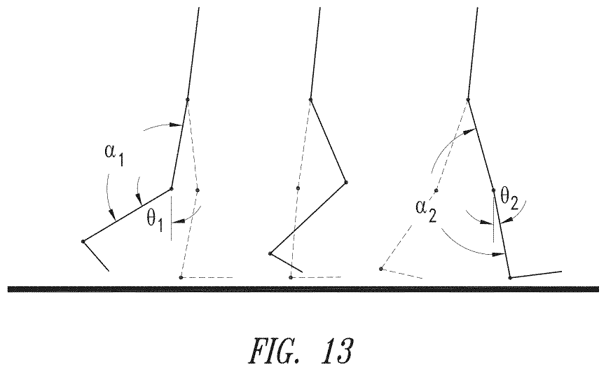

The disclosed estimated range of motion for the knee (ROMₖₙₑₑ) represents the difference between maximum flexion and extension during the gait cycle. In clinical terms, it is a measure of how many degrees a person bends their knee when walking. This calculation is based on a combination of published tabular data for hip angular position (optionally stratified for sex, age, and BMI) combined with the implant's ROM_{tibia} data. This value has the same meaning as the standard of care, clinician static, goniometer measurement taken during a physical exam. However, it represents the actual dynamic range of motion during normal weight-bearing activity as opposed to a static, full capability, range of motion assessed during the physical exam. Knee ROM can be measured based on kinematic data obtained by a sensor while the person is either on a table in a clinical setting (in which case the knee is being bent by a physician) or while the person is walking. If it is based on kinematic data obtained while the person is walking, then it may be referred to as functional knee ROM. Both range of motion measurements are schematically illustrated in FIG. 13 for a right leg motion through one gait cycle, where the angle α is the angle calculated based on a combination of tabular hip and femur reference data and the ROM_{tibia} data and ROM_{tibia}=θ₁+θ₂ and ROMₖₙₑₑ=α₂−α₁.

### Operation of Intelligent Implant

Returning to FIG. 5, operation of an intelligent implant with the implantable reporting processor 1003 is now described. The fuse 1014, which is normally electrical closed, is configured to open electrically in response to an event that can injure the patient in which the IRP 1003 resides, or damage the battery 1012 of the IRP if the event persists for more than a safe length of time. An event in response to which the fuse 1014 can open electrically includes an overcurrent condition, an overvoltage condition, an overtemperature condition, an over-current-time condition, and over-voltage-time condition, and an over-temperature-time condition. An overcurrent condition occurs in response to a current through the fuse 1014 exceeding an overcurrent threshold. Likewise, an overvoltage condition occurs in response to a voltage across the fuse 1014 exceeding an overvoltage threshold, and an overtemperature condition occurs in response to a temperature of the fuse exceeding a temperature threshold. An over-current-time condition occurs in response to an integration of a current through the fuse 1014 over a measurement time window (e.g., ten seconds) exceeding a current-time threshold, where the window can “slide” forward in time such that the window always extends from the present time back the length, in units of time, of the window. Alternatively, an over-current-time condition occurs if the current through the fuse 1014 exceeds an overcurrent threshold for more than a threshold time.

Similarly, an over-voltage-time condition occurs in response to an integration of a voltage across the fuse 1014 over a measurement time window, and an over-temperature-time condition occurs in response to an integration of a temperature of the fuse over a measurement time window. Alternatively, an over-voltage-time condition occurs if the voltage across the fuse 1014 exceeds an overvoltage threshold for more than a threshold time, and an over-temperature-time condition occurs if a temperature associated with the fuse 1014, battery 1012, or electronics assembly 1010 exceeds an overtemperature threshold for more than a threshold time. But even if the fuse 1014 opens, thus uncoupling power from the electronics assembly 1010, the mechanical and structural components of the intelligent implant (not shown in FIG. 5) with which the IRP 1003 is associated are still fully operational. For example, if the intelligent implant is a knee prosthesis, then the knee prosthesis still can function fully as a patient's knee; abilities lost, however, are the abilities to detect and to measure kinematic motion of the prosthesis, to generate and to store data representative of the measured kinematic motion, and to provide the stored data to a base station or other destination external to the kinematic prosthesis.

The controller 1032 is configured to cause the IMU 1022 to measure, in response to a movement of the kinematic prosthesis with which the IRP 1003 is associated, the movement over a window of time (e.g., ten seconds, twenty seconds, one minute), to determine if the measured movement is a qualified movement, to store the data representative of a measured qualified movement, and to cause the RF transceiver 1026 to transmit the stored data to a base station or other source external to the prosthesis.

For example, the IMU 1022 can be configured to begin sampling the sense signals output from its one or more accelerometers and one or more gyroscopes in response to a detected movement within a respective time period (day), and the controller 1032 can analyze the samples to determine if the detected movement is a qualified movement. Further in example, the IMU 1022 can detect movement in any conventional manner, such as by movement of one or more of its one or more accelerometers. In response to the IMU 1022 notifying the controller 1032 of the detected movement, the controller can correlate the samples from the IMU to stored accelerometer and gyroscope samples generated with a computer simulation or while the patient, or another patient, is walking normally, and can measure the time over which the movement persists (the time equals the number of samples times the inverse of the sampling rate). If the samples of the accelerometer and gyroscope output signals correlate with the respective stored samples, and the time over which the movement persists is greater than a threshold time, then the controller 1032 effectively labels the movement as a qualified movement.

In response to determining that the movement is a qualified movement, the controller 1032 stores the samples, along with other data, in the memory circuit 1024, and may disable the IMU 1022 until the next time period (e.g., the next day or the next week) by opening the switch 1016 to extend the life of the battery 1012. The clock and power management circuit 1020 can be configured to generate periodic timing signals, such as interrupts, to commence each time period. For example, the controller 1032 can close the switch 1016 in response to such a timing signal from the clock and power management circuit 1020. Furthermore, the other data can include, e.g., the respective sample rate for each set of accelerometer and gyroscope samples, respective time stamps indicating the time at which the IMU 1022 acquired the corresponding sets of samples, the respective sample times for each set of samples, an identifier (e.g., serial number) of the implantable prosthesis, and a patient identifier (e.g., a number). The volume of the other data can be significantly reduced if the sample rate, time stamp, and sample time are the same for each set of samples (i.e., samples of signals from all accelerometers and gyroscopes taken at the same times at the same rate) because the header includes only one sample rate, one time stamp, and one set of sample times for all sets of samples. Furthermore, the controller 1032 can encrypt some or all of the data in a conventional manner before storing the data in the memory circuit 1024. For example, the controller 1032 can encrypt some or all of the data dynamically such that at any given time, same data has a different encrypted form than if encrypted at another time.

The stored data samples of the signals that the one or more accelerometers and one or more gyroscopes of the IMU 1022 generate can provide clues to the condition of the implantable prosthesis and the recovery state of the patient. For example, the data samples may be processed and analyzed at a remote server to determine one or more gait parameters that may be monitored overtime to assess patient recovery state and health. The gait parameters may include: cadence, stride length, walking speed, tibia range of motion, knee range of motion, step count and distance traveled. The data can also be analyzed to determine whether a surgeon implanted the prosthesis correctly, to determine the level(s) of instability and degradation that the implanted prosthesis exhibits at present, to determine the instability and degradation profiles over time, and to compare the instability and degradation profiles to benchmark instability and degradation profiles developed with stochastic simulation or data from a statistically significant group of patients.

Furthermore, the sampling rate, output data rate (ODR), and sampling frequency of the IMU 1022 can be configured to any suitable values. For example, the sampling rate may be fixed to any suitable value (e.g., to 100 Hz, 800 Hz, 1600, or 3200 Hz for accelerometers, and up to 100 Hz for gyroscopes), the ODR, which can be no greater than the sampling rate and is generated by “dropping” samples periodically, can be any suitable value such as 800 Hz, and the sampling frequency (the inverse of the interval between sampling periods) for qualified events can be any suitable value, such as twice per day, once per day, once per every 2 days, once per week, once per month, or more or less frequently. The sampling rate or ODR can be varied depending on the type of event being sampled. For example, to detect that the patient is walking without analyzing the patient's gait or the implant for instability or wear, the sampling rate or ODR can be 200 Hz, 25 Hz, or less. Therefore, such a low-resolution mode can be used to detect a precursor (a patient taking steps with a knee prosthesis) to a qualified event (a patient taking at least ten consecutive steps) because a “search” for a qualified event may include multiple false detections before the qualified even is detected. By using a lower sampling rate or ODR, the IMU 1022 saves power while conducting the search, and increases the sampling rate or the ODR (e.g., to 800 Hz, 1600, or 3200 Hz for accelerometers, and up to 100 Hz for gyroscopes) only for sampling a detected qualified event so that the accelerometer signal and gyroscope signals have sufficient sampling resolution for analysis of the samples for the intended purpose, e.g., detection of instability and wear of the prosthesis, patient progress, etc.

Still referring to FIG. 5, in response to being polled by a base station or by another device external to the intelligent implant, the controller 1032 generates conventional messages having payloads and headers. The payloads include the stored samples of the signals that the IMU 1022 accelerometers and gyroscopes generated, and the headers include the sample partitions in the payload (i.e., in what bit locations the samples of the x-axis accelerometer are located, in what bit locations the samples of the x-axis gyroscope are located, etc.), the respective sample rate for each set of accelerometer and gyroscope samples, a time stamp indicating the time at which the IMU 1022 acquired the samples, an identifier (e.g., serial number) of the implantable prosthesis, and a patient identifier (e.g., a number).

The controller 1032 generates data packets that include the messages according to a conventional data-packetizing protocol. Each packet can also include a packet header that includes, for example, a sequence number of the packet so that the receiving device can order the packets properly even if the packets are transmitted or received out of order.

The controller 1032 encrypts some or all parts of each of the data packets, for example, according to a conventional encryption algorithm, and error encodes the encrypted data packets. For example, the controller 1032 encrypts at least the prosthesis and patient identifiers to render the data packets compliant with the Health Insurance Portability and Accountability Act (HIPAA).

The controller 1032 provides the encrypted and error-encoded data packets to the RF transceiver 1026, which, via the RF filter 1028 and antenna 1030, transmits the data packets to a destination external to the implantable prosthesis. The RF transceiver 1026 can transmit the data packets according to any suitable data-packet-transmission protocol.

Still referring to FIG. 5, alternate embodiments of the electronics assembly 1010 are contemplated. For example, the RF transceiver can perform encryption or error encoding instead of, or complementary to, the controller 1032. Furthermore, one or both of the switches 1016 and 1018 can be omitted from the electronics assembly 1010. Moreover, the electronics assembly 1010 can include components other than those described herein and can omit one or more of the components described herein.

### Operational Modes of Intelligent Implant

In some embodiments, an IRP 1003 of an intelligent implant is configured to be placed in five different modes of operation. These modes include a:

Deep sleep mode: this mode places the IRP 1003 is in an ultra-low power state during storage to preserve shelf life prior to implantation.

Standby mode: this mode places the IRP 1003 into a low power state, during which the implant is ready for wireless communications with an external device.

Low-resolution mode: while in this mode, the IRP 1003 collects kinematic data corresponding to low resolution linear acceleration data for step counting and detection of significant motion. In some embodiments, the low-resolution mode is characterized by activation of a first set of sensors, e.g., a single accelerometer or a pedometer, of an IMU 1022 that enable the detection of steps using a sampling rate in the range of 12 Hz to 100 Hz. When in low-resolution mode, the IRP 1003 counts steps and sends significant motion notifications to the controller 1032. When exiting the low-resolution mode, the IMU 1022 reports a step count to the controller 1032.

Medium-resolution mode: while in this mode, the IRP 1003 collects kinematic data corresponding to both acceleration data and rotational data. Medium-resolution kinematic data is used to determine kinematic information of the patient, including for example, a set of gait parameters including cadence, stride length, walking speed, tibia range of motion, knee range of motion, step count and distance traveled; and gait classifications including normal walking, walking with a limp, walking with limited range of motion, and other abnormal gait patterns. In some embodiments, the medium-resolution mode is characterized by activation of a second set of sensors, e.g., three accelerometers together with three gyroscopes, of an IMU that enable the detection of acceleration and rotational velocity using a sampling rate in the range of 12 Hz to 100 Hz. This mode may be initiated when an unspecified detection of a significant motion event occurs during a configured medium-resolution window of the day, or by a manual command sent wirelessly from an external device, e.g. a base station.

High-resolution mode: while in this mode, the IRP 1003 collects kinematic data corresponding to acceleration data. High-resolution kinematic data is used to identify complications associated with the intelligent implant, including micromotion, contracture, aseptic loosening, infection, incorrect placement of the device, unanticipated degradation of the device, and undesired movement of the device. In some embodiment, the high-resolution mode is characterized by activation of a third set of sensors, e.g., three accelerometers, of an IMU 1022 that enable the detection of acceleration using a sampling rate in the range of 200 Hz to 5000 Hz. This mode may be initiated when a specified detection of a significant motion event occurs during a configured medium-resolution window of the day, or by a manual command sent wirelessly from an external device.

These five modes are used passively to autonomously collect data at varying sampling frequencies during the life of the intelligent implant without patient involvement. The intelligent implant may start collecting data on post-operative day 2 and has the capability to store up to 30 days of data in memory. Thereafter, data is transmitted to the cloud daily. If the data cannot be transmitted due to connectivity issues with a base station and the implant has reached its memory limit, new data will overwrite the oldest data. Additionally, the base station can store up to 45 days of transmitted data if it is not able to connect to the cloud but is still able to communicate with the implant locally.

### Example Kinematic Data Sampling and Scheduling

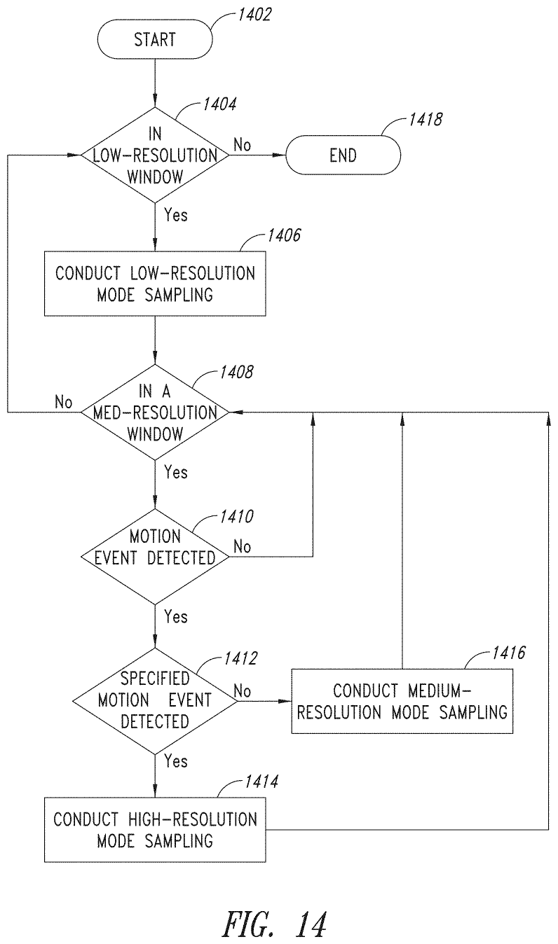

With reference to FIG. 14, a method of sampling data from an implantable reporting processor (IRP) of an intelligent implant in the form of a knee prosthesis is described. The method may be performed by the implantable reporting processor 1003 of FIG. 5 that is configured to sample data in each of a low-resolution mode, a medium-resolution mode, and a high-resolution mode. As previously described, a low-resolution mode may be characterized by activation of a first set of sensors of an IMU that enable the detection of steps using a sampling rate in the range of 12 Hz to 100 Hz; a medium-resolution mode may be characterized by activation of a second set of sensors of an IMU that enable the detection of acceleration and rotational velocity using a sampling rate in the range of 12 Hz to 100 Hz; and a high-resolution mode may be characterized by activation of a third set of sensors of an IMU that enable the detection of acceleration using a sampling rate in the range of 200 Hz to 5000 Hz.

Continuing with FIG. 14, at block 1402 a sampling session starts. The sampling session may be scheduled to occur based on a master sampling schedule programmed into the IRP 1003. In some embodiments, the master sampling schedule has a duration of a number of years from a calendar start date. For example, the number of years may be three. The master sampling schedule includes a calendar schedule that defines when data sampling will occur. In one embodiment, the periodic sampling is a daily sampling that is conducted in accordance with a daily sampling schedule. Accordingly, in this embodiment, the method of sampling data of FIG. 14 may occur on a daily basis. The IRP 1003 is configured to allow for disabling of the master sampling schedule.

At block 1404, the IRP 1003 determines if the present time is within a low-resolution window established by the daily sampling schedule. The low-resolution window may be defined by a start time and an end time. The low-resolution window may be a portion of a 24-hour period, and may have an associated duration limit. For example, the low-resolution window may be limited to a maximum duration of 18 hours.

At block 1406, if the IRP 1003 determines the present time is within a low-resolution window, the process proceeds to block 1406, where the IRP conducts low-resolution sampling. Alternatively, if the IRP 1003 determines the present time is not within a low-resolution window, the process proceeds to block 1418 where the sampling session ends.

Returning to block 1406, the IRP 1003 conducts low-resolution sampling during the low-resolution window by detecting and counting steps of the patient. The low-resolution sampling may be continuous throughout the low-resolution window. To this end, the IRP 1003 may enable an accelerometer of the IMU 1022 to provide signal samples from which steps of the patient may be detected. The low-resolution sampling rate may be in the range of 12 Hz to 100 Hz. In some embodiments, the IRP 1003 maintains a cumulative count of the steps that have been detected during each of a plurality of portions of the low-resolution window in its memory circuit 1024. For example, the IRP 1003 may maintain a cumulative count of step for each hour of the low-resolution window.

Continuing with FIG. 14, at block 1408, the IRP 1003 determines if the present time is within a medium-resolution window established by the daily sampling schedule. The IRP 1003 does this determining concurrently with low-resolution mode sampling. A medium-resolution window may be defined by a start time and an end time, where the start time of the medium-resolution window is within the low-resolution window. In some embodiments the daily sampling schedule defines a plurality of different medium resolution windows, each of which is defined by a start time that is within the low-resolution window, and an end time. There may a maximum number of allowable individual medium-resolution windows within a daily sampling schedule. For example, in one configuration there are a maximum of three individual medium-resolution windows. These individual medium-resolution windows may be scheduled to be spaced apart within the daily schedule or they may be scheduled such that there is some overlap between the windows. In some embodiments the duration of each individual medium-resolution window is in the range of 5-30 seconds. In some embodiments the duration of a medium-resolution window is 10 seconds. A medium-resolution sampling for the duration of time is referred to herein as a “medium-resolution bout.”

At block 1408, if the IRP 1003 determines the present time is within a medium-resolution window, the process proceeds to block 1410, where the IRP detects for a significant motion event. Alternatively, if the IRP 1003 determines the present time is not within a medium-resolution window, the process returns to block 1404 where the IRP determines if the present time is still within a low-resolution window.

Returning to block 1410, the IRP 1003 detects for a significant motion event by sampling the analog signals output from a second set of sensors of the IMU. In some embodiment, the second set of sensors include the accelerometers and the gyroscopes of the IMU. The IMU 1022 samples the analog signals at the same sampling rate associated with the medium-resolution mode. For example, the IMU 1022 samples the analog signals output from all of the x, y, and z accelerometers and gyroscopes in the range of 12 Hz to 100 Hz. Furthermore, the controller 1032 causes the IMU 1022 to sample the analog signals output from the accelerometers and gyroscope for a finite time, such as, for example, during a time window of ten seconds.

Continuing with block 1410, the controller 1032 determines whether the samples that the IMU 1022 obtained are samples of a significant motion event, such as the patient 1070 walking with the implanted knee prosthesis 1072. For example, the controller 1032 may correlate the respective samples from each of one or more of the accelerometers and gyroscopes with corresponding benchmark samples (e.g., stored in memory circuit 1024 of FIG. 5) of a significant motion event, compare the correlation result to a threshold, and determines that the samples are of a qualified event if the correlation result equals or exceeds the threshold or determines that the samples are not of a significant motion event if the correlation result is less than the threshold. Alternatively, the controller 1032 may perform a less-complex, and less energy-consuming determination by determining that the samples are of a significant motion event if, for example, the samples have a peak-to-peak amplitude and a duration that could indicate that the patient is walking for a threshold length of time. In another example, a significant motion event may correspond to a change of acceleration exceeding a threshold, and detecting the significant motion event comprises detecting a first change in acceleration that exceeds the threshold, and after a wait time, detecting a second change in acceleration that also exceeds the threshold.

In some embodiments, detection of a significant motion event is based on a set of programmable parameters including a significant motion threshold, a skip time, and a proof time. A significant motion is a change in acceleration as determined from the samples of one or more of the accelerometers. The controller 1032 detects for an initial change in velocity that exceeds the programmed significant motion threshold. Upon such detection, a conditional detection of a significant motion event is deemed to have occurred. The controller 1032 then waits for a number of seconds specified by the skip time parameter, and then detects for a subsequent change in velocity that exceeds the programmed significant motion threshold. Detection for the subsequent change in velocity occurs during a number of seconds specified by the proof time parameter. If a subsequent change in velocity is detected during the proof time, then a confirmed detection of a significant motion event is deemed to have occurred. Note that the subsequent change in velocity represents a change in velocity relative to the initial change in velocity. In other words, the subsequent change in velocity is a different value than the initial change in velocity.

In one configuration, the default setting for the significant motion threshold is in the range of 2 mg and 4 mg, the default setting for the skip time is in the range of 1.5 seconds to 3.5 seconds, and the default setting for the proof time is in the range of 0.7 seconds and 1.3 seconds. As described further below in the Configuration Management section of this disclosure, these programmable parameters may be adjusted based on analyses of the number of significant motion events confirmed by an IMU 1022.

Continuing with block 1410, if the IRP 1003 does not detect a significant motion event, the process returns to block 1408 where the IRP determines if the present time is still within the present medium-resolution window. Alternatively, if the IRP 1003 detects a significant motion event, the process proceeds to block 1412, where the IRP determines if this detection is a specified occurrence, or a specified detection of the significant motion event within the present medium-resolution window. A specified detection may be, for example, a first or initial detection of a significant motion event during the current medium-resolution window. In some embodiments, the specified detection may be a particular one, e.g., the second, third, etc., in a sequence of detections of significant motion events during the current medium-resolution window.

At block 1412, if the IRP 1003 determines that the detection is a specified detection of a significant motion event within the current medium-resolution window, the process proceeds to block 1414, where the IRP conducts high-resolution sampling for a duration of time. A high-resolution sampling for the duration of time is referred to herein as a “high-resolution bout.”

Alternatively, if the IRP 1003 determines that the detection is not a specified detection of a significant motion event within the current medium-resolution window, but instead is an unspecified detection, the process proceeds to block 1416, where the IRP conducts medium-resolution sampling. An unspecified detection may be a subsequent detection of a significant motion event that occurs after the specified detection. For example, if the specified type is defined as an initial detection of a significant motion event within a current medium-resolution window, then an unspecified detection would be any detection in the current medium-detection window that occurs after the initial detection.

Returning to block 1414, the IRP 1003 conducts high-resolution sampling by generating and storing signals indicative of three-dimensional movement. To this end, the IRP 1003 may enable a plurality of accelerometers of the IMU 1022 to provide respective signals, wherein the signals represent acceleration information of the intelligent implant and the patient. In some embodiments, three accelerometers of the IMU 1022 are activated for high-resolution sampling to provide acceleration information along three axes of the IMU. The high-resolution sampling rate may be in the range of 200 Hz to 5000 Hz. This acceleration information may be processed by the controller 1032 or transmitted to an external device for analysis based on that data, which may be used to identify and/or address problems associated with the implanted medical device, including incorrect placement of the device, unanticipated degradation of the device, and undesired movement of the device, such as described in PCT Publication No. WO 2020/247890, the disclosure of which is incorporated herein.

In one configuration, the daily sampling schedule limits high-resolution sampling to a predetermined number of times per day. In one configuration, the number of times per day is one. The daily sampling schedule may also set the duration of the high-resolution sampling. For example, the high-resolution sampling may occur for a duration in the range of 1 second to 10 seconds.

Returning to block 1416, the IRP 1003 conducts medium-resolution sampling by generating and storing signals indicative of three-dimensional movement. To this end, the IRP 103 may enable a plurality of accelerometers of the IRP and a plurality of gyroscopes of the IRP to provide respective signals. The signals from the accelerometers represent acceleration information of the intelligent implant and the patient, while the signals from the gyroscopes represent angular velocity information of the intelligent implant and the patient. In some embodiments, three accelerometers of the IMU 1022 are activated for medium-resolution sampling to provide acceleration information along three axes of the IMU 1022. In some embodiments, three gyroscopes of the IMU 1022 are activated for medium-resolution sampling to provide angular velocity information about three axes of the IMU. Collectively, the acceleration information and the angular velocity information represent kinematic information of the patient. This information may be processed by the controller 1032 or transmitted to an external device for processing, to determine kinematic information of the patient, including for example, a set of gait parameters including range of motion, step count, cadence, stride length, walking speed, and distance traveled.

The medium-resolution sampling rate may be in the range of 12 Hz to 100 Hz. The medium-resolution sampling may be conducted a limited number of times during the medium-resolution window. In one configuration, the daily sampling schedule limits medium-resolution sampling to once per medium-resolution window. The daily sampling schedule may also set the duration of the medium-resolution sampling. For example, the medium-resolution sampling may occur for a duration in the range of 5 seconds to 30 seconds. A medium-resolution sampling for the duration of time is referred to herein as a “medium-resolution bout.”

In addition to the schedule periodic data sampling of FIG. 14, the IRP 1003 may be configured to sample data in response to the receipt of an on-demand start command. The on-demand start command may be received by the IRP 1003 from an external device. The on-demand start command may specify the sampling mode, e.g., medium-resolution sampling (block 1416 of FIG. 14) or high-resolution sampling (block 1414 of FIG. 14), and a duration of the sampling, which may be in the range of 1 seconds to 30 seconds. The start command may also specify the sampling rate.

### Kinematic Data System

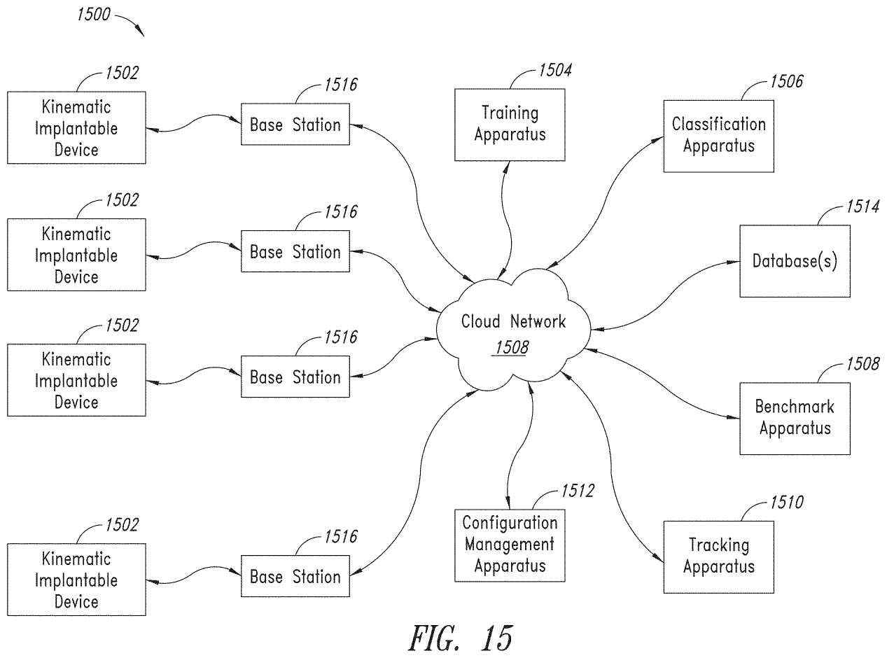

FIG. 15 is a block diagram of a system 1500 that obtains and processes kinematic data from intelligent implants and uses the data to train classification models (or outcome models), to classify motion activity associated with intelligent implants as different types of movements, to track patient recovery and/or implant conditions and/or other outcomes, and to configure implants to sense motion activity. This system 1500 may also or alternatively be used to obtain and process kinematic data from a wearable device of the present disclosure. The system 1500 includes a number of intelligent implants in the form of kinematic implantable devices 1502, a training processor 1504 (also referred to as a training apparatus), a classification processor 1506 (also referred to as a classification apparatus), a tracking standard processor 1508 (also referred to as a benchmark apparatus), a tracking processor 1510 (also referred to as a tracking apparatus), a configuration management processor 1512 (also referred to as a configuration management apparatus), and a database 1514.

As described in detail below, the system 1500 may use the kinematic data, together with other data such as demographic data, medical data, etc., to train classification models to classify motion activity. In addition to (or as an alternative to) training models to classify motion activity, the system 1500 may train classification models (or outcome models) to provide other outcomes. For example, an outcome model may be trained to provide other diagnostic or prognostic outcomes such as risk of infection, or implant loosening, or likelihood of full recovery, or estimated total cost of treatment.

Continuing with FIG. 15, the kinematic implantable devices 1502 are configured to collect data including operational data of the device along with kinematic data associated with particular movement of the patient or particular movement of a portion of the patient's body, for example, one of the patient's knees. The kinematic implantable devices 1502 are further configured to provide datasets of collected data to the database 1514. In some embodiments, datasets from kinematic implantable devices 1502 are communicated to one or more base stations 1516, which subsequently communicate the datasets to the database 1514 over a cloud network 1508. In some embodiments, datasets may be transmitted directly to any one of the training processor 1504, the classification processor 1506, the tracking standard processor 1508, the tracking processor 1510, the configuration management processor 1512.

As previously described, the kinematic implantable devices 1502 include one or more sensors to collect information and kinematic data associated with the use of the body part to which the kinematic implantable device 1502 is associated. For example, the kinematic implantable device 1502 may include an inertial measurement unit that includes gyroscope(s), accelerometer(s), pedometer(s), or other kinematic sensors to collect acceleration data for the medial/lateral, anterior/posterior, and anterior/inferior axes of the associated body part; angular velocity for the sagittal, frontal, and transvers planes of the associated body part; force, stress, tension, pressure, duress, migration, vibration, flexure, rigidity, or some other measurable data.

The kinematic implantable device 1502 collects data at various different times and at various different rates during a monitoring process of the patient. In some embodiments, the kinematic implantable device 1502 may operate in a plurality of different phases over the course of monitoring the patient so that more data is collected soon after the kinematic implantable device 1502 is implanted into the patient, but less data is collected as the patient heals and thereafter.

In one non-limiting example, the monitoring process of the kinematic implantable device 1502 may include three different phases. A first phase may last for four months where kinematic data is collected once a day for one minute, every day of the week. After the first phase, the kinematic implantable device 1502 transitions to a second phase that lasts for eight months and collects kinematic data once a day for one minute, two days a week. And after the second phase, the kinematic implantable device 1502 transitions to a third phase that last for nine years and collects kinematic data one day a week for one minute for the next nine years.

Along with the various different phases, the kinematic implantable device 1502 can operate in various modes to detect different types of movements. In this way, when a predetermined type of movement is detected, the kinematic implantable device 1502 can increase, decrease, or otherwise control the amount and type of kinematic data and other data that is collected.

In one example, the kinematic implantable device 1502 may use a pedometer to determine if the patient is walking. If the kinematic implantable device 1502 measures that a determined number of steps crosses a threshold value within a predetermined time, then the kinematic implantable device 1502 may determine that the patient is walking. In another example, the kinematic implantable device 1502 may use a step count gait parameter to determine if the patient is walking. In either case, in response to a determination that the patient is walking, the amount and type of data collected can be started, stopped, increased, decreased, or otherwise suitably controlled. The kinematic implantable device 1502 may further control the data collection based on certain conditions, such as when the patient stops walking, when a selected maximum amount of data is collected for that collection session or bout, when the kinematic implantable device 1502 times out, or based on other conditions. After data is collected in a particular session, the kinematic implantable device 1502 may stop collecting data until the next day, the next time the patient is walking, after previously collected data is offloaded (e.g., by transmitting the collected data to the base station 1516), or in accordance with one or more other conditions.

The amount and type of data collected by a kinematic implantable device 1502 may be different from patient to patient, and the amount and type of data collected may change for a single patient. For example, a medical practitioner studying data collected by the kinematic implantable device 1502 of a particular patient may adjust or otherwise control how the kinematic implantable device collects future data.

The amount and type of data collected by a kinematic implantable device 1502 may be different for different body parts, for different types of movement, for different patient demographics, or for other differences. Alternatively, or in addition, the amount and type of data collected may change overtime based on other factors, such as how the patient is healing or feeling, how long the monitoring process is projected to last, how much battery power remains and should be conserved, the type of movement being monitored, the body part being monitored, and the like. In some cases, the collected data is supplemented with personally descriptive information provided by the patient such as subjective pain data, quality of life metric data, co-morbidities, perceptions or expectations that the patient associates with the kinematic implantable device 1502, or the like.

In various embodiments, a base station 1516 pings its associated kinematic implantable device 1502 at periodic, predetermined, or other times to determine if the kinematic implantable device 1502 is within communication range of one or more of the home base station. Based on a response from the kinematic implantable device 1502, one or more of the base station 1510 determines that the kinematic implantable device is within communication range, and the kinematic implantable device can be requested, commanded, or otherwise directed to transmit the data it has collected to the base station 1510.

Along with transmitting datasets to the database 1514 over the cloud network 1508, the base station 1516 may also obtain data, commands, or other information from the configuration management processor 1512 via the cloud network. The base station 1516 may provide some or all of the received data, commands, or other information to the kinematic implantable device 1502. Examples of such information include, but are not limited to, updated configuration information, diagnostic requests to determine if the kinematic implantable device 1502 is functioning properly, data collection requests, and other information.

The database 1516 may aggregate data collected from the kinematic implantable devices 1502, and in some cases personally descriptive information collected from a patient, with data collected from other kinematic implantable devices, and in some cases personally descriptive information collected from other patients. In this way, the system 1500 creates and maintains a variety of different metrics regarding collected data from each of a plurality of kinematic implantable devices that are implanted into separate patients.

In embodiments disclosed herein, this information may be used by the training processor 1504 to train machine-learned classification models. The information may be used by the classification processor 1506 to classify motion activity associated with intelligent implants as different types of movements. The information may be used by the tracking standard processor 1508 to generate a standard dataset that provides information for tracking the recovery of a subject patient relative to a similar patient population or the tracking the condition of a surgical implant. The information may be used by the tracking processor 1510 to track patient recovery and/or implant conditions. The information may be used by the configuration management processor 1512 to optimize and adjust the configuration of implants to sense motion activity.

Having described the general function of the processors of the system of FIG. 15, more detailed description of these processors follows:

### Training Apparatus

Disclosed herein is a training apparatus that processes a (potentially large) collection of patient datasets across a patient population to train a machine-learning model to classify subsequent instances of sensor data (referred to herein as kinematic data) as one of a particular type of movement. As described further below, the patient datasets may include various types of data, including kinematic data that is obtained from one or more sensors of an IMU 1022. To improve the accuracy of the machine-learning model in classifying movement type, data preprocessing measures are taken to ensure quality and consistency of the kinematic data across the patient population that is used to train the machine-learning model. To this end:

1) Sensor calibration: each sensor of an IMU 1022 is calibrated with up to 24 coefficients to account for the variability in the manufacturing process of the sensor and IMU.

2) Unit standardization: raw kinematic data is standardized to common physical units (seconds for time interval, meters per second square for accelerometer, degrees per second for gyroscope) so that data with different sampling frequencies and scale settings can be analyzed together sensibly.

3) Alignment standardization: The orientation of the sensor relative to the body part, e.g., tibia, can vary from surgery to surgery. Accordingly, principal component analysis or other methods may be used to adjust for the variability in alignment.

With reference to FIG. 16A, in some embodiments a training apparatus 1504 for training a machine-learned classification model includes a data processing module 1602, a feature engineering module 1604, a machine-learning model 1606, and one or more optional labeling modules 1608.

### Data Processing and Feature Engineering

For purposes of a machine-learned classification model, the training apparatus 1504 obtains a number of patient datasets 1610 from across a patient population. Each patient dataset 1610, which may be obtained from the database 1514 of the system of FIG. 15, includes one or more records of motion activity of a body part of a particular patient in the patient population. In some embodiments, each individual record of motion activity in a patient dataset 1610 generally corresponds to one bout and includes several cycles of a motion activity sensed by a kinematic implantable device 1502. For example, the kinematic intelligent implant 1502 may be a knee replacement system for a partial or total knee arthroscopy (TKA) that includes a tibial extension and an IRP, the body part may be a tibia into which the IRP extends, and the associated motion activity may be walking, with each cycle corresponding to an individual step.

A patient dataset 1610 may include additional data that represent information upon which a machine-learned classification model may be trained. With reference to FIG. 16A and shown as inputs to the machine-learning module 1606, such data/information may include one or more of:

1) patient demographic data 1620, such as age, sex, weight, height, race, education, credit score, driving record, survey data, and geographic location;

2) patient medical data 1622, such as height, weight, body mass index (BMI), surgical procedure, medical device implanted, date of surgery, length of surgery, previous infection (MRSA), relevant baseline movement parameters, e.g., knee, hip, or shoulder parameters, in-clinic physical therapy frequency, bone density, pre-operation range of motion, manipulation, comorbidities, e.g., diabetes, osteoporosis, current smoking, lymphedema, malnutrition or inflammatory disease, and other patient conditions, e.g., brain aneurysms, physician/hospital comparison scores (e.g., from U.S. News & World Report, CMS Hospital Compare), Medicare/Medicaid payment information, economics (e.g. total cost of care);

3) device operation data 1624, such as device configuration, and sensor sampling rate for a record, e.g., low-resolution sampling at 1-25 HZ, medium-resolution sampling at 50 Hz, high-resolution sampling at 800 Hz);

4) clinical outcome data 1626, such as implant loosening, implant instability, stiffness, infection, revision surgery, pain, abnormal motions (e.g., limping), healing date, and patient reported outcome scores;

5) clinical movement data 1628, such as patient reported outcome measurements, and numeric pain rating scales;

6) non-kinematic data 1629, such as physiological measurements, anatomical measurements, and metabolic measurements, provided for example by glucose monitors, blood pressure monitors, chemistry sensors, metabolic sensors, and temperature sensors;

7) cluster labels 1630 assigned to kinematic features;

8) supervised labels 1632 assigned to kinematic features; and

9) kinematic features 1616, such as time-series variables, time-series waveforms, spectral distribution graphs, and spectral variables.

Regarding clinical movement type data 1628, this data characterizes a particular record of motion activity as a particular movement type. For example, the body part may be a tibia and the associated movement type for a record may be a normal movement (e.g., walking with a normal gait, running with a normal gait, walking up stairs with a normal gait, walking down stairs with a normal gait, walking up a slope with a normal gait, walking down a slope with a normal gait, biking) or an abnormal movement type (e.g., walking with a limp, walking with a limited range of motion, walking with a shuffle, walking with an assisted device (e.g., a cane, a walker, etc.), running with a limp, running with a limited range of motion, walking with an abnormal gait such an antalgic gait or a bow-legged gait. The clinical movement type data 1628 associated with a patient dataset 1610 may be obtained through clinical observation or through a patient diary or log of daily movement types.

Regarding non-kinematic data 1629, this data may correspond to any of the numerous data disclosed herein that may be obtained by any of the sensors disclosed herein. Examples of non-kinematic data 1629 include glucose levels sensed by a glucose monitor exposed the patient's bloodstream and blood pressure sensed by a pressure monitor.

Regarding a cluster label 1630, this data characterizes a particular record of motion activity as being within a particular cluster of similar records among a set of records. In some embodiments, the particular cluster label 1630 associated with a record may be previously determined by a clustering algorithm 1634 and stored in the patient dataset 1610. To this end, each record in a number of patient datasets 1610 may be kinematic data in the form of a signal corresponding to movement of the relevant body part. These signals may be graphically represented as time-series waveforms or spectral density graphs, and the clustering algorithm 1634 may be applied to the plurality of graphical representations to automatically separate the representations into groups or clusters of similar graphs based on a measure of similarity among the graphical representations in a group. Example known clustering algorithms 1634 that may be employed to cluster graphical representations of movement of a body part include k-means clustering and hierarchical clustering.

In some embodiments, once clustering of the set of graphical representations is complete, the clustering algorithm 1634 may automatically assign a generic cluster label 1630, e.g., cluster A, cluster B, etc., to each of the clusters. In some cases, a group of graphical representations that do not fall within a cluster may result from the operation of the clustering algorithm 1634. These graphical representations are referred to as “outliers,” and the clustering algorithm 1634 may accordingly automatically assign an “outlier” cluster label 1630 to this group.

In other embodiments, cluster labels 1630 may be manually assigned by an expert. To this end, the graphical representations of the one or more of the records within a cluster, determined by the clustering algorithm 1634, may be displayed on the user interface and display 1633. An expert may view the graphical representations and manually assign a cluster label 1630 to the cluster (and thereby each of the graphical representations within the cluster) through the user interface and display 1633. The cluster labels 1630 may be assigned based on visual similarities in a characteristic or pattern of the graphical representations in a cluster.

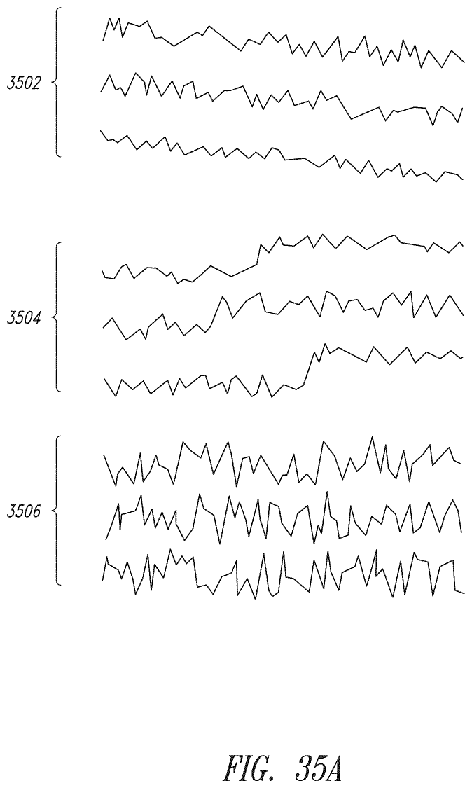

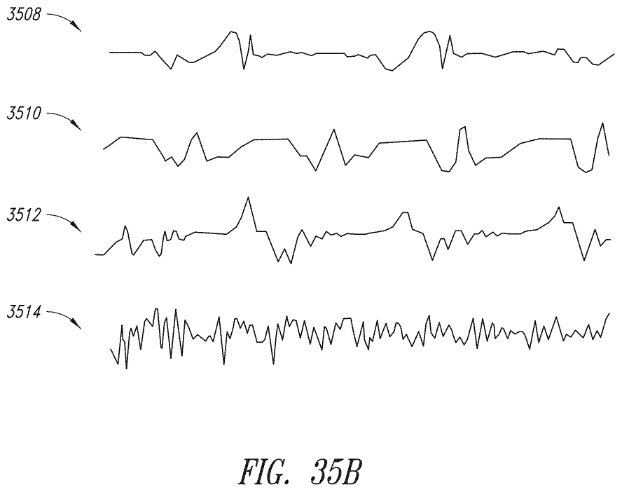

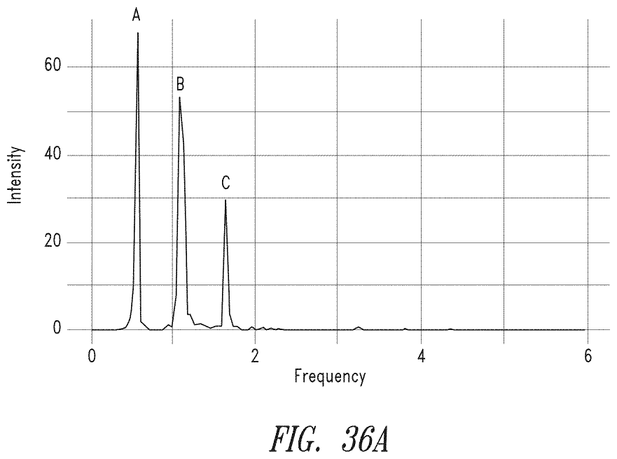

For example, with reference to FIG. 35A, the first cluster 3502 may be assigned a “decreasing” cluster label 1630 due to the downward slope of the time-series waveforms, the second cluster 3504 may be assigned a “jump” cluster label due to the jump in the time-series waveforms, and the third cluster 3506 may be assigned a “variable” cluster label 1630 due to the high rate of variation in the time-series waveforms. With reference to FIG. 35B, a cluster having time-series waveforms similar to the first waveform 3508 may be assigned a “stiffness” cluster label 1630, a cluster having time-series waveforms similar to the second waveform 3510 may be assigned a “short steps” cluster label 1630, a cluster having time-series waveforms similar to the third waveform 3512 may be assigned a “limping” cluster label 1630, and a cluster having time-series waveforms similar to the fourth waveform 3514 may be assigned a “micromotion” cluster label 1630. The foregoing a merely example of labels that may be assigned to a cluster. Numerous other labels descriptive of movement may be assigned to a cluster. Furthermore, a group of graphical representations that do not share similarities among themselves or with any cluster may be displayed. As noted above, these graphical representations are referred to as “outliers,” and the expert may accordingly assign an “outlier” cluster label 1630 to this group. Cluster labels other than movement type labels may be assigned. For example, labels such as: pain/no-pain, clinical outcome scores (e.g., WOMAC score), infection/non-infection, health care expenditures on a particular patient over a specified period of time.

Once labeling of the group of clusters is completed, the clustering algorithm 1634 associates the cluster label 1630 assigned to a particular group with each of the graphical representations in the particular group and with the corresponding record from which the graphical representations originated. The cluster label 1630 may be added to the relevant patient datasets 1610 and later provided as an input to the machine-learning model 1606.

Regarding the supervised label 1632, this data characterizes a particular record of motion activity as being a particular type of motion activity. In some embodiments, the particular supervised label 1632 associated with a record may be previously determined by an expert through a supervised labeling module 1636 and stored in the patient dataset 1610. To this end, each record in a number of patient datasets 1610 may be kinematic data in the form of a signal corresponding to movement of the relevant body part. These signals may be graphically represented as time-series waveforms or spectral density graphs and presented for visual observation on a user interface and display 1633.

In some embodiments, the graphical representations may identify one or more fiducial points or waveform features, e.g., local maxima and local minima, and zero crossings, with markers. An expert may view the graphical representations together with the fiducial point markers, if present, and manually assign a label to each of the graphical representations through the user interface and display 1633. For example, in the case of walking movement, the graphical representations of such movement may be labeled as (1) not walking, (2) walking with correctly placed fiducial markers, or (3) walking with incorrectly placed fiducial markers.

Once expert labeling of the number of graphical representations is complete, the supervised labeling module 1636 associates the assigned labels with its corresponding graphical representations and with the corresponding record from which the graphical representations originated. The supervised label 1632 may be added to the relevant patient dataset 1610 and later provided as an input to the machine-learning model 1606.

Regarding kinematic features 1616, with reference to FIGS. 16A, 16B, and 16C, for each obtained record of motion activity, the training apparatus 1504 processes the record and generates additional information, e.g., kinematic features, upon which a machine-learned model may be trained. The data processing module 1602 receives a record comprising raw kinematic data 1612 corresponding to movement of the body part and processes the data in one or more ways to provide data to the feature engineering module 1604, which in turn, processes the data further to extract or derive kinematic features 1616.

The raw kinematic data 1612 used to derive the kinematic features 1616 may be obtained from one or more sensors associated with the body part. The one or more sensors may be an external sensor or an implanted sensor or a combination of external sensors and implanted sensors. For example, the one or more sensors may be included in an IMU that is implanted within the body part, e.g., tibia. The sensor may be a gyroscope oriented relative to the body part and configured to provide raw kinematic data 1612 corresponding to angular velocity about a first axis relative to the body part. The sensor may be an accelerometer oriented relative to the body part and configured to provide raw kinematic data 1612 corresponding to acceleration along a first axis relative to the body part.

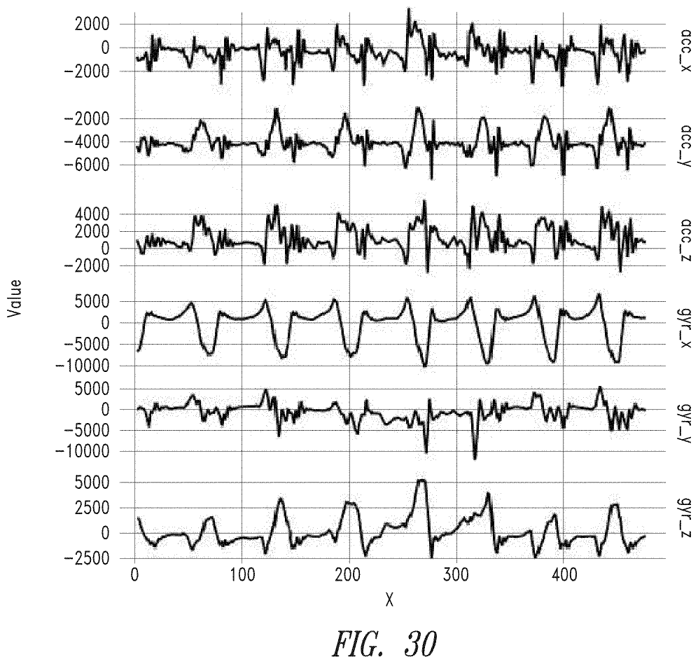

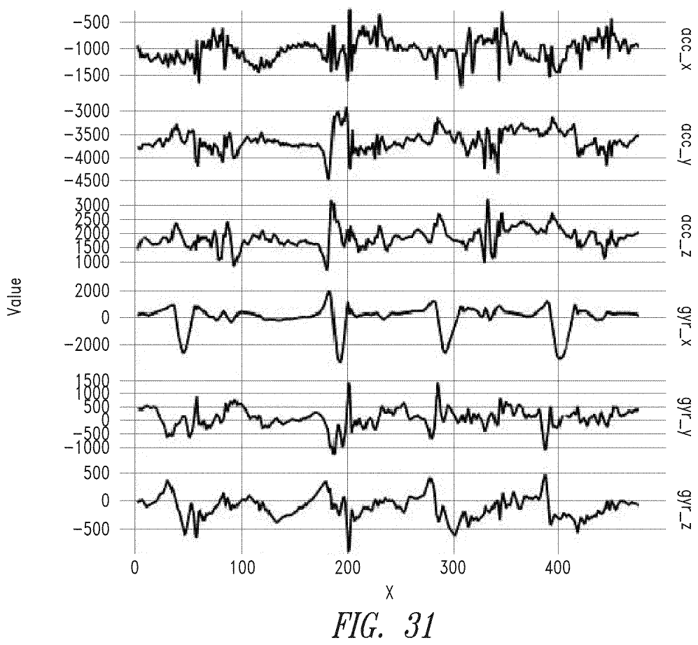

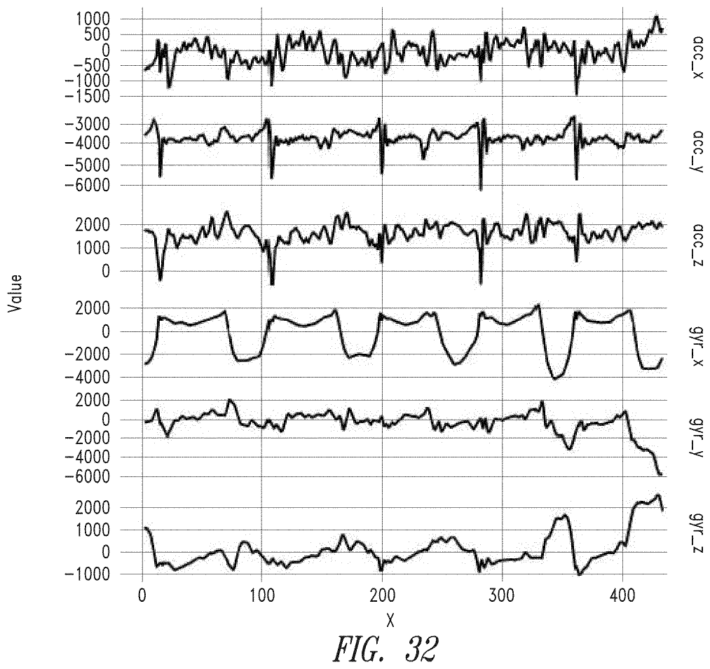

In one example embodiment, a gyroscope of an IMU provides raw kinematic data 1612 in the form of a gyroscope signal relative to the x-axis that is used to train a model to distinguish between a normal gait and an abnormal agit, e.g., walking with a limp, walking with a limited range of motion, etc. In some embodiments, each of three accelerometers and three gyroscopes of a six-channel IMU provide respective raw kinematic data 1612 in the form of gyroscope signals and accelerometer signals relative to a three-dimensional coordinate system that is used to train a model to distinguish between a normal gait and an abnormal agit, e.g., walking with a limp, walking with a limited range of motion, etc. FIG. 30 are illustrations of raw kinematic signals sensed across all channels of a six-channel IMU, during normal walking by a patient. FIG. 31 are illustrations of raw kinematic signals sensed across all channels of a six-channel IMU, while a patient is walking with knee pain. FIG. 32 are illustrations of raw kinematic signals sensed across all channels of a six-channel IMU, while a patient is walking with contracture (limited range of motion). In some embodiments, the IMU further includes three magnetometers that provide respective raw kinematic data 1612 in the form of magnetometer signals relative to a three-dimensional coordinate system. The magnetometer signals provide measures of the direction, strength, and/or relative change of a magnetic field. In this case, the IMU may be characterized as a nine-channel IMU.

In some embodiments, the raw kinematic data 1612 obtained from each sensor may be processed individually to generate kinematic features 1616 for training the machine-learning model 1606. In some embodiments, the raw kinematic data 1612 obtained from a set of sensors may be combined or fused to generate kinematic features 1616 for training the machine-learning model 1606. For example, the respective gyroscope signal for each of the x-axis, y-axis, z-axis that are captured during a same sampling window may be transformed into Euler angles using known sensor fusion algorithms, such as Kalman filtering. Likewise, the respective accelerometer signal for each of the x-axis, y-axis, z-axis that are captured during a same sampling window may be transformed using known sensor fusion algorithms into Euler angles. In another example, in the case of a six-channel IMU the three gyroscope signals and three accelerometer signals captured during the same sampling window may be transformed into three-channel Euler angles using known sensor fusion algorithms. In this approach accelerometer and gyroscope x-axis, y-axis, z-axis data is transformed into x-axis, y-axis, and z-axis Euler angles. In another example, in the case of a nine-channel IMU the three gyroscope signals and three accelerometer signals and the three magnetometer signals captured during the same sampling window may be transformed into three-channel Euler angles using known sensor fusion algorithms. In this approach accelerometer and gyroscope and magnetometer x-axis, y-axis, z-axis data is transformed into x-axis, y-axis, and z-axis Euler angles.

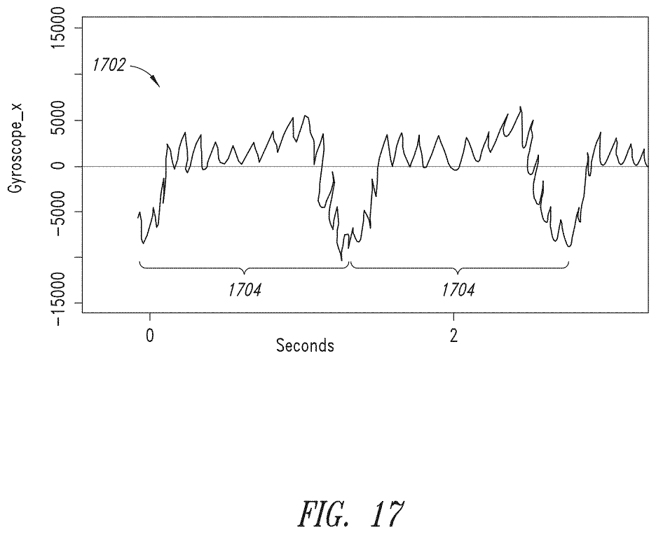

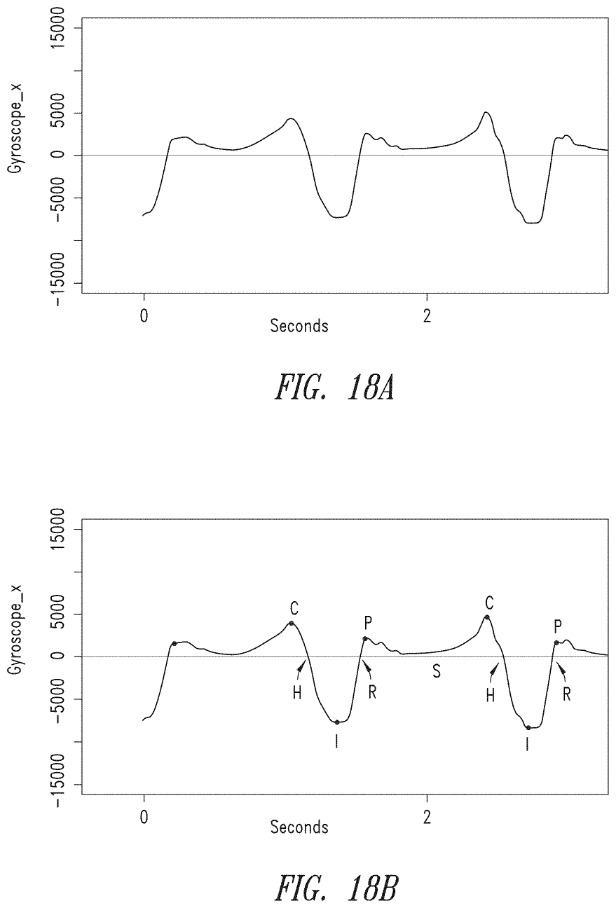

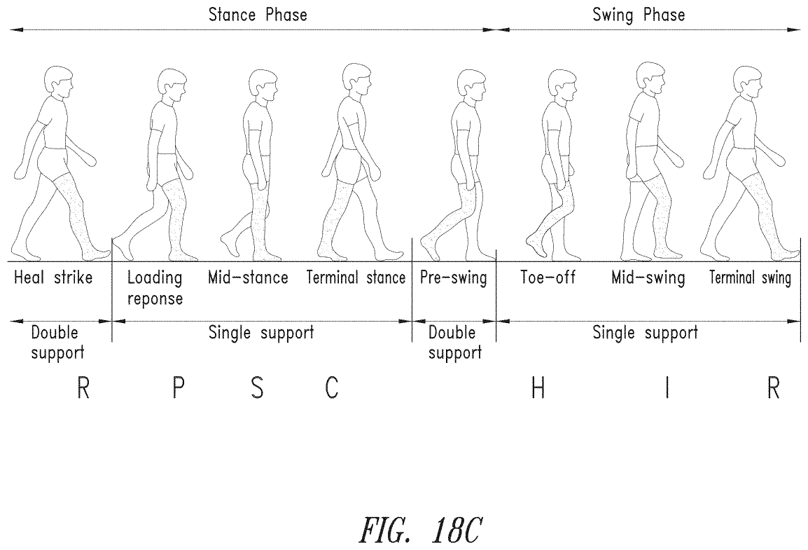

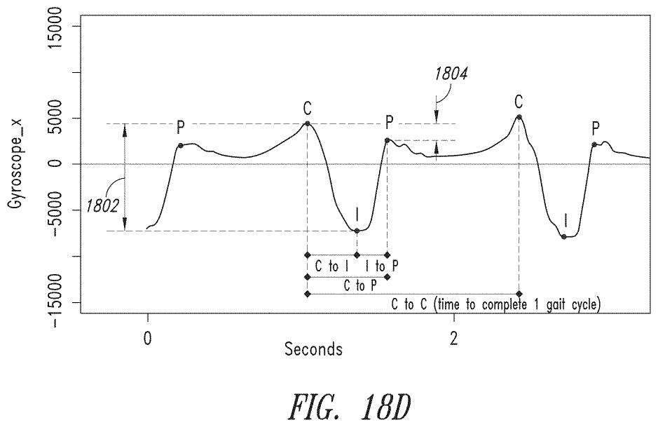

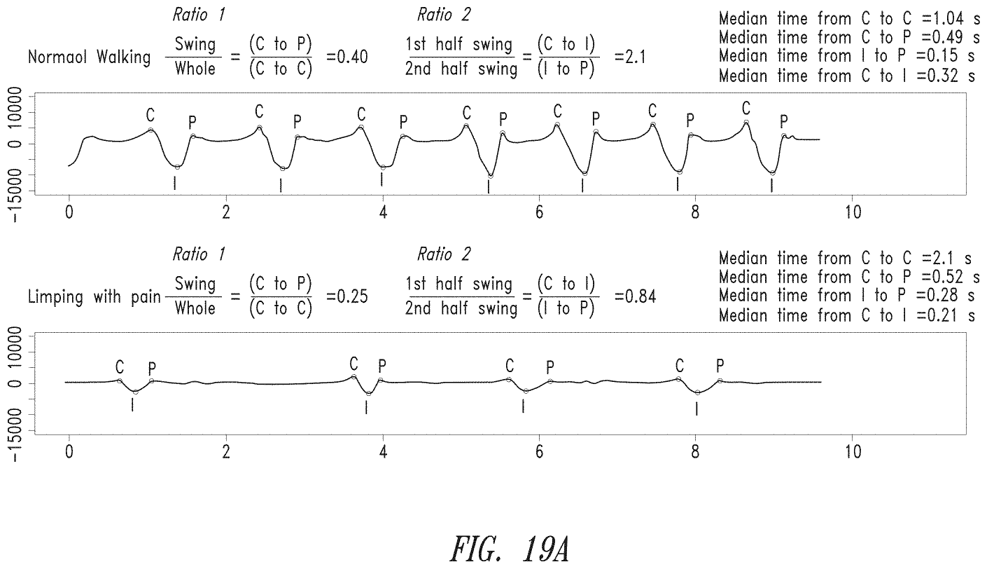

With reference to FIG. 16B, in some embodiments the data processing module 1602 includes a time-series waveform module 1640 and a frequency transformation module 1642. The time-series waveform module 1640 is configured to receive raw kinematic data 1612 and generate processed kinematic data 1614 in the form of time-series data 1650. For purpose of visual context, an example time-series waveform 1702 representation of raw kinematic data 1612 obtained from a knee replacement system is shown in FIG. 17, wherein the body part may be a tibia, the associated motion activity may be walking, and the time-series waveform includes a number of gait cycles. An example time-series waveform 1802 representation of processed kinematic data 1614, e.g., time-series data 1650, derived from the raw kinematic data 1612 that produced FIG. 17, is shown in FIG. 18A. The time-series waveform module 1640 may also be configured to generate processed kinematic data 1614 in the form of fused time-series data 1651. The frequency transformation module 1642 is configured to receive one or more of the timer-series data 1650 and the fused time-series data 1651 and transform the data into respective frequency data 1670.

With continued reference to FIG. 16B, the time-series waveform module 1640 includes a segmentation module 1646 and a smoothing module 1648. The segmentation module 1646 is configured to partition the motion activity, for example the gait activity as represented by the raw time-series waveform 1702 of FIG. 17, into individual segments 1704, each corresponding to a step. To this end, the segmentation module 1646 may use Fourier transformation, band-pass filtering, and heuristic rules to partition the time-series waveform into individual segments. The smoothing module 1648 is configured to receive each segment of the raw time-series waveform 1702 and to reduce the amount of noise in the segment. To this end, the smoothing module 1648 may use a smoothing technique, e.g., locally weighted smoothing (LOESS) or spline smoothing, to remove the noise from each of the segments. The final output of the time-series waveform module 1640 is time-series data 1650 that, as previously mentioned, may be represented as a smooth time-series waveform as shown in FIG. 18A.

The fusion module 1644 of the time-series waveform module 1640 is configured to receive the time-series data 1650 from the smoothing module 1648 and combine the data into fused time-series data 1651. The time-series data 1650 provided to the fusion module 1644 includes time-series data from two or more individual sensors. The fusion module 1644 combines the individual time-series data 1650 in a way that enables a determination of the position, trajectory, and the speed of the IMU, and this the body part with which the IMU is associated. To this end, the fusion module 1644 may “fuse” or combine the measured accelerations and angular velocities included in the time-series data 1650 to compute the orientations and positions of the IMU as a function of time. The orientations may be characterized by Euler angles. In some embodiments, the fuse module 1644 employs complementary, Kalman, Mahony, and Madgwick filters to are used to combine the measured accelerations and angular velocities.

As noted above, the respective gyroscope signal for each of the x-axis, y-axis, z-axis that are captured during a same sampling window may be processed by the fusion module 1644 to generate fused time-series data 1651 that represents Euler angle measurements as a function of time. Likewise, the respective accelerometer signal for each of the x-axis, y-axis, z-axis that are captured during a same sampling window may be processed by the fusion module 1644 to generate fused time-series data 1651 that represents Euler angle measurements as a function of time. In another example, in the case of a six-channel IMU the three gyroscope signals and three accelerometer signals captured during a same sampling window may processed by the fusion module 1644 to generate fused time-series data 1651 that represents Euler angle measurements as a function of time. In this case, the Euler angles represent the orientation of the IMU, which in turn represents the orientation of the body part with which the IMU is associated. By combining the time-series data 1650 from all sensors of a six-channel IMU it is possible to calculate the time evolution of the Euler angles relative to the direction of gravity. In another example, in the case of a nine-channel IMU the three gyroscope signals and three accelerometer signals and three magnetometer signals captured during a same sampling window may processed by the fusion module 1644 to generate fused time-series data 1651 that represents Euler angle measurements as a function of time. In this case, the Euler angles represent the orientation and direction of gravitational pull of the IMU, which in turn represents the orientation and direction of the body part with which the IMU is associated. By combining the time-series data 1650 from all sensors of a six-channel IMU it is possible to calculate the time evolution of the Euler angles relative to the direction of gravity.

With reference to FIG. 16C, the feature engineering module 1604 receives one or more of the processed time-series data 1650 and the processed fused time-series data 1651, and includes time-series waveform module 1642 and a time-series variable module 1660. The time-series waveform module 1642 is configured to generate a time-series waveform 1668 based on the time-series data 1650 and/or the fused time-series data 1651. An example of time-series waveform 1802 representation of time-series data 1650 is shown in FIG. 18A.

The time-series variable module 1660 receives the time-series waveform 1668 and is configured to further processes the time-series waveform to derive one or more time-series variables 1666. To this end, the variable-derivation module 1660 includes a fiducial point module 1662 configured to detect kinematic elements in the time-series waveform 1668. These elements may include one or more of the inflection points, zero crossing, local maxima and local minima.

With reference to FIG. 18B, in one configuration the fiducial point module 1662 identifies a set of six kinematic elements, each corresponding to a fiducial points C, H, I, R, P, or S in a time-series waveform representation of the time-series data 1650. These points are identified either by finding the x-coordinate (time) at which the signal crosses zero on the y-axis, or by identifying local minima or maxima values over different regions of the curve (e.g., point I could be defined as the most negative value between points H and R). In some embodiments, the time-series waveform may correspond to time-series data 1650 sensed by any one of the multiple sensing channels of an IMU as described above. For example, the time-series waveform may be based on time-series data 1650 sensed by a gyroscope with respect to the x-axis of the IMU. In some embodiments, the fiducial point module 1662 may apply a feature extraction algorithm to the time-series waveform to automatically detect the fiducial points. While the number of fiducial points described herein is six, more or less fiducial points may be detected. As general rules, the number and type of derivable time-series variables 1666 increases with the number of fiducial points, and a greater number and type of derivable time-series variables facilities detection and identification of a greater number of movement types, and differentiation between closely similar movement types.

Each fiducial point C, H, I, R, P, and S is described herein as generally corresponding to an event, point, or phase in a gait cycle. For example, and with reference to FIG. 18C, in the case of the body part being a tibia, movement of the body part may correspond to a gait cycle of a person as he is walking. The identified fiducial point C, H, I, R, P, and S in this case may generally correspond to a terminal stance “C”, a toe-off “H”, a mid-swing “I”, a terminal swing (just prior tor heel strike) “R”, a loading response “P”, or a mid-stance “S”.

With additional reference to FIG. 18B, at fiducial point C, as the toe lifts off, and the lower leg initiates swing phase, the tibia is at maximum angular velocity (as represented by the positive peak in the graph). Since this is the “commencement” of the stride, this fiducial point is called C. In terms of angular velocity as shown in FIG. 18B, fiducial point C corresponds to the point in a gait cycle where tibia positive angular velocity is maximum, which occurs during stance phase.

At fiducial point H, the tibia changes from positive rotation to negative rotation. Positive or clockwise rotation is defined as the proximal tibia moving anteriorly while relative to the distal tibia. Negative or counterclockwise rotation is defined as the proximal tibia moving posteriorly relative to the distal tibia. The angular velocity of zero is represented by the zero crossing in the graph. Since this occurs at the peak “height” of the tibia, this fiducial point is called H. In terms of angular velocity as shown in FIG. 18B, fiducial point H corresponds to the point in the gait cycle where the angular velocity is zero and the tibia changes from positive angular velocity to negative angular velocity.

At fiducial point I, the angular velocity of the tibia is the most negative it will become during swing phase of gait. Event I occurs at the negative local peak in the sagittal plane gyroscope graph. Since this corresponds to the “interval” between the two extremes of tibia motion, this fiducial point is called I. In terms of angular velocity as shown in FIG. 18B, fiducial point I corresponds to the point in the gait cycle where the angular velocity of the tibia is the most negative.

At fiducial point R, the angular velocity is zero and the tibia changes from a negative angular velocity to a positive angular velocity. Eventually, this forward reach stops and angular velocity is again zero (as represented by the zero crossing in the graph), and the tibia changes direction again. Since this occurs at the end of the forward “reach” of the tibia, this fiducial point is called R. In terms of angular velocity as shown in FIG. 18B, fiducial point R corresponds to the point in the gait cycle where the angular velocity is zero and the tibia changes from negative angular velocity to positive angular velocity.

At fiducial point P, angular velocity of the tibia increases quickly, but for a short period of time, as the tibia accelerates and places the heel on the ground. This brief increase in angular velocity of the tibia is represented by the peak P in the graph. Since this occurs upon initial contact or heel strike or foot strike or “placement” of the heel on the ground, this fiducial point is called P. In terms of angular velocity as shown in FIG. 18B, fiducial point P corresponds to the local maximum between points R and S.

At fiducial point S, the angular velocity of the tibia reaches a local minimum as the person begins to shift their weight forward, which unloads the leg, and so the tibia speeds up again. This local minimum of angular velocity if the tibia is represented by the flat region S of the graph. Since this occurs when the tibia “speeds” up, this fiducial point is called S. In terms of angular velocity as shown in FIG. 18B, fiducial point S corresponds to the local minimum between points P and C.

Returning to FIG. 16C, the variable calculation module 1664 receives information representative of the elements, e.g., fiducial points, detected by the fiducial point module 1662 and processes the information to generate time-series variables 1666. The information representative of the elements may be received in the form of a marked or tagged version of a time-series waveform, such as shown in FIG. 18B, that identifies the elements. Alternatively, the information representative of the elements may be received in the form of interval information independent of a waveform image. For example, the information representative of the elements may be received in the form of a matrix of the elements for all of the step cycles within each 10-second bout of data, wherein the matrix lists the element identifier, e.g., C, H, I, R, P, or S, the time of the event, and a corresponding measure, e.g., angular velocity, acceleration, etc., of the event.

The variable calculation module 1664 is configured to derive one or more time-series variables 1666 based on the fiducial points. To this end, the variable calculation module 1664 may calculate the one or more variables based on pairs of fiducial points. For example, with reference to FIG. 18D, variables corresponding to the time intervals between one or more of C and H, C and I, C and R, C and P, C and C, H and I, H and R, H and P, etc. may be calculated. Also, variables corresponding to peak-to-peak elevation or magnitude of C and H, C and I, C and R, C and P, C and C, H and I, H and R, H and P, etc. may be calculated. Variables corresponding to differences in elevation or magnitude of C and H, C and I, C and R, C and P, C and C, H and I, H and R, H and P, etc. may also be calculated.

Some of these variables describe aspects of the gait cycle that are easy to interpret. For example, with reference to FIG. 18D, the C-I variable 1802 in terms of peak-to-peak magnitude is the difference between the maximum forward angular velocity at toe-off (commencement C) and the maximum forward velocity when the tibia is at the bottom of its forward swing (interim velocity I) during a qualified step. The C-P variable 1804 in terms of magnitude is the difference between the maximum forward angular velocity at toe-off (commencement C) and the heel-strike (placement P).

The variable calculation module 1664 may also calculate time-series variables 1666 corresponding to ratios of one or more pairs of individual variables. For example, the ratios of the time intervals, e. g., H-to-R/C-to-P, C-to-P/C-to-C, C-to-I/I-to-P may be calculated. The ratios of magnitude differences, e.g., H-R/C-P, C-P/C-C, C I/I-P, may be calculated. The ratios of individual magnitudes, e.g., C/H, C/I, C/R, C/P, C/C, H/I, H/R, H/P, etc. may be calculated. The variable calculation module 1664 may also label each of the one or more calculated time-series variables 1666 with the movement type associated with the record that is being processed.

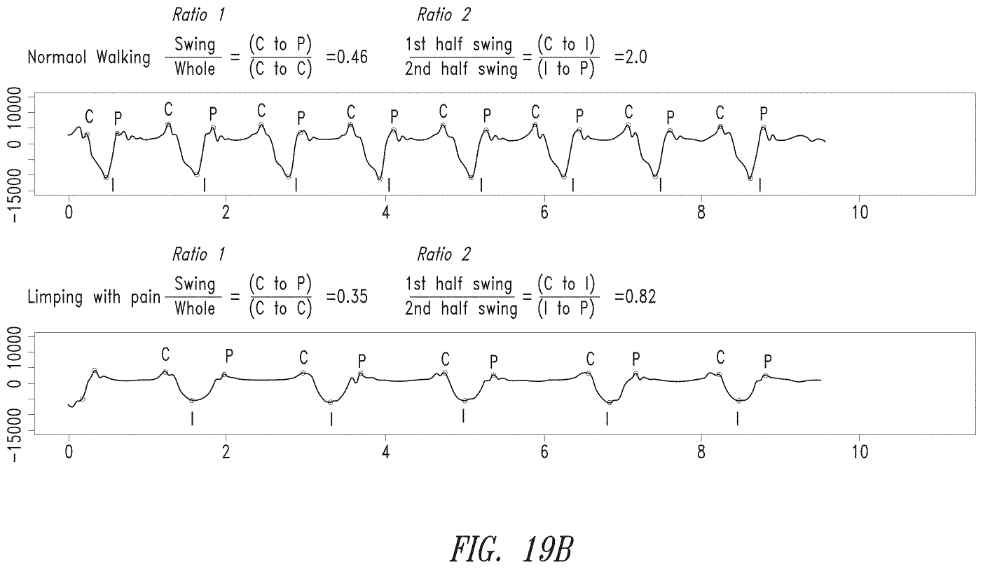

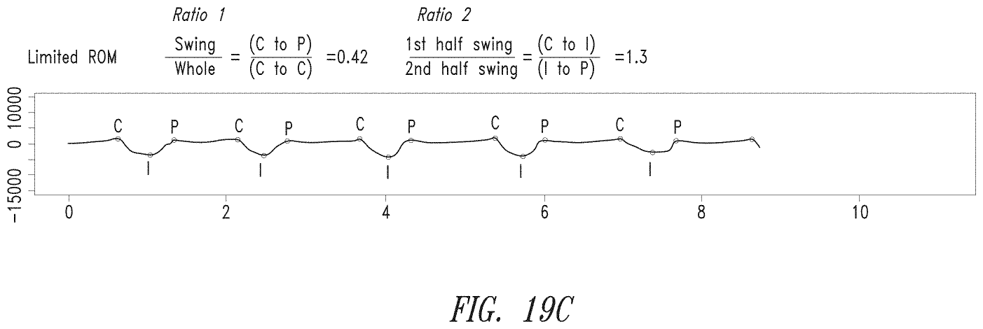

The time-series variables 1666 derived by variable calculation module 1664 may be used to distinguish between different types of movements. For example, with reference to FIG. 19A, which is an illustration of a kinematic signal sensed during normal walking by a patient relative to a kinematic signal sensed during limping with pain by the same patient, different time-series variables 1666 in the form of ratios are derived from different time-series variables corresponding to intervals. Comparing the respective ratios during normal walking and limping with pain indicates a difference significant enough to warrant the use of these measures as a means to assess a patient's condition and recovery. Furthermore, as it relates to machine-learning, the difference in respective ratios validates the use of time-series variables 1666 and associated labels, e.g., normal walking, walking with a limp, etc. for machine-learning.

With reference to FIG. 19B, which is an illustration of a kinematic signal sensed during normal walking by another patient relative to a kinematic signal sensed during limping with pain by the patient, different time-series variables 1666 in the form of ratios are derived from different time-series variables corresponding to intervals. Comparing the respective ratios during normal walking and limping with pain, again indicates a difference significant enough to warrant the use of these measures as a means to assess a patient's condition and recovery. Furthermore, as it relates to machine-learning, the difference in respective ratios validates the use of time-series variables 1666 and associated labels, e.g., normal walking, walking with a limp, etc. for machine-learning.

With reference to FIG. 19C, which is an illustration of a kinematic signal sensed during normal walking by a patient relative to a kinematic signal sensed during walking with a limited range of motion by the patient, different time-series variables 1666 in the form of ratios are derived from different time-series variables corresponding to intervals. Comparing the respective values during normal walking and walking with limited range of motion indicates a difference significant enough to warrant the use of these measures as a means to assess a patient's condition and recovery. Furthermore, as it relates to machine-learning, the difference in respective ratios validates the use of time-series variables 1666 and associated labels, e.g., normal walking, walking with a limp, etc. for machine-learning.

With reference to FIG. 16B, the frequency transformation module 1642 of the data processing module 1602 is configured to receive the segmented and smoothed time-series data 1650 and/or the fused time-series data 1651 from the time-series waveform module 1640. The frequency transformation module 1642 is configured to transform the time-series data 1650 and/or the fused time-series data 1651 into respective frequency data 1670 (individual sensor data or fused sensor data). To this end, the frequency transformation module 1642 may use Fourier transform and wavelet transform to transform time-domain data to frequency data or a mix of time and frequency data. Fourier transformation provides frequency information in highest possible resolution at the expense of not knowing the precise time each frequency occurs. Wavelet transformation provides not only the frequencies of the signal, but also the time at which each frequency occurs. Some resolution in frequency is given up but the timing information of those frequencies is retained. The wavelet transform may take the form of a 2D spectrum, with x and y-axis being the time and frequency, and the color indicating the intensity of the signal at a particular time and frequency.

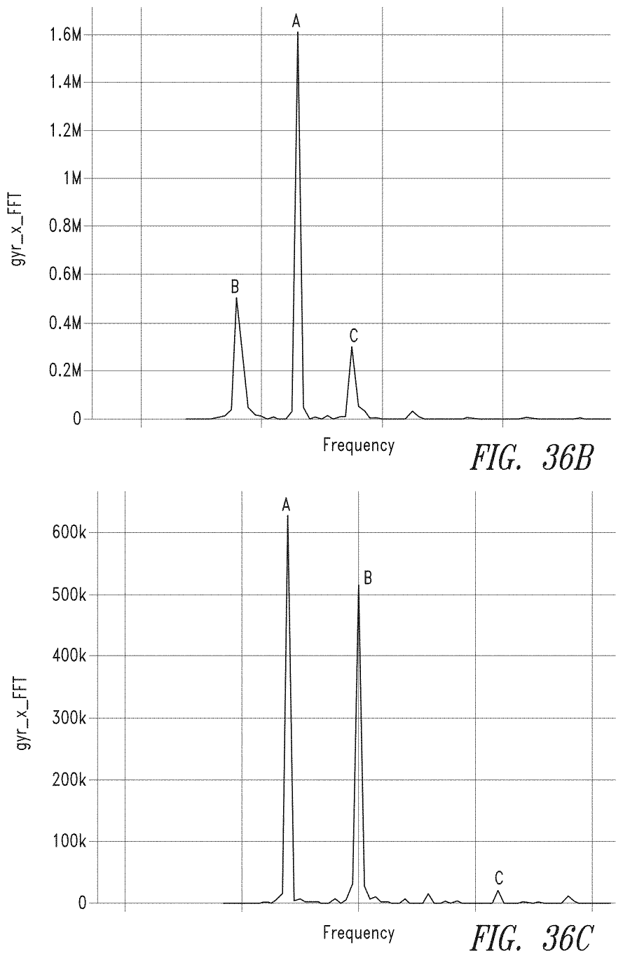

With reference to FIG. 16C, the feature engineering module 1604 receives the processed frequency data 1670 and includes a spectral distribution module 1672 and a spectral variable module 1674. The spectral distribution module 1672 is configured to generate a spectral distribution graph 1676 based on the frequency data 1670. Example spectral distribution graphs are shown in FIGS. 36A, 36B, and 36C.

The spectral variable module 1674 receives the spectral distribution graph 1676 and is configured to further processes the graph to derive one or more spectral variables 1678. To this end, the spectral variable module 1674 includes a spectral density module 1680 configured to identify one or more of peaks in a spectral distribution graph. For example, as shown in FIGS. 36A, 36B, and 36C, the top three spectral peaks may be identified as A, B, and C.

With reference to FIG. 36A, in one configuration the spectral density module 1680 detects a set of peaks A, B, and C in a spectral graph 3600 representation of the frequency data 1670. The spectral density module 1680 also characterizes each detected peak in terms of frequency and intensity. In some embodiments, the spectral density module 1680 may apply a feature extraction algorithm to the spectral graph to automatically detect the spectral peaks. While the number of peaks described herein is three, more or less peaks may be detected. As general rules, the number and type of derivable spectral variables 1678 increases with the number of peaks, and a greater number and type of spectral variables facilities detection and identification of a greater number of movement types, and differentiation between closely similar movement types.

The variable calculation module 1668 receives information representative of the peaks detected by the spectral density module 1680 and processes the information to generate spectral variables 1678. The information representative of the spectral peaks may be received in the form of a marked or tagged version of a spectral distribution graph, such as shown in FIG. 36A, that identifies the peaks. Alternatively, the information representative of the spectral peaks may be received in the form of spectral information independent of a graph image. For example, the information representative of the spectral peaks may be received in the form of a matrix of the peaks for each of the step cycles within a 10-second bout of data, wherein the matrix lists the frequencies present in the spectral density and their respective intensities.

The variable calculation module 1682 is configured to derive one or more spectral variables 1678 based on the spectral density information. To this end, the variable calculation module 1682 may calculate the frequency difference between pairs of peaks and/or the intensity differences between pairs of peaks. For example, with reference to FIG. 36A, the difference in frequency or intensity of peaks A and B, A and C, B and C may be calculated. The variable calculation module 1682 may also calculate spectral variables 1678 corresponding to ratios of the frequencies or intensities of the peaks A, B, and C. Calculated ratios may include for example, A/B, A/C, B/C. The variable calculation module 1664 may also label each of the one or more calculated spectral variables 1678 with the movement type associated with the record that is being processed.

The spectral variables 1678 derived by the variable calculation module 1682 may be used to distinguish between different types of movements. For example, with reference to FIGS. 36B and 36B, which are illustrations of a spectral distribution graph of a kinematic signal sensed during normal walking (FIG. 36B) by a patient relative to a spectral distribution graph of a kinematic signal sensed during limping (FIG. 36C) by the same patient, different spectral variables 1678 in the form of ratios are derived from different spectral variables corresponding to the intensity of the detected peak A, B, and C. Comparing the respective ratios during normal walking and limping indicates a difference significant enough to warrant the use of these measures as a means to assess a patient's condition and recovery. Furthermore, as it relates to machine-learning, the difference in respective ratios validates the use of spectral variables 1678 and associated labels, e.g., normal walking, walking with a limp, etc. for machine-learning.

The spectral variables 1678 derived by the variable calculation module 1682 may be used to distinguish between different types of implant conditions. For example, a high amount of high frequency content in a spectral distribution graph relative to other, lower frequency content may be indicative of implant micromotion or vibration that may be predictive of latter implant loosening.

### Model Training

With reference to FIG. 16D, the training apparatus 1504 trains the machine-learned model 1606 on the kinematic features 1616 to classify movement of a body part as a particular movement type. For example, as previously mentioned, the body part may be a tibia and the associated movement type may be a normal movement, e.g., walking or running, or an abnormal movement type, e.g., walking with a limp, walking with a limited range of motion, running with a limp, running with a limited range of motion. As noted above, the machine-learned model 1606 may be trained on other data. For example, to the extent such data is included in the patient dataset 1610 or otherwise available, the machine-learned model 1606 may be trained on the patient demographic data 1620; patient medical data 1622; device operation data 1624; clinical outcome data 1626; clinical movement data 1628; non-kinematic data 1629; cluster labels 1630; and supervised labels 1632.

The machine-learning model 1606 may employ one or more types of machine-learning techniques and machine-learning algorithms. For example, the machine-learned model 1606 may be based on one or more of statistical models, machine-learned models, and deep-learned models. In general terms, possible types of machine-learning techniques include supervised machine learning, unsupervised machine learning, reinforcement machine learning, and semi-supervised machine learning. Possible types of machine learning algorithms include generalized linear models, tree-based models, neural networks, clustering/similarities algorithms, and deep learning.

Unsupervised learning may be used if an outcome variable is not available, while supervised learning may be used if the outcome variable is available. A parametric model may be used if the data is sparse and/or the need for model interpretation is important. A non-parametric model may be used if the data is abundant, is non-linear, and/or prediction accuracy is more important than interpretation. A summary of the modeling techniques follows:

Unsupervised Learning: including 1) K-means clustering, and 2) hierarchical clustering

Supervised Learning—parametric models: including 1) generalized linear model, 2) generalized additive model, 3) generalized mixed effect model, and 4) survival model.

Supervised Learning—non-parametric models: including 1) tree-based models, such as random forest, and gradient boosted trees, and 2) neural network, such as convolutional neutral network, and recurrent neutral network.

### Example Model Training

In the following example a model may be trained in one of various ways to provide one or more diagnostic classifications (or outcomes) and/or prognostics classifications (or outcomes) within the context of a TKA. While the number of different types of classifications or outcomes within this setting is large, the examples described herein include: 1) infection, 2) pain (including a degree of pain), 3) movement type (limping or normal, including a degree of limping, e.g., mild, moderate, severe), 4) implant-loosening (including a degree of loosening, e.g., mild, moderate, severe), and 5) recovery state (fully recovered or not).

The model may be trained to provide a result as a binary classification (“this person has outcome X” vs “this person does not have outcome X”), or ordinal classification (e.g., “this person has mild/moderate/severe limping”). The model may be trained to provide a result as a risk score on a continuum (e.g., a number from 0 to 100, or a probability from 0.0 to 1.0). In some embodiments, a risk score is or represents a probability, log-odds, or odds of having a particular clinical outcome. For example, if the risk score is a probability, the model may define a risk score of over 0.15 as a high risk of having a particular clinical outcome, a risk score of between 0.10 and 0.015 as a moderate risk, and a risk score of under 0.10 as a low risk.

The model may be trained to achieve an accuracy level. For example, for binary classifications the model may be trained to have a sensitivity >90% and specificity>60%. For risk score classifications the model may be trained to have an area under the receiver operating characteristic (ROC) curve >0.75.

### Training Data Selection

Relevant data from the datasets of patients may be selected based on the above-identified outcomes of the model. This relevant data may, for example include: 1) kinematic features (time-series waveforms and there corresponding variables, spectral distributions graphs and there corresponding peaks, etc.); 2) demographic data, and 3) available clinical outcome data directed to the one or more outcomes of interest (e.g., infection, pain, movement, implant-loosening, and recovery state)

### Model Building and Validation

Using the selected data from the datasets, and using machine learning techniques, a model may be built to calculate a “risk score” for a new patient (one that the model has not seen before) using similar data of the new patient. In some embodiments, the risk score is defined as the probability, odds, or log-odds of a particular patient having the clinical outcome of interest. A model may be trained to predict a quantity other than a risk score/probability, depending on the outcome being modelled. For example, a model may be trained to predict a maximum “ROM” in degrees, based on the functional “tibia ROM” and other available data. Or in a blood sugar setting, a model may be trained to predict A1C levels, based on non-kinematic data, e.g., blood sugar sensor data.

Various modeling approaches may be used to build the classification model (or outcome model). As disclosed below, these approaches include statistical modeling, machine-learning methods, and deep learning methods.

### Statistical Models

A statistical model used to train the classification model may include, for example, a generalized linear model (GLM), a generalized additive model (GAM), a generalized additive model network (GAMnet), etc.

In statistical modeling, an outcome being modeled is structured as a mathematical formula composed of features and their weights. The modeling process produces estimates of the weights of the features in the mathematical formula. Note that some variables may have zero weights, which means they have no influence on the outcome. The process of identifying features with non-zero weights is known as feature selection. Because a statistical model has a mathematical formula, it is highly interpretable (which is a benefit to clinicians and patients).

A first example mathematical formula based on statistical modeling and a single feature follows:

y=α+β1*x1+error Eq. 34
where:
y=outcome or classification
α=by definition a part of a regression model. α is the value of y when x=0. Regression models estimate the value of all coefficients (alpha, beta1, beta2, . . . ) based on the patterns in the data, using optimization (or error minimization). This gives the “best fit line” which is a model for the data.
βn=weight of feature (xn)
xn=feature
error=an estimate of how well the line fits the data

An example mathematical formula based on Eq. 34 for an outcome corresponding to a risk of infection, and based on a single feature—“age”—follows:

Risk of infection=3.2+1.5*age+error Eq. 35

Another example mathematical formula based on Eq. 34 for an outcome corresponding to limping or not limping, and based on a single feature—“C-I interval”—follows:

Limping=4.1+2.2*C-I interval+error Eq. 36

With just 1 variable, the model is like an equation for a line. The alpha is the point at which the line intersects the y-axis (y-intercept), and the beta1 is the slope of the line. The error term is an estimate of how well the line fits the data.

The example numbers (3.2 and 1.5) in Eq. 35 and (4.1 and 2.2) in Eq. 36 are the numbers that make the line have the lowest amount of error and depend on the dataset. In some datasets, the relationship between age and risk of infection will be very strong, and thus it will be possible to calculate alphas and betas that fit the data very well and have very low errors. In other datasets, the relationship may be weak, and thus the alphas and betas will be different, and may not fit the data well, and will have very high errors.

A second example mathematical formula based on statistical modeling and a pair of features follows:

y=α+β1*x1+β2*x2+error Eq. 37
where:
y=outcome or classification
βn=weight of feature (xn)
xn=feature

Now, instead of a line (as in Eq. 34), the model has an equation for a surface in a 3-dimension graph.

An example mathematical formula based on Eq. 37 for an outcome corresponding to a risk of infection, and based on the pair of features—“age” and “C-I Interval”—follows:

Risk of infection=3.2+1.5*age+2.9*C-I Interval+error Eq. 38

An example mathematical formula based on Eq. 37 for an outcome corresponding to limping or not limping, and based on the pair of features—“age” and “C-I Interval”—follows:

Limping=4.1+2.2*age+3.3*C-I Interval+error Eq. 39

When structuring the mathematical formula, new features may be created by transforming or combining existing features to capture non-linear effects and/or interaction effects. Interaction effect is the phenomenon that the weight of feature A depends on the values of other features. The effect is known as 2-way interaction if feature A's weight depends on values of feature B. It is known as 3-way interaction if feature A's weight depends on values of features B and C. The complexity of the mathematical formula increases as non-linear and interaction features are added to the formula.

A third example mathematical formula based on statistical modeling based on a pair of features and an interaction or combination of features follows:

y=α+β1*x1+β2*x2+β3*x1*x2+error Eq.40
where:
y=outcome or classification
βn=weight of feature (xn), β3 is the coefficient that estimated for the interaction/combined term, x1*x2
xn=feature

The statistical modeling process estimates the weights (aka coefficients) of the features in the mathematical formula from the training data. The more complex the mathematical formula is, the more weights need to be estimated. The number of weights reasonably estimated is usually less than the number of observations containing the outcome of interest.

In the example mathematical formulas above, the features selected for training are age and C-I interval. The mechanism of feature selection varies greatly among different modeling techniques. For example, the technique of the “lasso” selects features by imposing a penalty on the weights of all features. At a low penalty, perhaps most features have non-zero weights. But at a high penalty, only the most influential features have non-zero weights. The “lasso” fits a series of models at a range of penalty levels. An independent validation dataset is used as a “judge” to decide at which penalty level the model performs the best (neither underfitting nor overfitting the data). The subset of features that “survive” under the optimum penalty level becomes the features in the model. This process of using an independent validation data set to pick the best model is known as “model selection.”

### Machine Learning Models

A machine learning model used to train the classification model may include, for example, a gradient boosting machine (GBM), a random forest, etc.

Unlike statistical models, there is no need to specify a mathematical formula to build a machine-learned model. Interaction effects among different features (e.g., age and C-I interval), non-linearity, and influential features are automatically discovered in the model training process.

A machine learning model is characterized by a set of tuning parameters. The optimal values for those parameters are found by training a series of models over a range of tuning parameter values. At each set of values, the model performance is assessed using an independent validation data set (the “judge”). The best model is the one that is characterized by the tuning parameters at their optimal values. Machine learning models can reveal which variables have been selected and their degree of influence on the outcome.

Deep Learning form of Machine Learning Models

A deep learning machine learning model used to train the classification model may include, for example, a neural network, etc.

Deep learning models are similar the machine learning models. Both provide high predictive accuracy for high-dimensional data or data with sophisticated interactions. Deep learning models may be trained on all data types including: 1) single values (e.g., demographic data, medical data, 2) engineered features (e.g., C-I intervals), and 3) higher-order data directly (e.g., kinematic time-series waveforms, spectral distribution graphs; without the need to engineer features). For example, a deep learning model may take raw kinematic data for a bout (6 or more channels and hundreds of values per channel) as input along with a patient's demographic/prognostic factors to identify patient characteristics. These “modes” (IMU and structured demographic/prognostic factors) are integrated into the model in a uniform way.

### Threshold for Binary Classification

If a model is being trained to provide a binary classification, then the model may use a probability threshold chosen for the diagnosis of outcome X. The threshold can be selected based on statistical, clinical, or operational considerations. One example a probability threshold may be chosen by: 1) calculating model performance (sensitivity and specificity) at every possible threshold, and 2) choosing a threshold which maximizes the desired sensitivity and specificity.

### Validation

To validate the trained model (and the threshold if applicable), the model is applied to a new set of patients. If the accuracy of the model meets the pre-specified accuracy requirements, then the model has passed validation.

If the model provides a binary classification, the model may be validated by:

1) Calculating the risk score for each new patient; in some embodiments this is the probability, odds, or log-odds that the patient has the outcome of interest.

2) Determining the classification for each patient based on the patient's risk score and the chosen threshold. For example, if the threshold for classifying the patient as “Yes, this patient has an infection” is 0.75, then the patient's who have a risk score >0.75 would be classified as having an infection.

3) Calculating model performance (sensitivity and specificity) at the pre-specified threshold.

4) Comparing sensitivity/specificity results to pre-specified accuracy requirements.

If the model provides a risk score, the model may be validated by:

1) Calculating the risk score for each new patient;

2) Calculating model performance (sensitivity and specificity) at every possible threshold;

3) Calculate the area under the receiver operating characteristic (“ROC”) curve; and

4) Comparing area under the ROC results to pre-specified accuracy requirements.

### Further Training and Outcome Expansion

After the model is trained, the model may be improved and expanded upon by processing additional patient datasets prospectively. For example, patients with an intelligent implant may be followed forward in time for a number of different clinical outcomes (loosening of implant or micromotion, instability of implant, stiffness and infection, revision surgery, healing date), and a number of different movement types (walking with an assisted device such as a cane, walking with pain, walking with a stiff knee, walking with a shuffle, walking with a limited range of motion, walking up steps and time taken to walk up steps, etc.). This data will be processed and feature engineered as described above and used to retrain the classification model. Over time, the classification model can be trained to include additional outcomes, including real-time classification outcomes and predictive outcomes. For example, a model may process a kinematic signal that includes a jump in the middle of their bouts, plus patient data that indicates an age of over 70, plus a walking speed of around 0.5 m/s, to generate a predictive outcome that the patient has a risk score of getting an infection of 0.032 if the risk score is a probability (or a risk score of 3.2 if the risk score is scaled from 0-100). In another example, the model may process kinematic signal indicative of walking up the steps within a threshold time, to generate a real-time outcome that the patient is doing well.

The automated annotation of the kinematic elements, e.g., fiducial points in time-series waveforms, collected post-implantation enables the creation of biomarkers, e.g., kinematic features such as C-I intervals, which, combined with demographic/prognostic factors, facilitate further model-building. The models may be trained to produce risk scores for different clinical outcomes. These risk scores, derived for each patient over time, represent a time-series allowing for the creation of patient recovery trajectory curves (as described later below in this disclosure). In one example, the unique datasets of many TKA patients over time, and their associated kinematic parameters (walking speed, ROM knee, ROM tibia, stride length etc.), and risk scores or other outputs from predictive trained models, may be used to generate percentile scores for each patient. This may be done in appropriate peer groups defined by factors such as age, gender, height, weight, # of weeks post-op, pre-op condition etc. Recovery trajectory curves can be used to identify patients whose recovery is not going well (examples include below average, or below the 25th percentile), and potentially trigger additional office visits, and interventions with supplementary therapies in order for patients at risk to reach full recovery. Models may be built to estimate the extent of stiffness and pain, infection and loosening in order to monitor the patients' experience and facilitate interventions for those patients having bad recovery experiences.

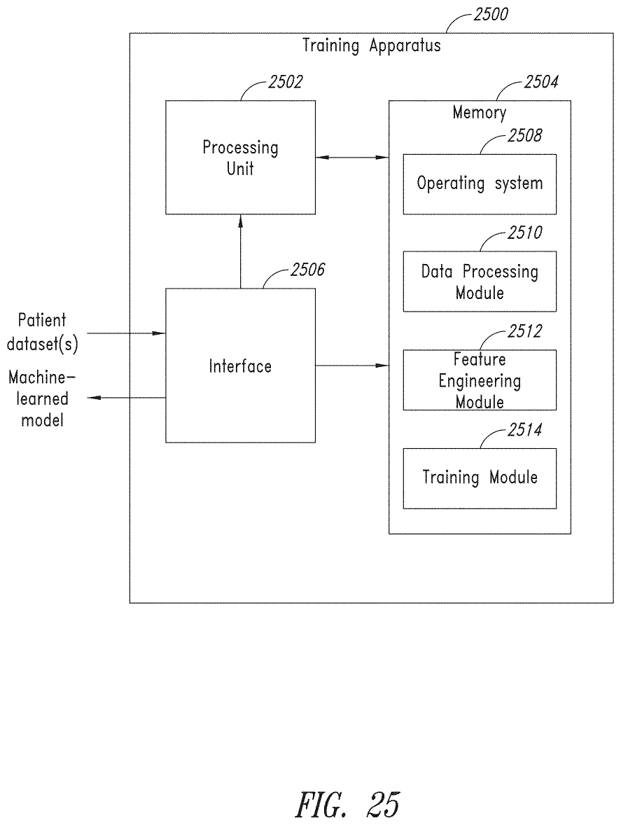

FIG. 25 is a schematic block diagram of an apparatus 2500 corresponding to the training apparatus 1504 of FIG. 16. The apparatus 2500 is configured to execute instructions related to the machine-learned model training processes described above with reference to FIG. 16. The apparatus 2500 may be embodied in any number of processor-driven devices, including, but not limited to, a server computer, a personal computer, one or more networked computing devices, a microcontroller, and/or any other processor-based device and/or combination of devices.

The apparatus 2500 may include one or more processing units 2502 configured to access and execute computer-executable instructions stored in at least one memory 2504. The processing unit 2502 may be implemented as appropriate in hardware, software, firmware, or combinations thereof. A hardware implementation may be a general purpose processor, a graphics processing unit (GPU), a digital signal processor (DSP), an application specific integrated circuit (ASIC), a microprocessor, a microcontroller, a field programmable gate array (FPGA), a System-on-a-Chip (SOC), or any other programmable logic component, discrete gate or transistor logic, discrete hardware components, or any combination thereof, or any other suitable component designed to perform the functions described herein. Software or firmware implementations of the processing unit 2502 may include computer-executable or machine-executable instructions written in any suitable programming language to perform the various functions described herein.

The memory 2504 may include, but is not limited to, random access memory (RAM), flash RAM, magnetic media storage, optical media storage, and so forth. The memory 2504 may include volatile memory configured to store information when supplied with power and/or nonvolatile memory configured to store information even when not supplied with power. The memory 2504 may store various program modules, application programs, and so forth that may include computer-executable instructions that upon execution by the processing unit 2502 may cause various operations to be performed. The memory 2504 may further store a variety of data manipulated and/or generated during execution of computer-executable instructions by the processing unit 2502.

The apparatus 2500 may further include one or more interfaces 2506 that facilitate communication between the apparatus and one or more other apparatuses. For example, the interface 2506 may be configured to receive patient datasets from databases 1514 of the system 1500 of FIG. 15. The interface 2506 is also configured to transmit or send a machine-learned model to other apparatuses, such as a classification apparatus 1506 of the system of FIG. 15. Communication may be implemented using any suitable communications standard. For example, a LAN interface may implement protocols and/or algorithms that comply with various communication standards of the Institute of Electrical and Electronics Engineers (IEEE), such as IEEE 802.11.

The memory 2504 may store various program modules, application programs, and so forth that may include computer-executable instructions that upon execution by the processing unit 2502 may cause various operations to be performed. For example, the memory 2504 may include an operating system module (O/S) 2508 that may be configured to manage hardware resources such as the interface 2506 and provide various services to operations executing on the apparatus 2500.

The memory 2504 stores operation modules such as a data processing module 2510, a feature engineering module 2512, and a training module 2514. These modules may be implemented as appropriate in software or firmware that include computer-executable or machine-executable instructions that when executed by the processing unit 2502 cause various operations to be performed, such as the operations described above with reference to FIG. 16. Alternatively, the modules may be implemented as appropriate in hardware. A hardware implementation may be a general purpose processor, a GPU, a DSP, an ASIC, a FPGA or other programmable logic component, discrete gate or transistor logic, discrete hardware components, or any combination thereof, or any other suitable component designed to perform the functions described herein.

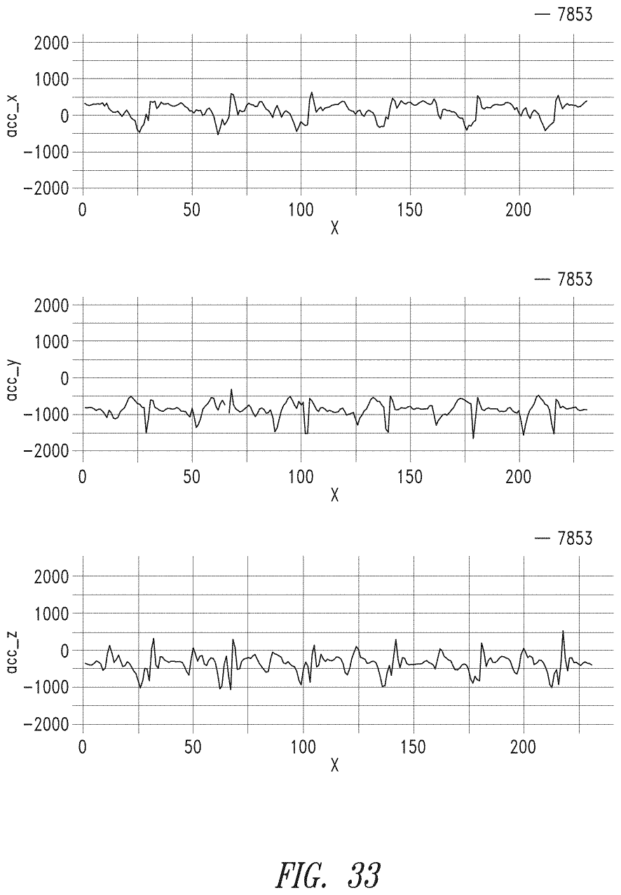

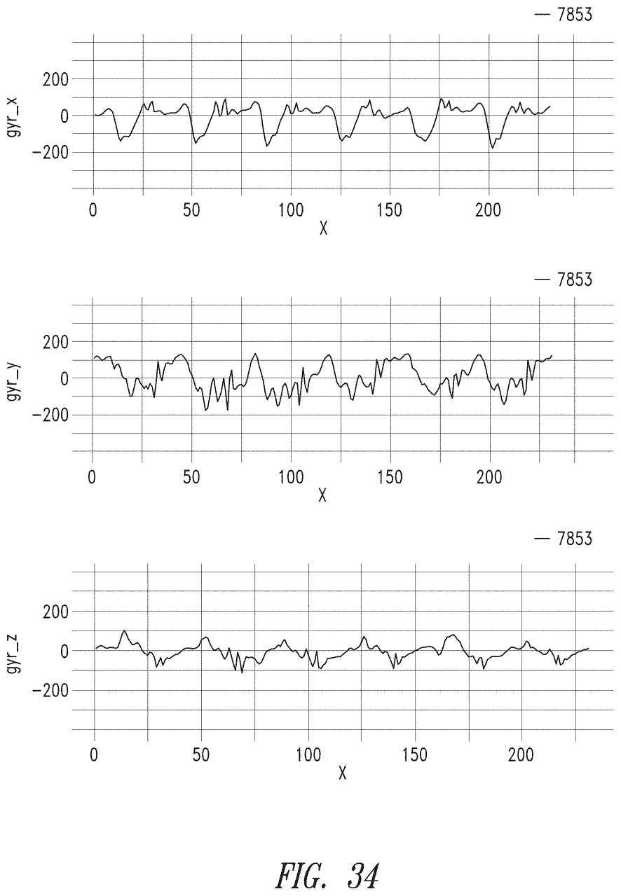

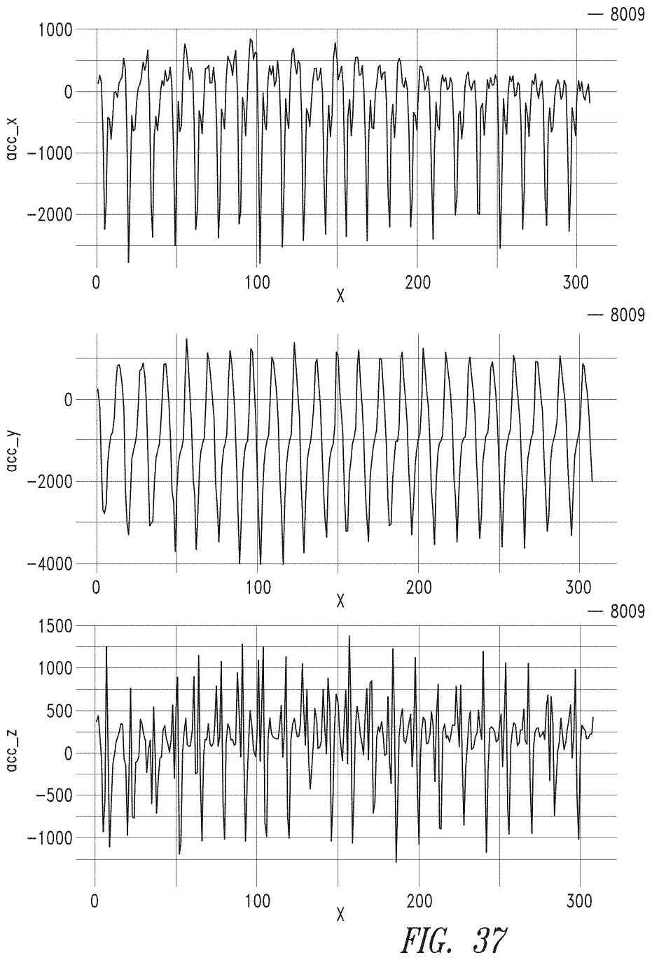

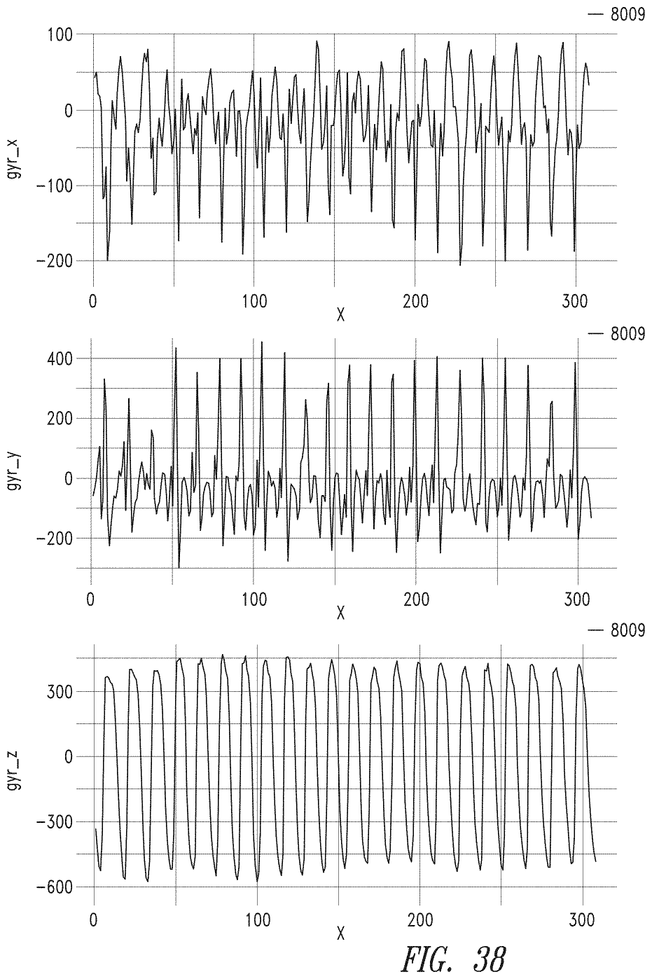

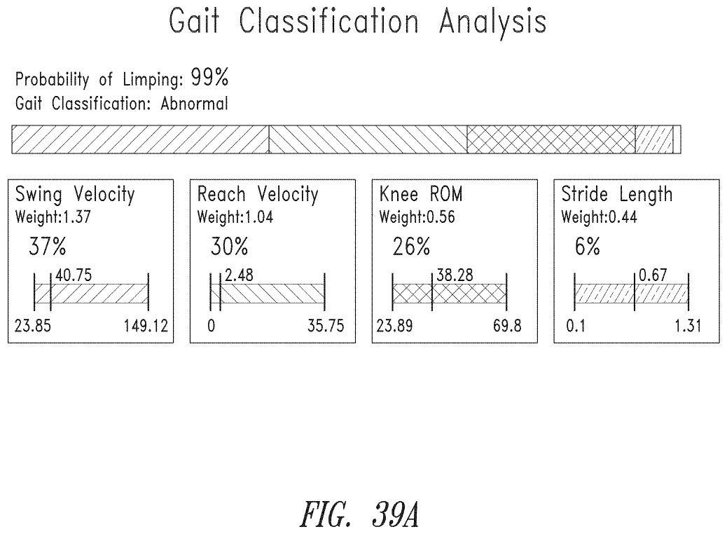

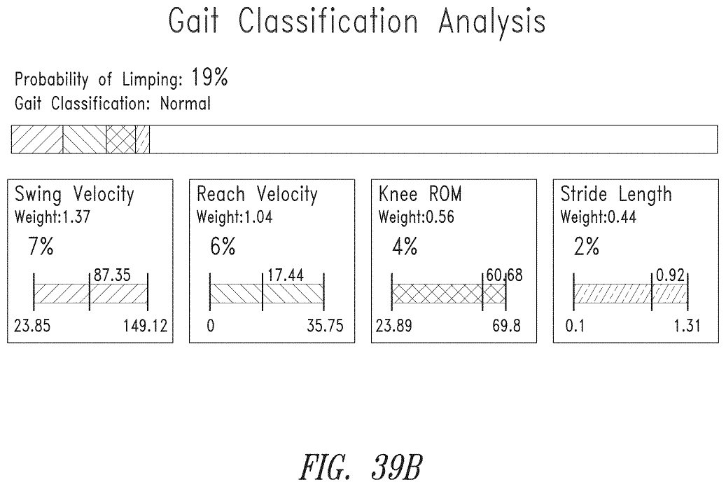

While the preceding description has focused on processing of kinematic data from a sensor associated with a tibia, similar processing may be done with kinematic data sensed by sensors associated with other body parts. For example, with reference to FIGS. 33 and 34, kinematic data sensed by a sensor associated with a hip may be processed to extract features to train a machine-learning model to classify different types of hip movement. Similarly, with reference to FIGS. 37 and 38, kinematic data sensed by a sensor associated with a shoulder may be processed to extract features to train a machine-learning model to classify different types of shoulder movement.

### Classification Apparatus

With reference to FIG. 20, in some embodiments a classification apparatus 1506 for classifying a movement of a body part includes a data processing module 2002, a feature engineering module 2004, and a movement classification model 2008. As described above, a classification apparatus may be configured to classify more than movement of a body part. For example, a classification apparatus may be configured to provide other outcomes. For example, classification model 2008 or outcome model may be trained to provide other diagnostic or prognostic outcomes such as risk of infection, or implant loosening.

The classification apparatus 1506 obtains a dataset 2010 for a subject patient. The subject patient dataset 2010, which may be obtained from the database 1516 of the system of FIG. 15 or directly from the intelligent implant, includes records of motion activity of a body part of the subject patient. For example, the body part may be a tibia and the motion activity may involve movement of the tibia. A subject patient dataset 2010 may include other information, such as: patient demographic data 2020; patient medical data 2022; device operation data 2024; clinical outcome data 2026; clinical movement data 2028; and non-kinematic data 2029.

The classification apparatus 1506 processes the records of motion activity and generates information to which the movement classification model 2008 may be applied. To this end, in some embodiments, the data processing module 2002 receives a record of motion activity comprising raw kinematic data 2012 corresponding to movement of the body part.

The data processing module 2002 processes the raw kinematic data 2012 to provide processed kinematic data 2014. The data processing module 2002 may include the same modules as the data processing module 1602 and may process the raw kinematic data 2012 is the same way as described above with reference to FIGS. 16A and 16B. To this end, the data processing module 2002 may provide processed kinematic data 2014 in the form of one or more of time-series data, fused time-series data, and frequency data.

The feature engineering module 2004 receives the processed kinematic data 2014 in the form of one or more of time-series data, fused time-series data, and frequency data and processes the data to provide kinematic features 2016. The feature engineering module 2004 may include the same modules as the feature engineering module 1604 and may process the processed kinematic data 2014 is the same way as described above with reference to FIGS. 16A, 16B, and 16C. To this end, the feature engineering module 2004 provides kinematic features in the form of one or more of time-series variables, time-series waveforms (individual or fused), spectral variables, and spectral graphs (individual or fused). Note that in the case of a classification model that is trained using deep learning techniques, processed kinematic data 2014 may be input directly to the classification model without being subjected to feature engineering.

The movement classification model 2006 is applied to the one or more kinematic features 2016 to classify the motion activity of the body part as a type of movement. In one configuration, the movement classification model 2006 is a machine-learned algorithm trained in accordance with the process of FIGS. 16A-16D to classify the motion activity of the body part as a type of movement from one or more kinematic features 2016. For example, the body part may be a tibia and the associated movement type may be a normal movement, e.g., walking or running, or an abnormal movement type, e.g., walking with a limp, walking with a limited range of motion, running with a limp, running with a limited range of motion. In other embodiments, if so trained, the classification model 2006 may provide other types of diagnostic or prognostic outcomes such as risk of infection, or implant loosening, or likelihood of full recovery. These outcomes may be quantified in terms of a percentage or scale value (e.g., on a scale of 1 to 10, a patient's level of risk of infection is x)

In some embodiments, the movement classification model 2006 may be applied to the kinematic features 2016 together with other data in the subject patient dataset 2010. For example, the movement classification model 2008 may be applied other data including one or more of patient demographic data 2020; patient medical data 2022; device operation data 2024; clinical outcome data 2026; clinical movement data 2028; and non-kinematic data 2029.

With reference to FIGS. 39A and 39B, in one example embodiment, the classification apparatus 1506 derives a set of kinematic features including swing velocity (peak-to-peak elevation between points C and I), reach velocity (difference in elevation between points C and P), knee ROM and stride length. The measures of these kinematic features may be averaged over a period of time that includes a number of bouts. For example, the period of time may be 24 hours. The movement classification model 2006 may be applied to these four kinematic features alone to provide a movement classification together with a quantification of such classification. The movement classification and quantification may be based on respective individual quantifications derived from each of the four kinematic features. Each individual quantification may correspond to a placement (percentile) of the kinematic features within a range of expected values. For example, with reference to FIG. 39A, swing velocity has a quantification of 37%. Each of the individual quantification may be weighted. For example, continuing with FIG. 39A, the swing velocity quantification has a weight of 1.37. The final movement classification quantification, e.g., abnormal movement in FIG. 39A verses normal movement in FIG. 39B, is derived from the four individual quantifications and their respective weights.

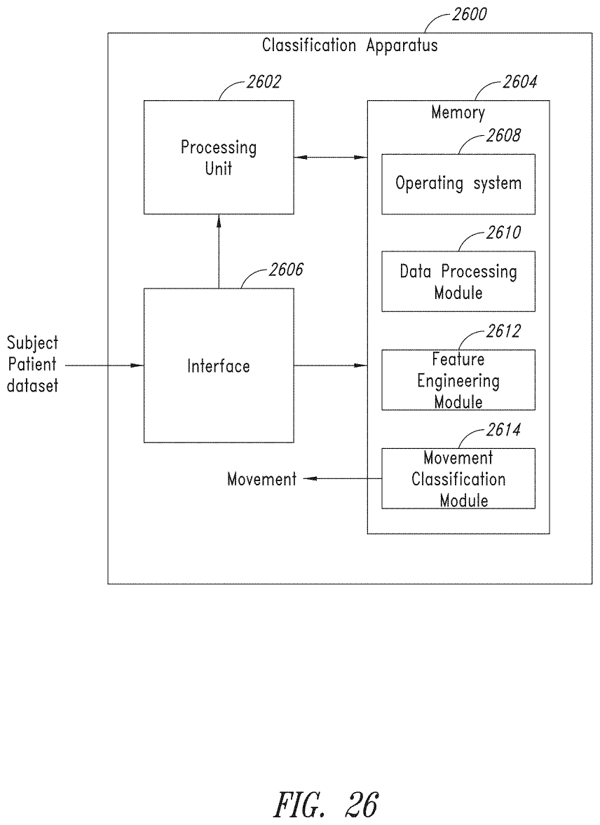

FIG. 26 is a schematic block diagram of an apparatus 2600 corresponding to the classification apparatus 1506 of FIG. 20. The apparatus 2600 is configured to execute instructions related to the machine-learned model training processes described above with reference to FIG. 20. The apparatus 2600 may be embodied in any number of processor-driven devices, including, but not limited to, a server computer, a personal computer, one or more networked computing devices, a microcontroller, and/or any other processor-based device and/or combination of devices.

The apparatus 2600 may include one or more processing units 2602 configured to access and execute computer-executable instructions stored in at least one memory 2604. The processing unit 2602 may be implemented as appropriate in hardware, software, firmware, or combinations thereof. A hardware implementation may be a general purpose processor, graphics processing unit (GPU), a digital signal processor (DSP), an application specific integrated circuit (ASIC), a microprocessor, a microcontroller, a field programmable gate array (FPGA), a System-on-a-Chip (SOC), or any other programmable logic component, discrete gate or transistor logic, discrete hardware components, or any combination thereof, or any other suitable component designed to perform the functions described herein. Software or firmware implementations of the processing unit 2602 may include computer-executable or machine-executable instructions written in any suitable programming language to perform the various functions described herein.

The memory 2604 may include, but is not limited to, random access memory (RAM), flash RAM, magnetic media storage, optical media storage, and so forth. The memory 2604 may include volatile memory configured to store information when supplied with power and/or nonvolatile memory configured to store information even when not supplied with power. The memory 2604 may store various program modules, application programs, and so forth that may include computer-executable instructions that upon execution by the processing unit 2602 may cause various operations to be performed. The memory 2604 may further store a variety of data manipulated and/or generated during execution of computer-executable instructions by the processing unit 2602.

The apparatus 2600 may further include one or more interfaces 2606 that facilitate communication between the apparatus and one or more other apparatuses. For example, the interface 2606 may be configured to receive a subject patient dataset from a database 1514 of the system 1500 of FIG. 15. Communication may be implemented using any suitable communications standard. For example, a LAN interface may implement protocols and/or algorithms that comply with various communication standards of the Institute of Electrical and Electronics Engineers (IEEE), such as IEEE 802.11.

The memory 2604 may store various program modules, application programs, and so forth that may include computer-executable instructions that upon execution by the processing unit 2602 may cause various operations to be performed. For example, the memory 2604 may include an operating system module (O/S) 2608 that may be configured to manage hardware resources such as the interface 2606 and provide various services to operations executing on the apparatus 2600.

The memory 2604 stores operation modules such as a data processing module 2610, a feature engineering module 2612, and a movement classification module 2614. These modules may be implemented as appropriate in software or firmware that include computer-executable or machine-executable instructions that when executed by the processing unit 2602 cause various operations to be performed, such as the operations described above with reference to FIG. 20. Alternatively, the modules may be implemented as appropriate in hardware. A hardware implementation may be a general purpose processor, a GPU, a DSP, an ASIC, a FPGA or other programmable logic component, discrete gate or transistor logic, discrete hardware components, or any combination thereof, or any other suitable component designed to perform the functions described herein.

### Benchmarking Apparatus

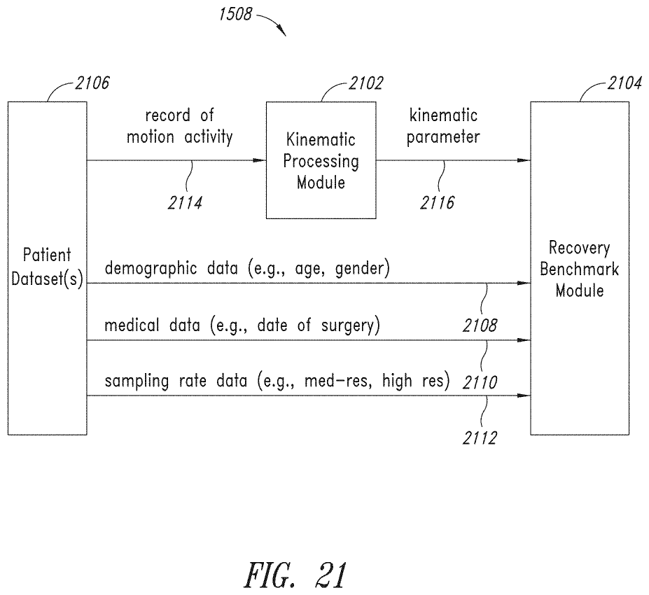

FIG. 21 is a benchmarking apparatus 1508 for generating a benchmark module that provides information for tracking the recovery of a subject patient relative to a similar patient population or for tracking of the condition of a surgical implant. In some embodiments, the tracking standard apparatus 1508 provides information relevant to patients that have undergone a same type of surgery intended to improve the patient motion. For example, the same surgery may be a total knee arthroplasty (TKA). The benchmarking apparatus 1508 includes a kinematic parameter module 2102 and a recovery benchmark module 2104.

The benchmarking apparatus 1508 obtains a number of patient datasets 2106 from across a patient population. Each patient dataset 2106 is associated with a particular patient and includes one or more records of motion activity of a body part of that patient that has undergone surgery. For example, the body part may be a tibia and the motion activity may be movement of the tibia. These records include a time stamp that reflects the time the record was recorded by a sensor. The datasets 2106 may also include patient demographic data 2108 (e.g., age, sex, etc.), patient medical data 2110 (date of surgery, type of surgery, type of implanted device), device operation data 2112 (sampling rate data), clinical outcome data (not shown), clinical movement data (not shown), and/or non-kinematic data (not shown).

For each of a number of records of motion activity in the collection of patient datasets 2106, the kinematic parameter module 2102 calculates a measure of a kinematic parameter 2116 based on the record of motion activity 2114 and provides the kinematic parameter 2116 to the recovery benchmark module 2104. The kinematic parameter 2116 may be, for example, cadence, stride length, walking speed, tibia range of motion, knee range of motion, step count and distance traveled. The kinematic parameter 2116 may be related to the implant state or condition. For example, the kinematic parameter 2116 may be a measure of micromotion of the implant.

For each kinematic parameter 2116, the recovery benchmark module 2104 processes the kinematic parameter, together with its corresponding demographic data 2108, medical data 2110, and sampling-rate data 2112 to derive a benchmark set of information. Each benchmark set of information may include, for example, the value of the kinematic parameter 2116, the time since surgery, the age and sex of the patient, and the sampling rate at which the sensor sensed the motion activity of the record. Regarding the time since surgery, it is calculated based on the time stamp of the record and the time of surgery included in the medical data 2110. Regarding the sampling rate, as previously mentioned, motion activity sensed at a medium resolution by the sensor is relevant to kinematic parameters of the patient, while motion activity sensed at a high resolution by the sensor is relevant to device state.

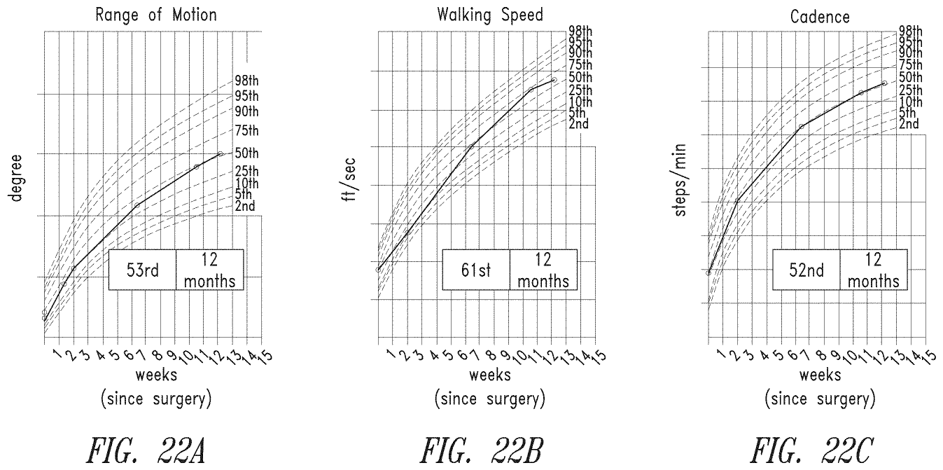

After each of the number of records of motion activity 2114 is processed to obtain a benchmark set of information, the recovery benchmark module 2104 establishes a benchmark dataset against which a subject patient may be compared to track patient recovery or to track implant condition. To this end, the benchmark dataset may be a collection of the benchmark sets of information that may be used to convey different patient-recovery tracks or implant-condition tracks as a function of time. For example, with reference to FIGS. 22A, 22B, and 22C, a benchmark dataset may provide information that enables the creation of a set of percentile curves (light lines) that plot a kinematic parameter as a function of time since surgery. In FIG. 22A, the kinematic parameter is range of motion. In FIG. 22B, the kinematic parameter is walking speed. In FIG. 22C, the kinematic parameter is cadence. The patient-recovery tracks or implant-condition tracks conveyed based on the benchmark dataset may be further refined and filtered based on other information in the benchmark sets of information included in the dataset. For example, the information used to create the percentile curves may be filtered based on demographics to include only information for patients of a specified age or sex. The information used to create the percentile curves may be filtered based on medical data to include only information for patients having a specified medical device.

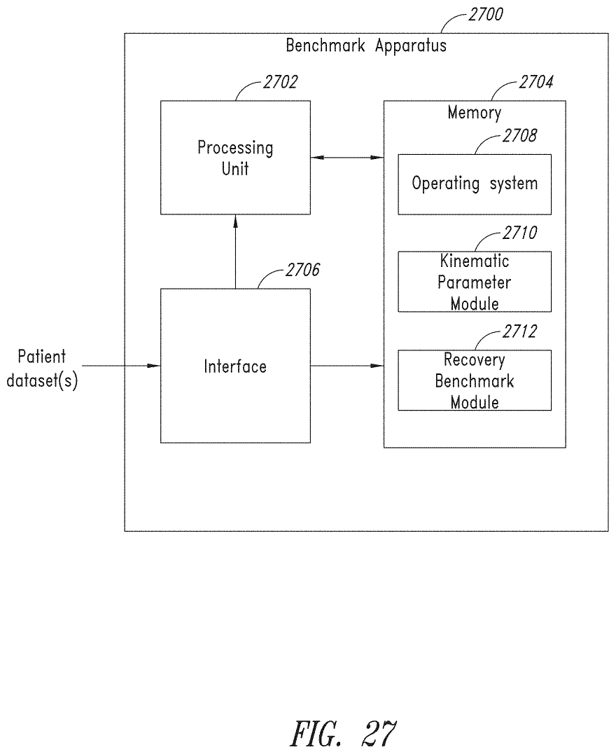

FIG. 27 is a schematic block diagram of an apparatus 2700 corresponding to the benchmarking apparatus 1508 of FIG. 21. The apparatus 2700 is configured to execute instructions related to the machine-learned model training processes described above with reference to FIG. 21. The apparatus 2700 may be embodied in any number of processor-driven devices, including, but not limited to, a server computer, a personal computer, one or more networked computing devices, a microcontroller, and/or any other processor-based device and/or combination of devices.

The apparatus 2700 may include one or more processing units 2702 configured to access and execute computer-executable instructions stored in at least one memory 2704. The processing unit 2702 may be implemented as appropriate in hardware, software, firmware, or combinations thereof. A hardware implementation may be a general purpose processor, a graphics processing unit (GPU), a digital signal processor (DSP), an application specific integrated circuit (ASIC), a microprocessor, a microcontroller, a field programmable gate array (FPGA), a System-on-a-Chip (SOC), or any other programmable logic component, discrete gate or transistor logic, discrete hardware components, or any combination thereof, or any other suitable component designed to perform the functions described herein. Software or firmware implementations of the processing unit 2702 may include computer-executable or machine-executable instructions written in any suitable programming language to perform the various functions described herein.

The memory 2704 may include, but is not limited to, random access memory (RAM), flash RAM, magnetic media storage, optical media storage, and so forth. The memory 2704 may include volatile memory configured to store information when supplied with power and/or nonvolatile memory configured to store information even when not supplied with power. The memory 2704 may store various program modules, application programs, and so forth that may include computer-executable instructions that upon execution by the processing unit 2702 may cause various operations to be performed. The memory 2704 may further store a variety of data manipulated and/or generated during execution of computer-executable instructions by the processing unit 2702.

The apparatus 2700 may further include one or more interfaces 2706 that facilitate communication between the apparatus and one or more other apparatuses. For example, the interface 2706 may be configured to receive patient datasets from a database 1514 of the system 1500 of FIG. 15. Communication may be implemented using any suitable communications standard. For example, a LAN interface may implement protocols and/or algorithms that comply with various communication standards of the Institute of Electrical and Electronics Engineers (IEEE), such as IEEE 802.11.

The memory 2704 may store various program modules, application programs, and so forth that may include computer-executable instructions that upon execution by the processing unit 2702 may cause various operations to be performed. For example, the memory 2704 may include an operating system module (O/S) 2708 that may be configured to manage hardware resources such as the interface 2706 and provide various services to operations executing on the apparatus 2700.

The memory 2704 stores operation modules such as a kinematic parameter module 2710 and a recovery benchmark module 2712. These modules may be implemented as appropriate in software or firmware that include computer-executable or machine-executable instructions that when executed by the processing unit 2702 cause various operations to be performed, such as the operations described above with reference to FIG. 21. Alternatively, the modules may be implemented as appropriate in hardware. A hardware implementation may be a general purpose processor, a GPU, a DSP, an ASIC, a FPGA or other programmable logic component, discrete gate or transistor logic, discrete hardware components, or any combination thereof, or any other suitable component designed to perform the functions described herein.

### Tracking Apparatus

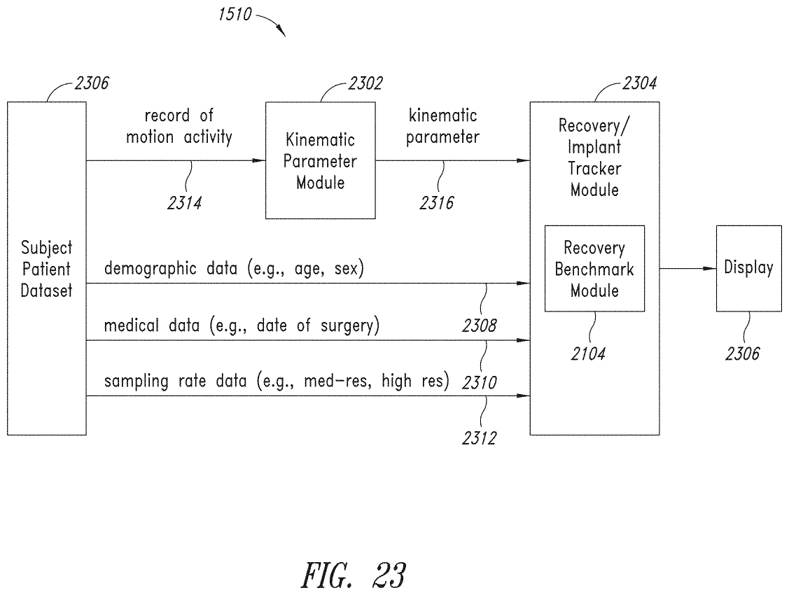

FIG. 23 is a tracking apparatus 1510 for tracking patient recovery or implant state relative to a similar patient population. The tracking apparatus 1510 includes a kinematic parameter module 2302, a recovery/implant tracker module 2304, and a display 2306.

The tracking apparatus 1510 obtains a dataset 2306 from a subject patient population. The dataset 2306 includes one or more records of motion activity of a body part of the patient that has undergone surgery. For example, the body part may be a tibia and the motion activity may be movement of the tibia. These records include a time stamp that reflects the time the record was recorded by a sensor. The datasets 2306 may also include patient demographic data 2308 (e.g., age, sex, etc.), patient medical data 2310 (date of surgery, type of surgery, type of implanted device), device operation data 2312 (sampling rate data), clinical outcome data (not shown), clinical movement data (not shown), and/or non-kinematic data (not shown).

For an individual record of motion activity in the dataset 2306, the kinematic parameter module 2302 calculates a measure of a kinematic parameter 2316 based on the record of motion activity 2314 and provides the kinematic parameter 2316 to the recovery/implant tracker module. The kinematic parameter 2316 may be, for example, range of motion, walking speed, cadence, limp severity.

For an individual kinematic parameter 2116, the recovery/implant tracker module 2304 processes the kinematic parameter, together with its corresponding demographic data 2308, medical data 2310, and sampling-rate data 2312 to derive a set of information. The set of information may include, for example, the value of the kinematic parameter 2316, the time since surgery, the age and sex of the patient, and the sampling rate at which the sensor sensed the motion activity of the record. Regarding the time since surgery, it is calculated based on the time stamp of the record and the time of surgery included in the medical data 2310. Regarding the sampling rate, as previously mentioned, motion activity sensed at a medium resolution by the sensor is relevant to kinematic parameters of the patient, while motion activity sensed at a high resolution by the sensor is relevant to device state.

Having processed a sufficient number of individual records of motion activity 2314 for the subject patient, the recovery/implant tracker module 2304 establishes a dataset to use in comparison with a benchmark dataset provided by the recovery benchmark module 2104 to determine a patient recovery state or an implant device state. For example, with reference to FIGS. 22A, 22B, and 22C, a subject patient dataset may provide information that enables the creation of a subject patient curve that overlays a set of percentile curves enabled by the benchmark dataset provided by the recovery benchmark module 2104. The recovery/implant tracker module 2304 may output a signal to a display 2306 that enables a visual display like those shown in FIGS. 22A, 22B, and 22C.

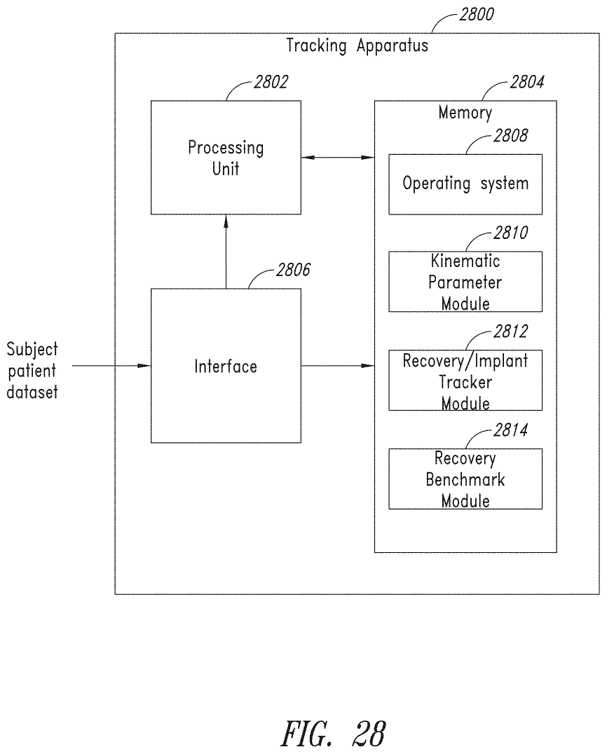

FIG. 28 is a schematic block diagram of an apparatus 2800 corresponding to the tracking apparatus 1510 of FIG. 23. The apparatus 2800 is configured to execute instructions related to the machine-learned model training processes described above with reference to FIG. 23. The apparatus 2800 may be embodied in any number of processor-driven devices, including, but not limited to, a server computer, a personal computer, one or more networked computing devices, a microcontroller, and/or any other processor-based device and/or combination of devices.

The apparatus 2800 may include one or more processing units 2802 configured to access and execute computer-executable instructions stored in at least one memory 2804. The processing unit 2802 may be implemented as appropriate in hardware, software, firmware, or combinations thereof. A hardware implementation may be a general purpose processor, a graphics processing unit (GPU), a digital signal processor (DSP), an application specific integrated circuit (ASIC), a microprocessor, a microcontroller, a field programmable gate array (FPGA), a System-on-a-Chip (SOC), or any other programmable logic component, discrete gate or transistor logic, discrete hardware components, or any combination thereof, or any other suitable component designed to perform the functions described herein. Software or firmware implementations of the processing unit 2802 may include computer-executable or machine-executable instructions written in any suitable programming language to perform the various functions described herein.

The memory 2804 may include, but is not limited to, random access memory (RAM), flash RAM, magnetic media storage, optical media storage, and so forth. The memory 2804 may include volatile memory configured to store information when supplied with power and/or nonvolatile memory configured to store information even when not supplied with power. The memory 2804 may store various program modules, application programs, and so forth that may include computer-executable instructions that upon execution by the processing unit 2802 may cause various operations to be performed. The memory 2804 may further store a variety of data manipulated and/or generated during execution of computer-executable instructions by the processing unit 2802.

The apparatus 2800 may further include one or more interfaces 2806 that facilitate communication between the apparatus and one or more other apparatuses. For example, the interface 2806 may be configured to receive a subject patient dataset from a database 1514 of the system 1500 of FIG. 15. Communication may be implemented using any suitable communications standard. For example, a LAN interface may implement protocols and/or algorithms that comply with various communication standards of the Institute of Electrical and Electronics Engineers (IEEE), such as IEEE 802.11.

The memory 2804 may store various program modules, application programs, and so forth that may include computer-executable instructions that upon execution by the processing unit 2802 may cause various operations to be performed. For example, the memory 2804 may include an operating system module (O/S) 2808 that may be configured to manage hardware resources such as the interface 2806 and provide various services to operations executing on the apparatus 2800.

The memory 2804 stores operation modules such as a kinematic parameter module 2810, a recovery/implant tracker module 2812, and a recovery benchmark module 2814. These modules may be implemented as appropriate in software or firmware that include computer-executable or machine-executable instructions that when executed by the processing unit 2802 cause various operations to be performed, such as the operations described above with reference to FIG. 23. Alternatively, the modules may be implemented as appropriate in hardware. A hardware implementation may be a general purpose processor, a GPU, a DSP, an ASIC, a FPGA or other programmable logic component, discrete gate or transistor logic, discrete hardware components, or any combination thereof, or any other suitable component designed to perform the functions described herein.

### Configuration Management Apparatus

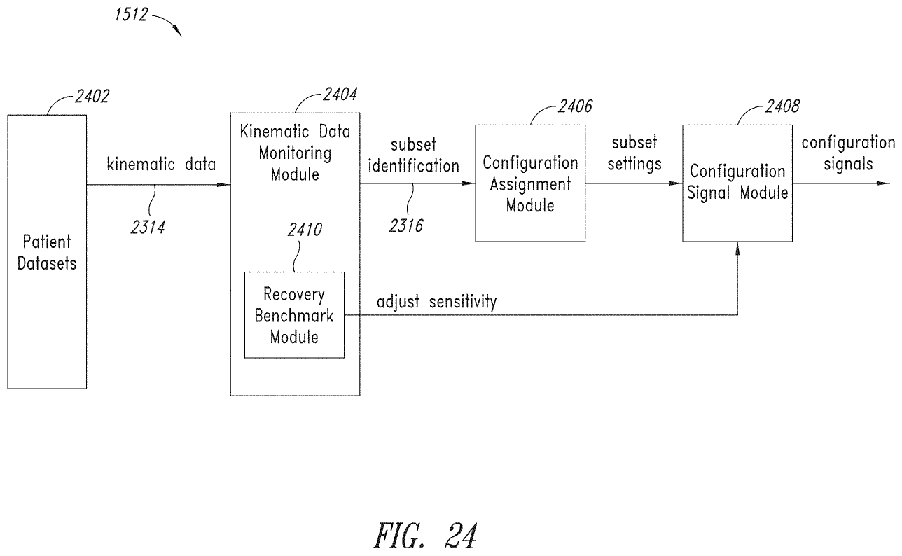

FIG. 24 is a configuration management apparatus 1512 for managing operational parameters of intelligent implants to improve the collection of data by such implants. The configuration management apparatus 1512 includes a kinematic data monitoring module 2404, a configuration assignment module 2406 and a configuration signal module 2408.

The kinematic data monitoring module 2404 obtains kinematic data indicative of patient activity from a number of intelligent implants across a patient population. Each intelligent implant is implanted in a patient, and the kinematic data is obtained from one or more sensors of the intelligent implant. The kinematic data monitoring module 2404 is configured to monitor the obtained kinematic data over time and to separate the patient population into a plurality of subsets of the patient population, where each patient in a subset of the patient population has provided kinematic data indicative of a substantially similar pattern of patient activity during a specified time period, e.g., 24 hours.

To this end, the kinematic data includes, for each of the number of intelligent implants across the patient population, information indicative of the times when a sensor in the implant detects activity at or above a threshold. For example, a sensor may be configured to detect activity corresponding to one of steps by the patient, or significant motion by the patient. Based on this information, the kinematic data monitoring module 2404 determines, for each of the number of intelligent implants, a first time period within the specified time period during which the patient is likely to be active. Based on the first time period, the kinematic data monitoring module 2404 further determines a second time period within the specified time period during which the patient is likely to be inactive. The second time period may be a period of time that is exclusive of the first time period. For example, a first time period may be from 6:00 am to 10:00 pm, in which case the second time period would be 10:00 pm to 6:00 am. The kinematic data monitoring module 2404 determines a first time period and a second time period for each intelligent implants across the patient population and then groups the patients into subsets of the patient population based on their respective first time periods and second time periods.

The configuration assignment module 2406 is configured to assign a data sampling configuration to each subset of the patient population. To this end, the configuration assignment module 2406 generates a data sampling configuration that configures the intelligent implants in each particular subset to sample data from the one or more sensors during the first time period, in accordance with a sampling schedule, and to refrain from sampling data from the one or more sensors during the second time period. The sampling schedule may be defined by start and stop times (e.g., start time=7 am, stop time=10 pm). Different start and stop times may be set for the low-resolution sampling windows, and each of a number of medium-resolution sampling windows. In one implementation there are three separate medium-resolution sampling windows.

The configuration signal module 2408 provides a signal for each respective intelligent implant within a respective subset of the patient population. The signal is configured to set the data sampling configuration of the intelligent implant in accordance with the data sampling assigned to the subset by the configuration assignment module 2406. The signal may be provided directly to the intelligent implant or may be provided to a base station associated with the intelligent implant for subsequent upload to the implant by the base station.

Considering the kinematic data monitoring module 2404 further, in some embodiments the one or more sensors of the intelligent implants are configured to trigger data sampling and recording upon occurrence of a threshold force. In this case, a sensitivity adjustment module 2410 of the kinematic data monitoring module 2404 is configured to identify one or more patients whose associated intelligent implant is failing to provide kinematic data; and to adjust the sensitivity of the one or more sensors to require less force to trigger data sampling and recording. The sensitivity adjustment module 2410 is also further configured to identify one or more patients whose associated intelligent implant provides kinematic data indicative of non-walking activity, e.g., such as moving the knee in bed, swinging the knee on a chair, or getting in and out of a car; and to adjust the sensitivity of the sensor to require more force to trigger data sampling and recording. The sensitivity adjustment module 2410 may adjust sensitivity through the configuration signal module 2408 by providing a sensitivity setting to the configuration signal module, together with an identification of the relevant intelligent implant, and request that the configuration signal module transmit a signal to the implant, or a base station associated with the implant, where the signal is configured to set the sensitivity as indicated by the sensitivity adjustment module 2410.

As previously described detection of a significant motion event is based on a set of programmable parameters including a significant motion threshold, a skip time, and a proof time. A significant motion is a change in acceleration as determined from the samples of one or more of the accelerometers. In one configuration, the default settings for the significant motion threshold is in the range of 2 mg and 4 mg, the default settings for the skip time is in the range of 1.5 seconds to 3.5 seconds, and the default settings for the proof time is in the range of 0.7 seconds and 1.3 seconds. These programmable parameters may be adjusted based on analyses of the number of significant motion events confirmed by an SMU 1022.

For example, if a patient is not triggering the medium-resolution windows (ten second bouts) three times per day as expected, yet the patient is determined to have been walking during the medium-resolution windows (based on the step counts from the low-resolution data), the programmable parameters, e.g., significant motion threshold, skip time, and proof time, are adjusted to better ensure triggering of the medium-resolution windows. These patients may be characterized as light/slow walkers.

The below table summarizes adjustments to the programmable parameters in the case of a light/slow walker.

| | Default | Make it easier to trigger |
|---|---|---|
| Significant motion | 312 mg | Lower this value. By lowering this value, smaller slopes |
| | | (changes in acceleration) are considered significant. So, the |
| | | device will trigger medium-resolution sampling when a person |
| | | is moving slowly. |
| Skip time | 2.56 | Lower this value. By lowering this value, the device will trigger |
| | seconds | medium-resolution sampling even if a person is not walking for |
| | | a long period of time. Say they only walk from a bed to a chair |
| | | 3 steps away. We need to catch them moving twice during |
| | | that period of time and if the skip time is too long, they will be |
| | | in the chair before we get to the proof time. |
| Proof time | 0.96 | Raise this value. By increasing the proof time, the device gives |
| | seconds | the patient more time to have another “significant motion”. If |
| | | the patient gets up to walk, and then take a few seconds to |
| | | move again, the device gives them more time to make that |
| | | second significant motion. |

In another scenario, if medium-resolution sampling is triggered (ten second bouts recorded) but it is determined that the patient was not moving or walking, the programmable parameters are adjusted to make it harder for the device to trigger medium-resolution sampling. This scenario is detected through analysis of IMU sensor signals captured during a medium-resolution sampling bout. If the sensor signals are “flat,” which is indicative of no motion or movement of the sensor, then it is determined that the device was triggered into medium-resolution sampling during a time when the tibia was not moving. Also, if there is a non-flat tracing, but it is not cyclical (meaning, there are not nice, neat, evenly spaced repeated cycles), then it is determined that the device was triggered into medium-resolution sampling during a time when the person was not walking. Instead, the patient may have been turning over in bed, bouncing their knee, or getting out of a car.

It is noted that the sensor signal tracings for walking are very recognizable, and may be automatically detected by one or more computer algorithms, without human supervision. Likewise, a computer algorithm may be configured to automatically detect the above conditions of flat (no motion) and non-flat and non-cyclic.

In order to make it harder to trigger, the programmable parameters are adjusted opposite the way they were adjusted above in the case of a light/slow walker. The below table summarizes adjustments to the programmable parameters in this case.

| | Default | Make it harder to trigger |
|---|---|---|
| Significant motion | 312 mg | Raise this value. By raising this value, bigger slopes (changes in |
| | | acceleration) are necessary. So, we will only catch people who |
| | | are moving quickly. |
| Skip time | 2.56 | Raise this value. By raising this value, we catch people only if |
| | seconds | their motion is persistent—and occurs twice even if those 2 |
| | | occurrences are “far” apart in time. So we wont get them |
| | | walking from the bed to the chair. We will only trigger it if they |
| | | are walking longer distances, say from the bedroom to the |
| | | living room. |
| Proof time | 0.96 | Lower this value. By decreasing the proof time, we give the |
| | seconds | patient LESS time to have another “significant motion”. They |
| | | have to have the 2^{nd} significant motion in a shorter period of |
| | | time, which would require more frequent significant motion |
| | | events. |

The sampling rate and the size of the data collection time window may be adjusted to capture micromotion without unduly compromising battery life. Micromotion can be detected by the accelerometer as high frequency vibrations. To detect such vibrations, the sampling frequency may be at least twice the vibration frequency. Also, the wider the time window, the better the chance to capture micromotion. However, high sampling frequency and wide time window cost battery life. To find the optimal setting, the device is initially programmed to collect three bouts of 10-second data a day at a relatively low frequency of 25 Hz (accelerometers and gyroscopes) and one bout of 3-second data a day at a high frequency of 800 Hz (accelerometer only). The high frequency data is analyzed to detect vibrations below 400 Hz are detected, and if such vibrations are detected, to determine how high in frequency those vibrations can go. Based on this information the sampling frequency and the width of the time window of the other bouts may be adjusted just enough to capture high frequency vibrations without unnecessarily using battery life. This cycle of insight generation to adjustment is automated so that the sampling rate is continually optimized on both power consumption and information value.

In addition, the time recording default settings can also be changed from recording during three set time windows a day, morning, afternoon, evening. Consider a patient who works the overnight shift. Under the default settings the patient may be sleeping during two of the recording windows. Therefore the system monitors the number of default windows that trigger significant motion resulting in the collection of qualified walking motion data. If the system detects that a patient consistently fails to trigger the significant motion threshold, then with that insight the time window settings can be adjusted. This cycle of insight generation to adjustment is automated so that the time windows are optimized for successful data capture.

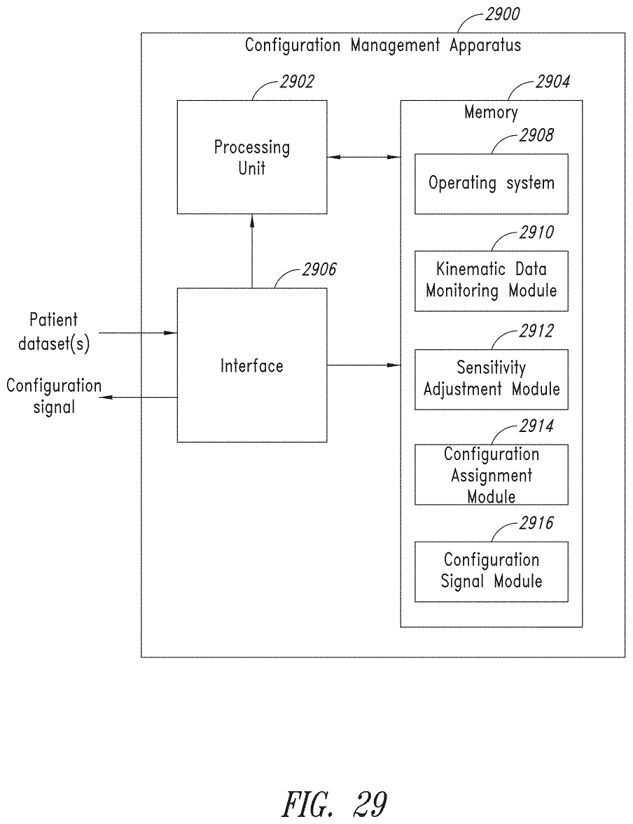

FIG. 29 is a schematic block diagram of an apparatus 2900 corresponding to the configuration management apparatus 1512 of FIG. 24. The apparatus 2900 is configured to execute instructions related to the machine-learned model training processes described above with reference to FIG. 24. The apparatus 2900 may be embodied in any number of processor-driven devices, including, but not limited to, a server computer, a personal computer, one or more networked computing devices, a microcontroller, and/or any other processor-based device and/or combination of devices.

The apparatus 2900 may include one or more processing units 2902 configured to access and execute computer-executable instructions stored in at least one memory 2904. The processing unit 2902 may be implemented as appropriate in hardware, software, firmware, or combinations thereof. A hardware implementation may be a general purpose processor, a digital signal processor (DSP), an application specific integrated circuit (ASIC), a microprocessor, a microcontroller, a field programmable gate array (FPGA), a System-on-a-Chip (SOC), or any other programmable logic component, discrete gate or transistor logic, discrete hardware components, or any combination thereof, or any other suitable component designed to perform the functions described herein. Software or firmware implementations of the processing unit 2902 may include computer-executable or machine-executable instructions written in any suitable programming language to perform the various functions described herein.

The memory 2904 may include, but is not limited to, random access memory (RAM), flash RAM, magnetic media storage, optical media storage, and so forth. The memory 2904 may include volatile memory configured to store information when supplied with power and/or nonvolatile memory configured to store information even when not supplied with power. The memory 2904 may store various program modules, application programs, and so forth that may include computer-executable instructions that upon execution by the processing unit 2902 may cause various operations to be performed. The memory 2904 may further store a variety of data manipulated and/or generated during execution of computer-executable instructions by the processing unit 2902.

The apparatus 2900 may further include one or more interfaces 2906 that facilitate communication between the apparatus and one or more other apparatuses. For example, the interface 2906 may be configured to receive patient datasets from a database 1514 of the system 1500 of FIG. 15. Communication may be implemented using any suitable communications standard. For example, a LAN interface may implement protocols and/or algorithms that comply with various communication standards of the Institute of Electrical and Electronics Engineers (IEEE), such as IEEE 802.11.

The memory 2904 may store various program modules, application programs, and so forth that may include computer-executable instructions that upon execution by the processing unit 2902 may cause various operations to be performed. For example, the memory 2904 may include an operating system module (O/S) 2908 that may be configured to manage hardware resources such as the interface 2906 and provide various services to operations executing on the apparatus 2900.

The memory 2904 stores operation modules such as a kinematic data monitoring module 2910, a sensitivity adjustment module 2912, a configuration assignment module 2914, and a configuration signal module 2916. These modules may be implemented as appropriate in software or firmware that include computer-executable or machine-executable instructions that when executed by the processing unit 2902 cause various operations to be performed, such as the operations described above with reference to FIG. 24. Alternatively, the modules may be implemented as appropriate in hardware. A hardware implementation may be a general purpose processor, a DSP, an ASIC, a FPGA or other programmable logic component, discrete gate or transistor logic, discrete hardware components, or any combination thereof, or any other suitable component designed to perform the functions described herein.

Some inventive aspects of the disclosure are set forth in the following clauses:

Clause 1. A method of generating a machine-learned classification model, the method comprising:

obtaining a plurality of records from across a patient population, each of the plurality of records including kinematic data corresponding to motion activity of a body part;

for each record:
identifying elements in the kinematic data,
deriving one or more kinematic features based on the elements, and
labeling the record and each of the one or more kinematic features with a movement type; and

training a machine-learned model on the labeled kinematic features to classify movement of a body part as a particular movement type.

Clause 2. The method of clause 1, wherein identifying elements in the kinematic data comprises:

representing the kinematic data as a time-series waveform, and

identifying a set of fiducial points in the time-series waveform, the set of points corresponding to the elements.

Clause 3. The method of clause 2, wherein movement of the body part corresponds to a gait cycle, and the elements correspond to points in the gait cycle that correspond to one of a heel-strike, a loading response, a mid-stance, a terminal stance, a pre-swing, a toe-off, a mid-swing, and a terminal swing.

Clause 4. The method of any one of clauses 2 and 3, wherein identifying a set of fiducial points comprises applying a feature extraction algorithm to the time-series waveform to automatically detect the points.

Clause 5. The method of clause 1, wherein identifying elements in the record comprises:

representing the kinematic data as a spectral distribution graph, and

identifying a set of peaks in the spectral distribution graph, the set of peaks corresponding to the elements.

Clause 6. The method of any one of clauses 1-5, wherein labeling the record and each of the one or more kinematic features with a movement type comprises:

representing each kinematic data included in the plurality of records as one of a time-series waveform or a spectral distribution graph, and

applying a clustering algorithm to the plurality of time-series waveforms or spectral distribution graphs that automatically separates the plurality of time-series waveforms or spectral distribution graphs into a plurality of clusters based on similarities.

Clause 6a. The method of clause 6, wherein the clustering algorithm automatically assigns a movement type to one or more of the plurality of clusters, which movement type is also assigned to each of the time-series waveforms or the spectral distribution graphs within the cluster.

Clause 7. The method of clause 1, wherein the particular movement type comprises one of a normal movement type and an abnormal movement type.

Clause 8. The method of clause 1, wherein the body part comprises a boney structure.

Clause 9. The method of clause 8, wherein the boney structure is associated with a body joint comprising one of a hip joint, knee joint, ankle joint, shoulder joint, elbow joint, and wrist joint.

Clause 10. The method of clause 1, wherein the records are obtained from a sensor associated with the body part.

Clause 11. The method of clause 10, wherein the sensor is an external sensor.

Clause 12. The method of clause 10, wherein the sensor is an implanted sensor.

Clause 13. The method of clause 12, wherein the implanted sensor is implanted within the body part.

Clause 14. The method of clause 13, wherein the body part is a boney structure.

Clause 15. The method of clause 10, wherein the sensor comprises a gyroscope oriented relative to the body part and configured to provide as kinematic data, a signal corresponding to angular velocity about a first axis relative to the body part.

Clause 16. The method of clause 10, wherein the sensor comprises an accelerometer oriented relative to the body part and configured to provide as kinematic data, a signal corresponding to acceleration along a first axis relative to the body part.

Clause 17. The method of either of clause 15 or 16, wherein the first axis is one of three axes of a three-dimensional coordinate system.

Clause 18. The method of either of clause 15 or 16, wherein the first axis is one axis of a coordinate system comprising a second axis, and obtaining records of motion activity further comprises:

obtaining from the sensor, as kinematic data, a signal corresponding to angular velocity about the second axis relative to the body part, and/or a signal corresponding to acceleration along the second axis relative to the body part.

Clause 19. The method of clause 18, wherein the first axis and the second axis are axes of a three-dimensional coordinate system further comprising a third axis, and obtaining records of motion activity further comprises:

obtaining from the sensor, as kinematic data, a signal corresponding to angular velocity about the third axis relative to the body part, and/or a signal corresponding to acceleration along the third axis relative to the body part.

Clause 20. The method of clauses 18 or 19, further comprising, prior to labeling the records:

combining two or more of the respective signals of angular velocity about the first axis, the second axis, and the third axis; and/or

combining two or more of the respective signals of acceleration along the first axis, the second axis, and the third axis.

Clause 21. The method of clause 20, further comprising combining all respective signals.

Clause 22. The method of clause 1, wherein the plurality of records further comprises one or more of patient demographic data, patient medical data, device operation data, clinical outcome data, clinical movement data, non-kinematic data, unsupervised labels, and supervised labels, and training further comprises training the machine-learned model on the labeled kinematic features and their corresponding additional data.

Clause 23. A computer-implemented method comprising:

obtaining a plurality of records from across a patient population, each of the plurality of records including kinematic data corresponding to motion activity of a body part;

for each record:
identifying elements in the kinematic data,
deriving one or more kinematic features based on the elements, and
labeling the record and each of the one or more kinematic features with a movement type; and

training a machine-learned model on the labeled kinematic features to classify movement of a body part as a particular movement type.

Clause 24. The computer-implemented method of clause 23, further comprising the methods of any one of clauses 2-22.

Clause 25. A training apparatus comprising:

a memory; and

a processor coupled to the memory and configured to:
obtain a plurality of records from across a patient population, each of the plurality of records including kinematic data corresponding to motion activity of a body part;
for each record:
   identify elements in the kinematic data,
   derive one or more kinematic features based on the elements, and
   label the record and each of the one or more kinematic features with a movement type; and
train a machine-learned model on the labeled kinematic features to classify movement of a body part as a particular movement type.

Clause 26. The training apparatus of clause 25, wherein the processor is further configured to implement the methods of any one of clauses 2-22.

Clause 27. A method comprising:

obtaining a record including kinematic data corresponding to motion activity of a body part of a patient; and

applying a machine-learned classification model to the kinematic data or to one or more kinematic features derived from the kinematic data to classify the motion activity of the body part as a type of movement.

Clause 28. The method of clause 27, wherein the machine-learned classification model is trained in accordance with one or more of clause 1-21.

Clause 29. The method of clause 27, wherein applying a machine-learned classification model comprises:

identifying elements in the kinematic data;

deriving the one or more kinematic features based on the elements; and

applying the machine-learned model to the one or more kinematic features.

Clause 29a. The method of clause 27, wherein applying a machine-learned classification model comprises:

generating a visual representation of the kinematic data; applying the machine-learned model to the visual representation.

Clause 29b. The method of clause 29a, wherein the visual representation comprises one of a of a time-series waveform or a spectral distribution graph.

Clause 30. The method of any one of clause 27-29b, wherein the method is implemented by a computer.

Clause 31. A classification apparatus comprising:

a memory; and

a processor coupled to the memory and configured to:
obtain a record including kinematic data corresponding to motion activity of a body part of a patient; and
apply a machine-learned classification model to the kinematic data or to one or more kinematic features derived from the kinematic data to classify the motion activity of the body part as a type of movement.

Clause 32. The classification apparatus of clause 31, wherein the processor is further configured to implement the methods of any one of clauses 28-30.

Clause 33. A method comprising: obtaining kinematic data from a sensor implanted in a bone associated with a joint; and assessing movement of the joint based on a representation of the kinematic data.

Clause 34. The method of clause 33, wherein the representation is a time-series waveform.

Clause 35. The method of clause 33, wherein the representation is a spectral distribution graph.

Clause 36. The method of clause 33, wherein assessing movement comprises determining a movement type for the movement of the joint.

Clause 37. The method of clause 36, wherein determining a movement type comprises applying a machine-learned algorithm to the representation to classify the movement of the joint as a particular type of movement.

Clause 38. The method of clause 36, wherein determining a movement type comprises:

identifying elements in the representation;

deriving one or more kinematic features based on the elements; and

applying a machine-learned model to the one or more kinematic features to classify the movement of the joint as a particular type of movement.

Clause 39. The method of clause 33, wherein assessing movement comprises:

determining a biomarker from the kinematic data;

comparing the biomarker to a baseline biomarker; and

determining a patient recovery state based on a comparison outcome.

Clause 40. The method of clause 39, wherein the biomarker comprises one of a kinematic feature derived from a time-series representation or a spectral distribution representation of the kinematic data, or a kinematic parameter derived based on acceleration and angular velocity measurements included in the kinematic data.

Clause 40a. The method of clause 40, wherein the kinematic feature comprises one of time intervals between elements, ratios based on one or more of the intervals, elevation (or offset) of a kinematic feature relative to a reference line, and elevation difference between different elements.

Clause 40b. The method of clause 40, wherein the kinematic parameter comprises one or more of cadence, stride length, walking speed, tibia range of motion, knee range of motion, step count and distance traveled.

Clause 41. The method of clause 39, wherein:

a patient recovery state comprises an improved state when/if the biomarker is greater than the baseline biomarker.

Clause 42. The method of clause 39, wherein:

a patient recovery state comprises an improved state when/if the biomarker is less than the baseline biomarker.

Clause 43. The method of clause 39, wherein the baseline biomarker is derived from previously obtained kinematic data from the sensor implanted in the bone associated with the joint.

Clause 44. The method of clause 39, wherein the baseline biomarker is derived from kinematic data obtained from a plurality of other sensors of the same type as the sensor, wherein the other sensors are implanted in a bone associated with a joint that are the same type of bone and joint of the joint.

Clause 45. The method of any one of clause 33-44, wherein the method is implemented by a computer.

Clause 46. A classification apparatus comprising:

a memory; and

a processor coupled to the memory and configured to:
obtain kinematic data from a sensor implanted in a bone associated with a joint; and
assess movement of the joint based on a representation of the kinematic data.

Clause 47. The classification apparatus of clause 46, wherein the processor is further configured to implement the methods of any one of clauses 34-45.

Clause 48. A method comprising:

obtaining a representation of movement of a body part of a patient;

deriving one or more biomarkers from the representation; and

classifying the movement of the body part as normal movement or abnormal movement based on the one or more biomarkers.

Clause 49. The method of clause 48, wherein the body part comprises a boney structure.

Clause 50. The method of clause 49, wherein the boney structure is associated with a body joint comprising one of a hip joint, knee joint, ankle joint, shoulder joint, elbow joint, and wrist joint.

Clause 51. The method of clause 48, wherein obtaining a representation of movement of a body part comprises receiving a record of kinematic data from a sensor associated with the body part.

Clause 52. The method of clause 51, wherein the sensor is an external sensor.

Clause 53. The method of clause 51, wherein the sensor is an implanted sensor.

Clause 54. The method of clause 53, wherein the implanted sensor is implanted within the body part.

Clause 55. The method of clause 54, wherein the body part is a boney structure.

Clause 56. The method of clause 51, wherein the sensor comprises a gyroscope oriented relative to the body part and configured to provide as kinematic data, a signal corresponding to angular velocity about an axis relative to the body part.

Clause 57. The method of clause 51, wherein the sensor comprises an accelerometer oriented relative to the body part and configured to provide as kinematic data, a signal corresponding to acceleration along an axis relative to the body part.

Clause 58. The method of clause 48, wherein the representation corresponds to a cyclic time-series waveform, and deriving one or more metrics from the representation comprises:

identifying elements of the cyclic time-series waveform; and

calculating the one or more biomarkers based on one or more of the identified elements.

Clause 59. The method of clause 58, wherein the identified elements correspond to different points in the cyclic time-series waveform and the calculated one or more biomarkers comprise one or more of a time interval between pairs of points, ratios of time intervals between pairs of points, elevations of points relative to a baseline of the time-series waveform, differences in elevations between a pair of points.

Clause 59a. The method of clause 48, wherein the representation corresponds to a spectral distribution graph, and deriving one or more metrics from the representation comprises:

identifying elements of the spectral distribution graph; and

calculating the one or more biomarkers based on one or more of the identified elements.

Clause 59b. The method of clause 59a, wherein the identified elements correspond to peaks in the spectral distribution graph.

Clause 60. The method of clause 48, wherein classifying the movement of the body part as normal movement or abnormal movement based on the one or more biomarkers comprises:

comparing the one or more biomarkers to one or more corresponding baseline biomarkers; and

determining normal movement of the body part in response to a comparison that satisfies a threshold criterion; and

determining abnormal movement of the body part in response to a comparison that does not satisfy the threshold criterion.

Clause 61. The method of clause 60, wherein the corresponding baseline biomarkers are derived from one or more representations of normal movement of a body part of the same type as the representation of movement of the body part of the patient.

Clause 62. The method of clause 61, wherein the one or more representations of normal movement are obtained across a patient population.

Clause 63. The method of clause 61, wherein the one or more representations of normal movement are obtained from the patient.

Clause 64. The method of clause 48, wherein:

the body part of the patient corresponds to a leg,

normal movement of the body part of a patient corresponds to normal walking, and

abnormal movement of the body part of a patient corresponds to one of limping, limping with pain, limping with limited range of motion.

Clause 65. The method of any one of clause 48-64, wherein the method is implemented by a computer.

Clause 66. A classification apparatus comprising:

a memory; and

a processor coupled to the memory and configured to:
obtain a representation of movement of a body part of a patient;
derive one or more metrics from the representation; and
classify the movement of the body part as normal movement or abnormal movement based on the one or more metrics.

Clause 67. The classification apparatus of clause 66, wherein the processor is further configured to implement the methods of any one of clauses 48-65.

Clause 67a. An implantable medical device for diagnosing a kinematic condition, the device comprising:

a sensor configured to acquire kinematic data indicative of motion activity of a body part of a patient;

a memory coupled to the sensor and configured to store a record of acquired kinematic data; and

a processor coupled to the memory and configured to applying a machine-learned classification model to the record to classify the motion activity of the body part as a type of movement.

Clause 68. A method comprising:

obtaining, from across a patient population, a plurality of raw kinematic data corresponding to movement of a body part;

transforming the plurality of raw kinematic data into a corresponding plurality of processed kinematic data; and

training a machine learning model on the plurality of processed transformed kinematic data to identify a plurality of elements within the kinematic data.

Clause 70. The method of clause 68, wherein:

the raw kinematic data comprises motion data from a single channel of a multi-channel inertial measurement unit; and

transforming the raw kinematic data comprises filtering the raw kinematic data.

Clause 71. The method of clause 68, wherein:

the raw kinematic data comprises individual motion data from a plurality of channels of a multi-channel inertial measurement unit; and

transforming the raw kinematic data comprises fusing the individual motion data from the plurality of channels into fused motion data.

Clause 72. The method of clause 71, wherein transforming the raw kinematic data further comprises one of:

filtering the fused motion data; or

filtering the individual motion data from the plurality of channels prior to combining the individual motion data.

Clause 73. The method of anyone of clauses 70, 71, and 72, wherein the multi-channel inertial measurement unit comprises a gyroscope oriented relative to the body part and configured to provide as raw kinematic data, a signal corresponding to angular velocity about one or more axes relative to the body part.

Clause 74. The method of anyone of clauses 70, 71, and 72, wherein the multi-channel inertial measurement unit comprises an accelerometer oriented relative to the body part and configured to provide as kinematic data, a signal corresponding to acceleration along one or more axes relative to the body part.

Clause 75. The method of any one of clause 68-74, wherein the method is implemented by a computer.

Clause 76. A training apparatus comprising:

a memory; and

a processor coupled to the memory and configured to:
obtain, from across a patient population, a plurality of raw kinematic data corresponding to movement of a body part;
transform the plurality of raw kinematic data into a corresponding plurality of processed kinematic data; and
train a machine learning model on the plurality of processed transformed kinematic data to identify a plurality of elements within the kinematic data.

Clause 77. The training apparatus of clause 76, wherein the processor is further configured to implement the methods of any one of clauses 69-75.

Clause 78. A method comprising:

obtaining, from across a patient population, a plurality of kinematic data corresponding to movement of a body part, each signal characterized by a plurality of elements corresponding to a point in a motion cycle; and

training a machine learning model on the plurality of kinematic data to quantify a kinematic variable or a kinematic parameter.

Clause 79. The method of clause 78, wherein the kinematic variable comprises one of: a time interval between pairs of points, ratios of time intervals between pairs of points, elevations of points relative to a baseline of a time-series waveform, and differences in elevations between a pair of point.

Clause 80. The method of clause 78, wherein the kinematic parameter comprises one of: cadence, stride length, walking speed, tibia range of motion, knee range of motion, step count and distance traveled.

Clause 81. The method of clauses 78-80 implemented by a computer.

Clause 82. A training apparatus comprising:

a memory; and

a processor coupled to the memory and configured to:
obtain, from across a patient population, a plurality of kinematic data corresponding to movement of a body part, each signal characterized by a plurality of elements corresponding to a point in a motion cycle; and
train a machine learning model on the plurality of kinematic data to quantify a kinematic variable or a kinematic parameter.

Clause 89. A method of assessing movement of a person having a sensor associated with a leg, the method comprising:

capturing data over time through the sensor that is representative of one or more of acceleration and rotation of a portion of the leg;

processing the data to identify the data as corresponding to a qualified gait of the person;

deriving one or more kinematic biomarkers from the data; and

evaluating conditions of the person based on the one or more kinematic biomarkers and corresponding baseline kinematic biomarkers.

Clause 89a. The method of clause 89, wherein the biomarkers comprise kinematic variables derived from visual representations of the data.

Clause 89b. The method of clause 89, wherein the biomarkers comprise kinematic parameters derived from measures of one or more of acceleration and rotation of the portion of the leg.

Clause 89c. The method of clause 89, wherein the baseline kinematic biomarkers represent normal gait and evaluating comprises applying a machine-learned algorithm trained to quantifying a difference between the derived biomarkers and the baseline biomarkers.

Clause 90. A method comprising:

obtaining a plurality of datasets for a corresponding plurality of patients, the plurality of datasets comprising kinematic data of motion activity of a body part that has undergone surgery;

obtaining a plurality of measures of a kinematic parameter based on the kinematic data as a function of time since the surgery; and

deriving a plurality of benchmark curves based on the plurality of measures as a function of time and percentile.

Clause 90a. The method of clause 90, wherein obtaining a plurality of measures of a kinematic parameter comprises:

representing each of the kinematic data in a visual form; and

applying a machine-learned algorithm to each of the visual forms, wherein the machine-learned algorithm is trained to output a quantification of the kinematic parameter.

Clause 91. A method comprising:

obtaining kinematic data from a plurality of intelligent implants across a patient population, each intelligent implant implanted in a patient, the kinematic data obtained from one or more sensors of the intelligent implant and indicative of patient activity;

monitor the kinematic data over time to identify a plurality of subsets of the patient population, where each patient in a subset of the patient population has similar kinematic data;

assigning a data sampling configuration to each identified subset of the patient population; and

providing a signal configured to set the data sampling configuration of an intelligent implant implanted in a patient based on the subset of the patient population within which the patient falls.

Clause 92. The method of clause 91, wherein each patient in a subset of the patient population has kinematic data indicative of activity at or above a threshold during a same first period of time and kinematic data indicative of inactivity at or below a threshold during a same second period of time.

Clause 93. The method of clause 92, wherein the data sampling configuration configures the intelligent implant to sample data from the one or more sensors during the first time period, in accordance with a sampling schedule.

Clause 94. The method of clause 92, wherein the data sampling configuration configures the intelligent implant to refrain from sampling data from the one or more sensors during the second time period.

Clause 95. The method of clause 91, wherein the one or more sensors of the intelligent implant are configured to trigger data sampling and recording upon occurrence of a threshold force, and obtaining kinematic data from a plurality of intelligent implants across a patient population comprises:

identifying one or more patients whose associated intelligent implant provides no kinematic data; and

adjusting a sensitivity of the sensor to require less force to trigger data sampling and recording.

Clause 96. The method of clause 91, wherein the one or more sensors of the intelligent implant are configured to trigger data sampling and recording upon occurrence of a threshold force, and obtaining kinematic data from a plurality of intelligent implants across a patient population comprises:

identifying one or more patients whose associated intelligent implant provides kinematic data indicative of persistent walking; and

adjusting a sensitivity of the sensor to require more force to trigger data sampling and recording.

Clause 97. A method comprising:

obtaining raw kinematic data corresponding to movement of a body part of a patient, wherein the raw kinematic data is obtained from a sensor implanted in or on the body part;

transforming the raw kinematic data to video animation data; and

displaying an animation corresponding to the movement of the body part based on the video animation data.

Clause 98. The method of clause 97, further comprising:

applying a machine-learned algorithm to the raw kinematic data, wherein the algorithm is trained to derive one or more gait parameters based on the raw kinematic data; and

displaying the one or more gait parameters.

Clause 99. The method of clause 97, further comprising:

applying a machine-learned algorithm to the raw kinematic data, wherein the algorithm is trained to derive a gait classification based on the raw kinematic data; and

displaying the gait classification.

Clause 100. A computer-implemented method for identifying an orthopedic condition of an individual, comprising:

obtaining kinematic data of an individual;

deriving one or more kinematic features from the kinematic data;

evaluating the one or more kinematic features using a machine-learning classification model to generate a determination of the orthopedic condition; and

providing the determination to the individual or a third party.

Clause 103. The computer-implemented method of clause 100, wherein the one or more kinematic features comprises a variable derived from a plurality of elements identified in a time-series waveform representation of the kinematic data.

Clause 104. The computer-implemented method of clause 100, wherein the one or more kinematic features comprise a variable derived from a plurality of elements identified a spectral distribution representation of the kinematic data.

Clause 105. The computer-implemented method of clause 100, wherein the determination has a quantification of the determined orthopedic condition

Clause 106. An apparatus for determining an orthopedic condition of a patient, comprising:

a processor; and

a memory storing computer executable instructions, which when executed by the processor cause the processor to perform operations comprising:
obtaining patient kinematic data of the patient;
deriving one or more patient kinematic features from the patient kinematic data; and
determining the orthopedic condition based on the one or more patient kinematic features using a machine-learning classification model trained on a training set of kinematic features of the same type as the patient kinematic features.

Clause 109. The apparatus of clause 106, wherein the one or more patient kinematic features comprises a variable derived from a plurality of elements identified in a time-series waveform representation of the kinematic data.

Clause 110. The apparatus of clause 106, wherein the one or more patient kinematic features comprise a variable derived from a plurality of elements identified a spectral distribution representation of the kinematic data.

Clause 111. The apparatus of clause 106, wherein the patient kinematic data is obtained from at least one sensor associated with a body part of the patient.

Clause 112. The apparatus of clause 111, wherein the at least one sensor is implanted in or adjacent the body part.

Clause 113. The apparatus of clause 111, wherein the at least one sensor is external the patient and position on or adjacent the body part.

Clause 114. A method comprising:

receiving kinematic data indicative of a movement of a body part of a patient;

deriving one or more kinematic features from the kinematic data; and

applying a machine-learning classification model determined based on supervised machine learning to classify the movement of the body part based on the one or more kinematic features.

Clause 115. A system comprising:

at least one sensor adapted to acquire kinematic data indicative of a movement of a body part of an ambulatory patient in a non-clinical setting; and

a processor comprising a machine-learning classification model, the processor adapted to:
derive one or more kinematic features from the acquired kinematic data; and
apply the machine-learning classification model to the one or more kinematic features to classify the movement of the body part;
calculate a quantification score of the movement of the body part based at least in part on the acquired movement data.

Clause 116. The system of clause 115, wherein the machine-learning classification model is trained at least in part on a training dataset across a patient population, the training data comprising:

kinematic features extracted from kinematic data acquired across a patient population using at least one sensor of the same type as the at least one sensor of the ambulatory patient; and

a label associated with the kinematic features.

Clause 117. The system of clause 115, wherein the label associated with the kinematic features is a supervised label assigned by an expert.

Clause 118. The system of clause 115, wherein the label associated with the kinematic features is an unsupervised label assigned by a clustering algorithm.

Clause 119. An apparatus for predicting an outcome of a patient, comprising:

a processor; and

a memory storing computer executable instructions, which when executed by the processor cause the processor to perform operations comprising:
obtaining patient kinematic data of the patient;
deriving one or more patient kinematic features from the patient kinematic data; and
determining the outcome based on the one or more patient kinematic features and at least one additional data element of the patient using an outcome model trained on a training set of kinematic features of the same type as the patient kinematic features and the at least one additional data element.

Clause 120. The apparatus of clause 119, wherein the one or more patient kinematic features comprise at least one of:

a time-series waveform representation of the patient kinematic data,

a time-series variable derived from the time-series waveform,

a spectral-distribution graph of the patient kinematic data,

a spectral-distribution variable derived from the spectral-distribution graph,

a kinematic parameter derived based on acceleration and angular velocity measurements included in the kinematic data.

Clause 121. The apparatus of clause 120, wherein the time-series variable comprises one of time intervals between elements of the time-series waveform, ratios based on one or more of the intervals, elevation (or offset) of a kinematic feature relative to a reference line, and elevation difference between different elements.

Clause 122. The apparatus of clause 120, wherein the spectral-distribution variable comprises a peak frequency in the spectral-distribution graph.

Clause 123. The apparatus of clause 120, wherein the kinematic parameter comprises one or more of cadence, stride length, walking speed, tibia range of motion, knee range of motion, step count and distance traveled.

Clause 124. The apparatus of clause 119, wherein the at least one additional data element comprises one or more of demographic data, medical data, device operation data; clinical outcome data; clinical movement data; and non-kinematic data.

Clause 125. The apparatus of clause 119, wherein the outcome model comprises a statistical model.

Clause 126. The apparatus of clause 119, wherein the outcome model comprises a machine-learned model.

Clause 127. The apparatus of clause 119, wherein the outcome model comprises a deep learning machine-learned model.

Clause 128. The apparatus of clause 119, wherein the outcome comprises one or more of a movement classification, a risk of infection, a recovery state, a recovery prediction, etc.

Clause 129. The apparatus of clause 128, wherein the outcome comprises a quantification of the one or more movement classification, risk of infection, recovery state, recovery prediction, etc.

Clause 130. A method of determining an orientation of a medical device placed relative to a body part, wherein the medical device has a device coordinate system, and the body part has an anatomical coordinate system, the method comprising:

calculating a transverse plane skew angle between corresponding transverse planes of the device coordinate system and the anatomical coordinate system;

responsive to a transverse plane skew angle that is less than a threshold value, determining that the device coordinate system is aligned with the anatomical coordinate system; and

responsive to a transverse plane skew angle that is above the threshold value, determining that the device coordinate system is not aligned with the anatomical coordinate system.

Clause 131. The method of clause 130, wherein the threshold value is in the range of 1 degree to 8 degrees.

Clause 132. The method of clause 130, wherein the threshold value is in the range of 1 degree to 4 degrees.

Clause 133. The method of clause 130, wherein the threshold value is 1 degree.

Clause 134. A device configured to be secured to a limb, such as a lower leg, of a subject, the device comprising a plurality of sensors located within a housing of the device, the plurality of sensors comprising a gyroscope and an accelerometer that detect acceleration, tilt, vibration, shock and/or rotation, where the gyroscope and accelerometer optionally capture data samples between 25 Hz and 1,600 Hz, e.g., between 50 Hz and 800 Hz.

Clause 135. The device of clause 134 wherein the plurality of sensors further comprises a magnetometer located within the device.

Clause 136. The device of clause 134 further comprising an electronic processor positioned within the device that is electrically coupled to the plurality of sensors.

Clause 137. The device of clause 134 further comprising a first memory coupled to an electronic processor and configured to receive data from the at least one sensor, and optionally comprising a second memory coupled to an electronic processor and configured to store firmware.

Clause 138. The device of clause 134 further comprising a telemetry circuit including an antenna to transmit data from the memory to a location outside of the device.

Clause 139. The device of clause 138 wherein the telemetry circuit is configured to communicate with a second device via a short-range network protocol, such as the medical implant communication service (MICS), the medical device radio communications service (MedRadio), or some other wireless communication protocol such as, e.g., Bluetooth.

Clause 140. The device of clause 134 wherein the housing is configured to comprise a shape that is complementary to a shape of the outer surface of a subject's body, e.g., the front surface of a lower leg so the device may rest against a tibia and maintain a constant orientation vis-h-vis the tibia, a surface of the upper arm so the device may rest adjacent to a humerus and maintain a constant orientation vis-h-vis the humerus, the front surface of an upper leg so the device may rest adjacent to a femur and maintain a constant orientation vis-h-vis the femur.

Clause 141. The device of clause 134 further comprising a fuse positioned between the power supply and at least one of the kinematic sensor, the memory and the telemetric circuit.

Clause 142. A device configured to be secured to a limb of a mammal, the device comprising a sensor selected from an accelerometer and a gyroscope, a memory configured to store data obtained from the sensor, a telemetry circuit configured to transmit data stored in the memory; and a battery configured to provide power to the sensor, memory and telemetry circuit, where the gyroscope and accelerometer optionally capture data samples between 25 Hz and 1,600 Hz, e.g., between 50 Hz and 800 Hz, and where the limb is optionally a front surface of a lower leg so the device may rest against a tibia and maintain a constant orientation vis-h-vis the tibia, or the limb is optionally a surface of the upper arm so the device may rest adjacent to a humerus and maintain a constant orientation vis-h-vis the humerus, or the limb is optionally a surface of an upper leg so the device may rest adjacent to a femur and maintain a constant orientation vis-h-vis the femur.

Clause 143. The device of clause 142 wherein the telemetry circuit is configured to communicate with a second device via a short-range network protocol, such as the medical implant communication service (MICS), the medical device radio communications service (MedRadio), or some other wireless communication protocol such as, e.g., Bluetooth.

Clause 144. A device for measuring kinematic movement, the device comprising:
a housing configured to be securely held to an outer surface of a limb, e.g., a lower leg, of an animal,
a plurality of electrical components contained within the housing, the plurality of electrical components comprising:
   a first sensor configured to sense movement of the limb, e.g., lower leg, and obtain a periodic measure of the movement of the limb and generate a first signal that reflects the periodic measure of the movement,
   a second sensor configured to sense movement of the limb, e.g., lower leg and obtain a continuous measure of the movement of the limb and generate a second signal that reflects the continuous measure of the movement;
a memory configured to store data corresponding to the second signal but not the first signal;
a telemetry circuit configured to transmit data corresponding to the second signal stored in the memory; and
a battery configured to provide power to the plurality of electrical components.

Clause 145. The device of clause 144 wherein the housing is attached to a strap that goes around the lower leg to secure the housing to the outer surface of the lower leg.

Clause 146. The device of clause 144 wherein the housing is attached to a strap that is configured to go around an upper leg to secure the housing to the outer surface of the upper leg, or wherein the housing is attached to a strap that is configured to go around an upper arm to secure the hosing to the outer surface of the upper arm.

Clause 147. The device of clause 144 wherein the housing comprises a region with a polymeric surface and the telemetry circuit comprises an antenna that is positioned under the polymeric surface of the housing, to allow transmission of the data corresponding to the second signal through the polymeric surface and to a location separate from the device.

Clause 148. The device of clause 144 wherein the telemetry circuit is configured to communicate with a second device via a short-range network protocol, such as the medical implant communication service (MICS), the medical device radio communications service (MedRadio), or some other wireless communication protocol such as Bluetooth.

Clause 149. A non-surgical method comprising:

obtaining data, the data comprising acceleration data from accelerometers positioned within the device of clauses 134-148, and/or rotation data from gyroscopes positioned within the device of clauses 134-148;

storing the data in a memory located in the device; and

transferring the data from said memory to a memory in a second device.

Clause 150. The method of clause 149 wherein the telemetry circuit transfers the accelerometer and gyroscope data to a second device via a short-range network protocol, such as the medical implant communication service (MICS), the medical device radio communications service (MedRadio), or some other wireless communication protocol such as Bluetooth.

Clause 151. A non-surgical method for detecting and/or recording an event in a subject with a device according to clauses 134 to 148 secured thereto, comprising the step of interrogating at a desired point in time the activity of one or more sensors within the device, and recording said activity.

Clause 152. The method according to clause 151 wherein the step of interrogating is performed by a health care provider.

Clause 153. The method according to clause 151 wherein said recording is provided to a health care provider.

Clause 154. A method for imaging a movement a limb comprising a joint replacement prosthesis, e.g., a knee of a leg, to which a device of any one of clauses 134-148 is secured, comprising the steps of:

detecting the location of one or more sensors in the device of clauses 134-148; and

visually displaying the location of said one or more sensors, such that an image of the joint replacement prosthesis is created; and optionally providing said image to a health care provider.

Clause 155. The method of clause 154 wherein the step of detecting occurs over time.

Clause 156. The method of clause 154 wherein said visual display shows changes in the positions of said sensors over time.

Clause 157. A system comprising
a first device according to any of clauses 134-148; and
a second device that is implanted within the subject, where the second device comprises a sensor selected from an accelerometer and a gyroscope, a memory configured to store data obtained from the kinematic sensor, a telemetry circuit configured to transmit data stored in the memory; and a battery configured to provide power to the sensor, memory and telemetry circuit.

Clause 158. The system of clause 157 wherein the first and second devices communicate with each other via a short-range network protocol, such as the medical implant communication service (MICS), the medical device radio communications service (MedRadio), or some other wireless communication protocol such as Bluetooth.

Clause 159. The system of clause 157 wherein first and second devices each communicate with a third device such as a base station, via a short-range network protocol, such as the medical implant communication service (MICS), the medical device radio communications service (MedRadio), or some other wireless communication protocol such as Bluetooth.

Clause 160. The system of clause 158 wherein the first and second devices communicate with each other via a 402 MHz to 405 MHz MICS band.

Clause 161. The system of clause 158 wherein an accelerometer and a gyroscope are each located within each of the first and second devices.

Clause 162. The system of clause 158 wherein the second device is a knee implant located within a leg of the subject and the first device is configured to be secured to the leg of the subject.

Clause 163. The system of clause 158 wherein the second device is a hip implant located within a hip of the subject and the first device is configured to be secured to the leg that attaches to the side of the hip of the subject that has the hip implant.

Clause 164. The system of clause 158 wherein the second device is a shoulder implant located within a shoulder of a subject and the first device is configured to be secured to the arm that attaches to shoulder of the subject that has the implant.

Clause 165. A computer-implemented method for generating a patient movement classification model, wherein the computer-implemented method comprises, as implemented by a computing system comprising one or more computer processors:
obtaining a plurality of records from across a patient population, wherein a record of the plurality of records comprises kinematic data representing motion of an implant implanted in a patient of the patient population, and wherein the implant comprises a plurality of sensors configured to detect motion of the implant;
for individual records of the plurality of records:
   identifying one or more elements represented by the kinematic data;
   determining one or more kinematic features based on the one or more elements; and
   labeling the one or more kinematic features with a movement type of a plurality of movement types to generate one or more labeled kinematic features, wherein each movement type of the plurality of movement types is associated with movement of a body part; and
training a machine learning model using the labeled kinematic features to classify motion of a particular implant as a particular movement type.

Clause 166. The computer-implemented method of clause 165, wherein identifying one or more elements represented by the kinematic data comprises:
representing the kinematic data as a time-series waveform, and
identifying a set of fiducial points in the time-series waveform, wherein the one or more elements correspond to the set of fiducial points.

Clause 167. The computer-implemented method of clause 166, wherein movement of the body part corresponds to a gait cycle, and wherein the one or more elements correspond to points in the gait cycle that correspond to one of a heel-strike, a loading response, a mid-stance, a terminal stance, a pre-swing, a toe-off, a mid-swing, and a terminal swing.

Clause 168. The computer-implemented method of any of clauses 165-167, wherein the body part is associated with a body joint comprising one of a hip joint, knee joint, ankle joint, shoulder joint, elbow joint, and wrist joint.

Clause 169. The computer-implemented method of any of clauses 165-167, wherein kinematic data for a particular patient is obtained from only a single implant implanted into a first bone of a plurality of bones of a particular body joint of the particular patient.

Clause 170. The computer-implemented method of any of clauses 165-167, wherein the implant comprises a tibial implant.

Clause 171. The computer-implemented method of any of clauses 165-167, further comprising:
representing each kinematic data included in the plurality of records as one of a time-series waveform or a spectral distribution graph; and
applying a clustering algorithm to a plurality of time-series waveforms or spectral distribution graphs to automatically separate the plurality of time-series waveforms or spectral distribution graphs into a plurality of clusters;
wherein labeling the one or more kinematic features with a movement type is based determining that the one or more kinematic features are associated with a particular cluster of the plurality of clusters.

Clause 172. The computer-implemented method of clause 165, wherein a first sensor of the plurality of sensors comprises a gyroscope oriented relative to the body part and configured to provide, as kinematic data, a signal representing angular velocity about a first axis relative to the body part.

Clause 173. The computer-implemented method of clause 165, wherein a first sensor of the plurality of sensors comprises an accelerometer oriented relative to the body part and configured to provide, as kinematic data, a signal representing acceleration along a first axis relative to the body part.

Clause 174. The computer-implemented method of any of clauses 172 or 173, wherein the first axis is one axis of a three-dimensional implant coordinate system comprising a second axis and a third axis, and wherein obtaining the plurality of records comprises:
obtaining from a second sensor of the plurality of sensors, as kinematic data, a signal representing one of: angular velocity about the second axis relative to the body part, or acceleration along the second axis relative to the body part; and
obtaining from a third sensor of the plurality of sensors, as kinematic data, a signal representing one of: angular velocity about the third axis relative to the body part, or acceleration along the third axis relative to the body part.

Clause 175. The computer-implemented method of clause 174, further comprising, prior to labeling the one or more kinematic features, combining two or more of the respective signals representing angular velocity or acceleration about the first axis, the second axis, and the third axis.

Clause 176. The computer-implemented method of clause 174, further comprising:
calculating a transverse plane skew angle between corresponding transverse planes of the implant coordinate system and an anatomical coordinate system associated with the body part;
responsive to a transverse plane skew angle that is less than a threshold value, determining that the implant coordinate system is aligned with the anatomical coordinate system; and
responsive to a transverse plane skew angle that is above the threshold value, determining that the implant coordinate system is not aligned with the anatomical coordinate system.

Clause 177. The computer-implemented method of any of clauses 165-167, wherein the plurality of records further comprises one or more of: patient demographic data, patient medical data, implant operation data, clinical outcome data, clinical movement data, non-kinematic data, unsupervised labels, or supervised labels.

Clause 178. The computer-implemented method of any of clauses 165-167, further comprising:
obtaining a plurality of datasets for a corresponding plurality of patients, the plurality of datasets comprising kinematic data of motion activity of a body part that has undergone surgery;
generating a plurality of measures of a kinematic parameter based on the kinematic data as a function of time since the surgery; and
generating a plurality of benchmark curves based on the plurality of measures as a function of time and percentile.

Clause 179. A system comprising:
an implant configured to be implanted into a patient, wherein the implant comprises a plurality of sensors configured to detect motion of the implant; and
one or more computer processors programmed by executable instructions to at least:
   receive a plurality of records from the implant, wherein a record of the plurality of records comprises kinematic data representing motion of the implant;
   determine one or more kinematic features based on the kinematic data;
   determine, based at least partly on the one or more kinematic features, a movement type of a plurality of movement types, wherein the movement type is associated with movement of a body part of the patient.

Clause 180. The system of clause 179, wherein a sensor of the plurality of sensors is configured to sample motion of the patient according to a plurality of sample rates, and wherein an assigned sample rate is changed from a first lower sample rate of the plurality of sample rates to a second higher sample rate of the plurality of sample rates in response to a movement detection event.

Clause 181. The system of clause 179, wherein a sensor of the plurality of sensors is configured to sample motion of the patient according to a plurality of sample rates, and wherein an assigned sample rate is changed from a first higher sample rate of the plurality of sample rates to a second lower sample rate of the plurality of sample rates based on a scheduled time.

Clause 182. The system of clause 179, where the one or more computer processors are further programmed by the executable instructions to:
determine a biomarker based on at least one of the kinematic data or the movement type;
compare the biomarker to a baseline biomarker; and
determine a patient recovery state based on a result of comparing the biomarker to the baseline biomarker.

Clause 183. The system of clause 179, wherein the biomarker comprises a kinematic feature derived from a time-series representation or a spectral distribution representation of the kinematic data, or a kinematic parameter derived based on acceleration and angular velocity measurements included in the kinematic data.

Clause 184. The system of clause 183, wherein the kinematic feature comprises one of: time intervals between elements, ratios based on one or more of the time intervals, offset of a kinematic feature relative to a reference line, and elevation difference between different elements.

Clause 185. The system of any of clauses 179-184, wherein the one or more computer processors are further programmed by the executable instructions to generate a user interface comprising:

a plurality of patient recovery trajectory curves representing respective benchmarks of recovery from a type of surgery as a function of time; and

a patient recovery trajectory curve representing recovery of the patient from the type of surgery as a function of time.

The devices, methods, systems etc. of the present disclosure have been described broadly and generically herein. Each of the narrower species and subgeneric groupings falling within the generic disclosure also form part of the present disclosure. This includes the generic description of the devices, methods, systems etc. of the present disclosure with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the present disclosure are described in terms of Markush groups, it is intended, and those skilled in the art will recognize, that the present disclosure embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. For example, the term “a sensor” refers to one or more sensors, and the term “a medical device comprising a sensor” is a reference to a medical device that includes at least one sensor. A plurality of sensors refers to more than one sensor. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the disclosure, invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the disclosure, invention or claims. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the present disclosure. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the disclosure pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

As mentioned above, in the following claims, the terms used should not be construed to limit the claims to the specific embodiments disclosed in the specification and the claims, but should be construed to include all possible embodiments along with the full scope of equivalents to which such claims are entitled. For example, described embodiments with one or more omitted components or steps can be additional embodiments contemplated and covered by this application.

1. A computer-implemented method for generating a patient movement classification model, wherein the computer-implemented method comprises, as implemented by a computing system comprising one or more computer processors:
obtaining a plurality of records from across a patient population, wherein a record of the plurality of records comprises kinematic data representing motion of an implant implanted in a patient of the patient population, and wherein the implant comprises a plurality of sensors configured to detect motion of the implant;
for individual records of the plurality of records:
identifying one or more elements represented by the kinematic data;
determining one or more kinematic features based on the one or more elements; and
labeling the one or more kinematic features with a movement type of a plurality of movement types to generate one or more labeled kinematic features, wherein each movement type of the plurality of movement types is associated with movement of a body part; and
training a machine learning model using the labeled kinematic features to classify motion of a particular implant as a particular movement type.

2. The computer-implemented method of claim 1, wherein identifying one or more elements represented by the kinematic data comprises:
representing the kinematic data as a time-series waveform, and
identifying a set of fiducial points in the time-series waveform, wherein the one or more elements correspond to the set of fiducial points.

3. (canceled)

4. The computer-implemented method of claim 1, wherein the body part is associated with a body joint comprising one of a hip joint, knee joint, ankle joint, shoulder joint, elbow joint, and wrist joint.

5. (canceled)

6. (canceled)

7. The computer-implemented method of claim 1, further comprising:
representing each kinematic data included in the plurality of records as one of a time-series waveform or a spectral distribution graph; and
applying a clustering algorithm to a plurality of time-series waveforms or spectral distribution graphs to automatically separate the plurality of time-series waveforms or spectral distribution graphs into a plurality of clusters;
wherein labeling the one or more kinematic features with a movement type is based determining that the one or more kinematic features are associated with a particular cluster of the plurality of clusters.

8. The computer-implemented method of claim 1, wherein a first sensor of the plurality of sensors comprises a gyroscope oriented relative to the body part and configured to provide, as kinematic data, a signal representing angular velocity about a first axis relative to the body part.

9. The computer-implemented method of claim 1, wherein a first sensor of the plurality of sensors comprises an accelerometer oriented relative to the body part and configured to provide, as kinematic data, a signal representing acceleration along a first axis relative to the body part.

10. The computer-implemented method of claim 8, wherein the first axis is one axis of a three-dimensional implant coordinate system comprising a second axis and a third axis, and wherein obtaining the plurality of records comprises:
obtaining from a second sensor of the plurality of sensors, as kinematic data, a signal representing one of: angular velocity about the second axis relative to the body part, or acceleration along the second axis relative to the body part; and
obtaining from a third sensor of the plurality of sensors, as kinematic data, a signal representing one of: angular velocity about the third axis relative to the body part, or acceleration along the third axis relative to the body part.

11. The computer-implemented method of claim 10, further comprising, prior to labeling the one or more kinematic features, combining two or more of the respective signals representing angular velocity or acceleration about the first axis, the second axis, and the third axis.

12. The computer-implemented method of claim 10, further comprising:
calculating a transverse plane skew angle between corresponding transverse planes of the implant coordinate system and an anatomical coordinate system associated with the body part;
responsive to a transverse plane skew angle that is less than a threshold value, determining that the implant coordinate system is aligned with the anatomical coordinate system; and
responsive to a transverse plane skew angle that is above the threshold value, determining that the implant coordinate system is not aligned with the anatomical coordinate system.

13. (canceled)

14. (canceled)

15. A system comprising:
an implant configured to be implanted into a patient, wherein the implant comprises a plurality of sensors configured to detect motion of the implant; and
one or more computer processors programmed by executable instructions to at least:
receive a plurality of records from the implant, wherein a record of the plurality of records comprises kinematic data representing motion of the implant;
determine one or more kinematic features based on the kinematic data;
determine, based at least partly on the one or more kinematic features, a movement type of a plurality of movement types, wherein the movement type is associated with movement of a body part of the patient.

16. The system of claim 15, wherein a sensor of the plurality of sensors is configured to sample motion of the patient according to a plurality of sample rates, and wherein an assigned sample rate is changed from a first lower sample rate of the plurality of sample rates to a second higher sample rate of the plurality of sample rates in response to a movement detection event.

17. The system of claim 15, wherein a sensor of the plurality of sensors is configured to sample motion of the patient according to a plurality of sample rates, and wherein an assigned sample rate is changed from a first higher sample rate of the plurality of sample rates to a second lower sample rate of the plurality of sample rates based on a scheduled time.

18. The system of claim 15, where the one or more computer processors are further programmed by the executable instructions to:
determine a biomarker based on at least one of the kinematic data or the movement type;
compare the biomarker to a baseline biomarker; and
determine a patient recovery state based on a result of comparing the biomarker to the baseline biomarker.

19. The system of claim 18, wherein the biomarker comprises a kinematic feature derived from a time-series representation or a spectral distribution representation of the kinematic data, or a kinematic parameter derived based on acceleration and angular velocity measurements included in the kinematic data.

20. The system of claim 19, wherein the kinematic feature comprises one of: time intervals between elements, ratios based on one or more of the time intervals, offset of a kinematic feature relative to a reference line, and elevation difference between different elements.

21. The system of claim 15, wherein the one or more computer processors are further programmed by the executable instructions to generate a user interface comprising:
a plurality of patient recovery trajectory curves representing respective benchmarks of recovery from a type of surgery as a function of time; and
a patient recovery trajectory curve representing recovery of the patient from the type of surgery as a function of time.

22. (canceled)

23. (canceled)

24. A device for measuring kinematic movement, the device comprising:
a housing configured to be securely held to an outer surface of a limb, e.g., a lower leg, of an animal,
a plurality of electrical components contained within the housing, the plurality of electrical components comprising:
a first sensor configured to sense movement of the limb and obtain a periodic measure of the movement of the limb and generate a first signal that reflects the periodic measure of the movement,
a second sensor configured to sense movement of the limb and obtain a continuous measure of the movement of the limb and generate a second signal that reflects the continuous measure of the movement;
a memory configured to store data corresponding to the second signal but not the first signal;
a telemetry circuit configured to transmit data corresponding to the second signal stored in the memory; and
a battery configured to provide power to the plurality of electrical components.

25. A non-surgical method comprising:
obtaining data, the data comprising acceleration data from an accelerometer positioned within the device of claim 24, and/or rotation data from a gyroscope positioned within the device of claim 24;
storing the data in a memory located in the device; and
transferring the data from said memory to a memory in a second device.

26. A non-surgical method for detecting and/or recording an event in a subject with a device according to claim 24 secured thereto, comprising the step of interrogating at a desired point in time the activity of one or more sensors within the device, and recording said activity.

27. A method for imaging a movement a limb comprising a joint replacement prosthesis, e.g., a leg, to which a device of claim 24 is secured, comprising the steps of:
detecting the location of one or more sensors in the device of claims 21, 22 or 23; and
visually displaying the location of said one or more sensors, such that an image of the joint replacement prosthesis is created.

28. (canceled)
